SEQUENCE LISTING (7 sequences)

SEQ ID NO: 1 - protein, 923 aa, Artificial Sequence
  2NLS-NgAgo WT NgAgo amended with N- and C-terminal sequences for optimal translation, nuclear localization, and antibody detection
MASSPKKKRKVMTVIDLDSTTTADELTSGHTYDISVTLTGVYDNTDEQHPRMSLAFEQDNGERRYITLWKNTTPKDVFTYDYATGSTYIFTNIDYEVKDGYENLTATYQTTVENATAQEVGTTDEDETFAGGEPLDHHLDDALNETPDDAETESDSGHVMTSFASRDQLPEWTLHTYTLTATDGAKTDTEYARRTLAYTVRQELYTDHDAAPVATDGLMLLTPEPLGETPLDLDCGVRVEADETRTLDYTTAKDRLLARELVEEGLKRSLWDDYLVRGIDEVLSKEPVLTCDEFDLHERYDLSVEVGHSGRAYLHINFRHRFVPKLTLADIDDDNIYPGLRVKTTYRPRRGHIVWGLRDECATDSLNTLGNQSVVAYHRNNQTPINTDLLDAIEAADRRVVETRRQGHGDDAVSFPQELLAVEPNTHQIKQFASDGFHQQARSKTRLSASRCSEKAQAFAERLDPVRLNGSTVEFSSEFFTGNNEQQLRLLYENGESVLTFRDGARGAHPDETFSKGIVNPPESFEVAVVLPEQQADTCKAQWDTMADLLNQAGAPPTRSETVQYDAFSSPESISLNVAGAIDPSEVDAAFVVLPPDQEGFADLASPTETYDELKKALANMGIYSQMAYFDRFRDAKIFYTRNVALGLLAAAGGVAFTTEHAMPGDADMFIGIDVSRSYPEDGASGQINIAATATAVYKDGTILGHSSTRPQLGEKLQSTDVRDIMKNAILGYQQVTGESPTHIVIHRDGFMNEDLDPATEFLNEQGVEYDIVEIRKQPQTRLLAVSDVQYDTPVKSIAAINQNEPRATVATFGAPEYLATRDGGGLPRPIQIERVAGETDIETLTRQVYLLSQSHIQVHNSTARLPITTAYADQASTHATKGYLVQTGAFESNVGFLKRPAATKKAGQAKKKKYPYDVPDYA

SEQ ID NO: 2 - protein, 1159 aa, Artificial Sequence
  2NLS-NgAgo-mNeonGreen WT NgAgo amended with N- and C-terminal sequences for optimal translation, nuclear localization, and antibody detection, and fluorescent reporter fusion
MASSPKKKRKVMTVIDLDSTTTADELTSGHTYDISVTLTGVYDNTDEQHPRMSLAFEQDNGERRYITLWKNTTPKDVFTYDYATGSTYIFTNIDYEVKDGYENLTATYQTTVENATAQEVGTTDEDETFAGGEPLDHHLDDALNETPDDAETESDSGHVMTSFASRDQLPEWTLHTYTLTATDGAKTDTEYARRTLAYTVRQELYTDHDAAPVATDGLMLLTPEPLGETPLDLDCGVRVEADETRTLDYTTAKDRLLARELVEEGLKRSLWDDYLVRGIDEVLSKEPVLTCDEFDLHERYDLSVEVGHSGRAYLHINFRHRFVPKLTLADIDDDNIYPGLRVKTTYRPRRGHIVWGLRDECATDSLNTLGNQSVVAYHRNNQTPINTDLLDAIEAADRRVVETRRQGHGDDAVSFPQELLAVEPNTHQIKQFASDGFHQQARSKTRLSASRCSEKAQAFAERLDPVRLNGSTVEFSSEFFTGNNEQQLRLLYENGESVLTFRDGARGAHPDETFSKGIVNPPESFEVAVVLPEQQADTCKAQWDTMADLLNQAGAPPTRSETVQYDAFSSPESISLNVAGAIDPSEVDAAFVVLPPDQEGFADLASPTETYDELKKALANMGIYSQMAYFDRFRDAKIFYTRNVALGLLAAAGGVAFTTEHAMPGDADMFIGIDVSRSYPEDGASGQINIAATATAVYKDGTILGHSSTRPQLGEKLQSTDVRDIMKNAILGYQQVTGESPTHIVIHRDGFMNEDLDPATEFLNEQGVEYDIVEIRKQPQTRLLAVSDVQYDTPVKSIAAINQNEPRATVATFGAPEYLATRDGGGLPRPIQIERVAGETDIETLTRQVYLLSQSHIQVHNSTARLPITTAYADQASTHATKGYLVQTGAFESNVGFLKRPAATKKAGQAKKKKYPYDVPDYAMVSKGEEDNMASLPATHELHIFGSINGVDFDMVGQGTGNPNDGYEELNLKSTKGDLQFSPWILVPHIGYGFHQYLPYPDGMSPFQAAMVDGSGYQVHRTMQFEDGASLTVNYRYTYEGSHIKGEAQVKGTGFPADGPVMTNSLTAADWCRSKKTYPNDKTIISTFKWSYTTGNGKRYRSTARTTYTFAKPMAANYLKNQPMYVFRKTELKHSKTELNFKEWQKAFTDVMGMDELYK

SEQ ID NO: 3 - DNA, 3311 bp, Artificial Sequence
  strong constitutive expression cassette for 2NLS-NgAgo
atggcgtcctccccaaagaagaagcgtaaggtcatgactgttatcgaccttgattctactacaaccgctgacgaacttacttccggacacacctacgacatttcggttactcttaccggcgtttacgacaatactgatgagcaacaccccaggatgtcccttgcattcgaacaagacaacggcgagagaaggtacatcactctgtggaaaaacactacacctaaggacgtgttcacctacgattacgcaaccgggagtacatacatctttacaaacatcgactacgaggtaaaggacgggtacgaaaacctaacagctacttaccagaccactgtcgagaatgctacagcccaagaggtgggcaccaccgacgaggatgaaacattcgccggaggtgaacctctggaccatcaccttgatgatgctttaaacgaaacccctgacgatgcagagactgagtccgactccggacacgtgatgacttcctttgcatctagggatcagctacctgagtggactcttcacacctacaccctgacagctactgacggagccaaaaccgatactgagtacgccaggcgtacccttgcttacacagtcagacaagaactatacactgaccatgatgccgctccagtcgctaccgatggactgatgcttcttacacctgaaccactgggcgaaacaccacttgaccttgattgcggcgtgagggtggaagccgacgaaactcgcacactggactacaccaccgctaaagatcggttactcgccagagagcttgtagaagagggacttaaacgtagtttatgggacgattaccttgttagaggtatcgacgaggtcctcagtaaggaacctgtccttacctgcgacgagtttgatcttcatgagaggtacgacctttctgtggaagtcggacattcggggagggcataccttcatattaacttccgtcatcgttttgtacctaaactaacactggctgacatcgacgatgacaacatttacccaggacttcgtgtcaaaacaacctaccggccccgtcgtggtcacattgtctggggacttcgggacgagtgcgcaacagactctcttaataccctcggaaaccaaagtgttgtggcttaccataggaacaaccaaacaccaattaacactgaccttctcgacgctatcgaagccgctgatcgccgggttgtggagacacgtagacaaggtcatggggacgacgctgtgtccttcccacaagagcttctggctgttgaacccaacacccatcagatcaagcaattcgcttccgatggcttccatcaacaagccaggtctaagacacgtctttcggcttctcggtgctccgagaaagcccaagcatttgctgaacgtcttgaccctgtccgtcttaacggctctactgtcgagtttagttccgagttcttcaccggaaacaatgaacagcaactgagacttctctacgaaaatggggaatcggtccttacatttcgtgatggagccaggggagcccatccagatgagacattctcgaaaggcattgtaaatccacccgaatcctttgaagtcgctgtcgtccttcctgaacaacaggctgatacctgcaaggctcagtgggacaccatggctgatctactcaaccaagcaggcgctcctcctacaaggagtgaaacagtccagtacgatgccttctccagtcccgagagtattagtcttaacgttgctggagccattgacccatccgaggtggatgccgctttcgtggtacttccaccagaccaagaaggattcgctgacctggcttccccaacagagacatacgacgaactgaaaaaggctcttgctaacatgggaatctacagtcaaatggcttacttcgaccgttttcgcgacgctaaaatcttctacacccgtaatgtcgcccttggcctgcttgcagccgctggaggtgtcgcatttacaacagaacatgctatgcctggagatgctgacatgtttatcgggatcgacgtttccaggtcttaccctgaagatggagccagcggacaaatcaacatcgcagctactgcaaccgctgtctacaaggacggaaccatccttggacacagttccactcgtccacaattaggagaaaaacttcaatccaccgatgtcagggatattatgaagaacgccatcctcggataccaacaagtgaccggagaatctcctacccacattgtgattcatcgtgacggcttcatgaacgaggacttagatcctgccacagagtttctaaacgaacaaggcgtcgagtacgatatcgttgaaattcgcaagcaacctcaaaccaggctattagccgtaagtgatgttcaatacgacacacctgtcaagtccattgctgctatcaaccaaaacgaaccacgcgctaccgtggccacctttggcgcccctgagtaccttgctacacgcgatggtggcggcttacctagacctattcaaatcgagcgcgtcgctggagaaacagatatcgaaactcttacaaggcaagtgtaccttctttctcagagtcacatccaggtccataactccaccgctcggctccctatcacaactgcctacgctgaccaggcttcgacccatgctacaaaaggatacttagtccaaaccggagcctttgaatccaacgtggggttcctgaagcgccctgctgccaccaaaaaggctggacaagccaaaaaaaagaagtacccatacgatgtaccagattacgcttaatctagaggtacctgatcatgagtaattagctcgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgctcgacgcggccgccatggcctctagtggatcacctagggtcgatcgacaagctcgagtttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatcccccgaatta

SEQ ID NO: 4 - DNA, 4019 bp, Artificial Sequence
  strong constitutive expression cassette for 2NLS-NgAgo-mNeonGreen
atggcgtcctccccaaagaagaagcgtaaggtcatgactgttatcgaccttgattctactacaaccgctgacgaacttacttccggacacacctacgacatttcggttactcttaccggcgtttacgacaatactgatgagcaacaccccaggatgtcccttgcattcgaacaagacaacggcgagagaaggtacatcactctgtggaaaaacactacacctaaggacgtgttcacctacgattacgcaaccgggagtacatacatctttacaaacatcgactacgaggtaaaggacgggtacgaaaacctaacagctacttaccagaccactgtcgagaatgctacagcccaagaggtgggcaccaccgacgaggatgaaacattcgccggaggtgaacctctggaccatcaccttgatgatgctttaaacgaaacccctgacgatgcagagactgagtccgactccggacacgtgatgacttcctttgcatctagggatcagctacctgagtggactcttcacacctacaccctgacagctactgacggagccaaaaccgatactgagtacgccaggcgtacccttgcttacacagtcagacaagaactatacactgaccatgatgccgctccagtcgctaccgatggactgatgcttcttacacctgaaccactgggcgaaacaccacttgaccttgattgcggcgtgagggtggaagccgacgaaactcgcacactggactacaccaccgctaaagatcggttactcgccagagagcttgtagaagagggacttaaacgtagtttatgggacgattaccttgttagaggtatcgacgaggtcctcagtaaggaacctgtccttacctgcgacgagtttgatcttcatgagaggtacgacctttctgtggaagtcggacattcggggagggcataccttcatattaacttccgtcatcgttttgtacctaaactaacactggctgacatcgacgatgacaacatttacccaggacttcgtgtcaaaacaacctaccggccccgtcgtggtcacattgtctggggacttcgggacgagtgcgcaacagactctcttaataccctcggaaaccaaagtgttgtggcttaccataggaacaaccaaacaccaattaacactgaccttctcgacgctatcgaagccgctgatcgccgggttgtggagacacgtagacaaggtcatggggacgacgctgtgtccttcccacaagagcttctggctgttgaacccaacacccatcagatcaagcaattcgcttccgatggcttccatcaacaagccaggtctaagacacgtctttcggcttctcggtgctccgagaaagcccaagcatttgctgaacgtcttgaccctgtccgtcttaacggctctactgtcgagtttagttccgagttcttcaccggaaacaatgaacagcaactgagacttctctacgaaaatggggaatcggtccttacatttcgtgatggagccaggggagcccatccagatgagacattctcgaaaggcattgtaaatccacccgaatcctttgaagtcgctgtcgtccttcctgaacaacaggctgatacctgcaaggctcagtgggacaccatggctgatctactcaaccaagcaggcgctcctcctacaaggagtgaaacagtccagtacgatgccttctccagtcccgagagtattagtcttaacgttgctggagccattgacccatccgaggtggatgccgctttcgtggtacttccaccagaccaagaaggattcgctgacctggcttccccaacagagacatacgacgaactgaaaaaggctcttgctaacatgggaatctacagtcaaatggcttacttcgaccgttttcgcgacgctaaaatcttctacacccgtaatgtcgcccttggcctgcttgcagccgctggaggtgtcgcatttacaacagaacatgctatgcctggagatgctgacatgtttatcgggatcgacgtttccaggtcttaccctgaagatggagccagcggacaaatcaacatcgcagctactgcaaccgctgtctacaaggacggaaccatccttggacacagttccactcgtccacaattaggagaaaaacttcaatccaccgatgtcagggatattatgaagaacgccatcctcggataccaacaagtgaccggagaatctcctacccacattgtgattcatcgtgacggcttcatgaacgaggacttagatcctgccacagagtttctaaacgaacaaggcgtcgagtacgatatcgttgaaattcgcaagcaacctcaaaccaggctattagccgtaagtgatgttcaatacgacacacctgtcaagtccattgctgctatcaaccaaaacgaaccacgcgctaccgtggccacctttggcgcccctgagtaccttgctacacgcgatggtggcggcttacctagacctattcaaatcgagcgcgtcgctggagaaacagatatcgaaactcttacaaggcaagtgtaccttctttctcagagtcacatccaggtccataactccaccgctcggctccctatcacaactgcctacgctgaccaggcttcgacccatgctacaaaaggatacttagtccaaaccggagcctttgaatccaacgtggggttcctgaagcgccctgctgccaccaaaaaggctggacaagccaaaaaaaagaagtacccatacgatgtaccagattacgctatggtgagtaaaggagaagaagataacatggcttcgcttccagccacacatgagcttcacatcttcggttccatcaacggcgttgacttcgatatggtcggacaaggcactgggaaccctaatgacggatacgaagagctgaacctcaagagcaccaaaggtgatcttcagttttctccatggattctggtgccacacattggctacggattccatcaataccttccataccctgacggaatgagtccattccaagcagccatggttgatggctccggataccaagtccacaggacaatgcagtttgaggacggtgcttcgctcaccgtcaactaccgttacacttacgaagggagccacatcaaaggagaagcccaagtgaaggggacaggctttcctgctgatggacctgtcatgaccaactccttaactgccgctgattggtgccggtccaagaaaacctaccctaacgacaagaccatcattagtaccttcaaatggtcttacaccacaggcaatggcaagagatatcgctctacagccaggactacctacacattcgctaaaccaatggccgctaactaccttaagaaccaacccatgtacgtgttccgtaagactgagttgaaacattccaagaccgaacttaacttcaaggagtggcagaaggcatttaccgacgtaatgggcatggatgaactatacaaataatctagaggtacctgatcatgagtaattagctcgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgctcgacgcggccgccatggcctctagtggatcacctagggtcgatcgacaagctcgagtttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatcccccgaatta

SEQ ID NO: 5 - protein, 887 aa, Natronobacterium gregoryi
MTVIDLDSTTTADELTSGHTYDISVTLTGVYDNTDEQHPRMSLAFEQDNGERRYITLWKNTTPKDVFTYDYATGSTYIFTNIDYEVKDGYENLTATYQTTVENATAQEVGTTDEDETFAGGEPLDHHLDDALNETPDDAETESDSGHVMTSFASRDQLPEWTLHTYTLTATDGAKTDTEYARRTLAYTVRQELYTDHDAAPVATDGLMLLTPEPLGETPLDLDCGVRVEADETRTLDYTTAKDRLLARELVEEGLKRSLWDDYLVRGIDEVLSKEPVLTCDEFDLHERYDLSVEVGHSGRAYLHINFRHRFVPKLTLADIDDDNIYPGLRVKTTYRPRRGHIVWGLRDECATDSLNTLGNQSVVAYHRNNQTPINTDLLDAIEAADRRVVETRRQGHGDDAVSFPQELLAVEPNTHQIKQFASDGFHQQARSKTRLSASRCSEKAQAFAERLDPVRLNGSTVEFSSEFFTGNNEQQLRLLYENGESVLTFRDGARGAHPDETFSKGIVNPPESFEVAVVLPEQQADTCKAQWDTMADLLNQAGAPPTRSETVQYDAFSSPESISLNVAGAIDPSEVDAAFVVLPPDQEGFADLASPTETYDELKKALANMGIYSQMAYFDRFRDAKIFYTRNVALGLLAAAGGVAFTTEHAMPGDADMFIGIDVSRSYPEDGASGQINIAATATAVYKDGTILGHSSTRPQLGEKLQSTDVRDIMKNAILGYQQVTGESPTHIVIHRDGFMNEDLDPATEFLNEQGVEYDIVEIRKQPQTRLLAVSDVQYDTPVKSIAAINQNEPRATVATFGAPEYLATRDGGGLPRPIQIERVAGETDIETLTRQVYLLSQSHIQVHNSTARLPITTAYADQASTHATKGYLVQTGAFESNVGFL

SEQ ID NO: 6 - protein, 887 aa, Natronobacterium gregoryi; identical to SEQ ID NO: 5

SEQ ID NO: 7 - DNA, 2664 bp, Natronobacterium gregoryi
atgacagtgattgacctcgattcgaccaccaccgcagacgaactgacatcgggacacacgtacgacatctcagtcacgctcaccggtgtctacgataacaccgacgagcagcatcctcgcatgtctctcgcattcgagcaggacaacggcgagcggcgttacattaccctgtggaagaacacgacacccaaggatgtctttacatacgactacgccacgggctcgacgtacatcttcactaacatcgactacgaagtgaaggacggctacgagaatctgactgcaacataccagacgaccgtcgagaacgctaccgctcaggaagtcgggacgactgacgaggacgaaacgttcgcgggcggcgagccgctcgaccatcacttggacgacgcgctcaatgagacgccagacgacgcggagacagagagcgactcaggccatgtgatgacctcgttcgcctcccgcgaccaactccctgagtggacgctgcatacgtatacgctaacagccacagacggcgcaaagacggacacggagtacgcgcgacgaaccctcgcatacacggtacggcaggaactctataccgaccatgatgcggctccggttgcaactgacgggctaatgcttctcacgccagagccgctcggcgagaccccgcttgacctcgattgcggtgtccgggtcgaggcggacgagactcggacactcgattacaccacggccaaagaccggttactcgcccgcgaactcgtcgaagaggggctcaaacgctccctctgggatgactacctcgttcgcggcatcgatgaagtcctctcaaaggagcctgtgctgacttgcgatgagttcgacctacatgagcggtatgacctctctgtcgaagtcggtcacagtgggcgggcgtaccttcacatcaacttccgccaccggttcgtaccgaagctgacgctcgcagacatcgatgatgacaacatctatcctgggctccgggtgaagacgacgtatcgcccccggcgaggacatatcgtctggggtctgcgggacgagtgcgccaccgactcgctcaacacgctgggaaaccagtccgtcgttgcataccaccgcaacaatcagacacctattaacactgacctcctcgacgctatcgaggccgctgaccggcgagtcgtcgaaacccgacgtcaagggcacggcgatgatgctgtctcattcccccaagaactgcttgcggtcgaaccgaatacgcaccaaattaagcagttcgcctccgacggattccaccaacaggcccgctcaaagacgcgtctctcggcctcccgctgcagcgagaaagcgcaagcgttcgccgagcggcttgacccggtgcgtctcaatgggtccacggtagagttctcctcggagtttttcaccgggaacaacgagcagcaactgcgcctcctctacgagaacggtgagtcggttctgacgttccgcgacggggcgcgtggtgcgcaccccgacgagacattctcgaaaggtatcgtcaatccaccagagtcgttcgaggtggccgtagtactgcccgagcagcaggcagatacctgcaaagcgcagtgggacacgatggctgacctcctcaaccaagctggcgcgccaccgacacggagcgagaccgtccaatatgatgcgttctcctcgccagagagcatcagcctcaatgtggctggagccatcgaccctagcgaggtagacgcggcattcgtcgtactgccgccggaccaagaaggattcgcagacctcgccagtccgacagagacgtacgacgagctgaagaaggcgcttgccaacatgggcatttacagccagatggcgtacttcgaccggttccgcgacgcgaaaatattctatactcgtaacgtggcactcgggctgctggcagccgctggcggcgtcgcattcacaaccgaacatgcgatgcctggggacgcagatatgttcattgggattgatgtctctcggagctaccccgaggacggtgccagcggccagataaacattgccgcgacggcgaccgccgtctacaaggatggaactatcctcggccactcgtccacccgaccgcagctcggggagaaactacagtcgacggatgttcgtgacattatgaagaatgccatcctcggctaccagcaggtgaccggtgagtcgccgacccatatcgtcatccaccgtgatggcttcatgaacgaagacctcgaccccgccacggaattcctcaacgaacaaggcgtcgagtacgacatcgtcgaaatccgcaagcagccccagacacgcctgctggcagtctccgatgtgcagtacgatacgcctgtgaagagcatcgccgctatcaaccagaacgagccacgggcaacggtcgccaccttcggcgcacccgaatacttagcgacacgcgatggaggcggccttccccgcccaatccaaattgaacgagtcgccggcgaaaccgacatcgagacgctcactcgccaagtctatctgctctcccagtcgcatatccaggtccataactcgactgcgcgcctacccatcaccaccgcatacgccgaccaggcaagtactcacgcgaccaagggttacctcgtccagaccggagcgttcgagtctaatgtcggattcctctaa

(19) United States
(12) Patent Application Publication
(10) Pub. No.: US 2017/0367280 A1
(43) Pub. Date: Dec. 28, 2017

(54) USE OF ARGONAUTE ENDONUCLEASES FOR EUKARYOTIC GENOME ENGINEERING

(71) Applicant: KWS SAAT SE, Einbeck (DE)

(72) Inventor: Aaron Hummel, St. Louis, MO (US)

(73) Assignee: KWS SAAT SE, Einbeck (DE)

(21) Appl. No.: 15/605,014

(22) Filed: May 25, 2017

Related U.S. Application Data
(60) Provisional application No. 62/342,548, filed on May 27, 2016.

(51) Int. Cl. A01H 1/06; C12N 15/10; C12N 15/82

(52) U.S. Cl. CPC A01H 1/06; C12N 15/829; C12N 15/8289; C12N 15/8286; C12N 15/8282; C12N 15/102; C12N 15/8274; C12N 15/8271; C12N 15/8241; C12N 15/8201; C12N 15/8281

(57) ABSTRACT

The present invention relates to the use of Argonaute systems in plants for genome engineering, and compositions used in such methods.

### CROSS-REFERENCE TO RELATED APPLICATIONS

This application claims priority to U.S. Provisional Application Ser. No. 62/342,548, filed on May 27, 2016, which is herein incorporated by reference in its entirety.

### BACKGROUND OF THE INVENTION

### 1. Technical Field

This invention relates to materials and methods for gene editing in eukaryotic cells, and particularly to methods for gene editing, that include for example and not limitation, using nucleic acid guided Argonaute systems.

### 2. Background and Related Art

The ability to precisely modify genetic material in eukaryotic cells enables a wide range of high value applications in medical, pharmaceutical, agricultural, basic research and other fields. Fundamentally, genome engineering provides this capability by introducing predefined genetic variation at specific locations in eukaryotic genomes, such as deleting, inserting, mutating, or substituting specific nucleic acid sequences. These alterations can be gene or location specific. However, a significant barrier to routine introduction of targeted genetic variation in eukaryotic cells is the absence of mutations, insertions, or rearrangements without a precursory break in the genome to stimulate changes. Targeted double-stranded breaks (DSBs) caused by expression of site-specific nucleases (SSNs) in plants, for example, can increase the frequency of homologous recombination (HR) at least two to three orders of magnitude (Puchta et al., Proc Natl Acad Sci USA 93:5055-5060, 1996). Thus, state of the art achievements in efficient gene editing for targeted mutagenesis, editing or insertions, are dependent on the ability to introduce genomic single- or double-strand breaks at specific locations in eukaryotic genomes. Efficient programmable endonuclease systems or SSNs are thereby fundamental for robust gene editing. Examples of SSNs that have been used for gene editing include homing endonucleases (also known as meganucleases), zinc-finger nucleases (ZFNs), transcription activator-like effector nucleases (TALENs), and clustered, regularly interspersed short palindromic repeat (CRISPR)/CRISPR-associated (CAS) nucleases. Among these systems, CRISPR/Cas is unique for its guide RNA component that enables target reprogramming that can be implemented more rapidly than the protein reengineering required to use the other systems.

The requirement for targeted introduction of chromosomal DSBs for efficient production of genetic variation renders SSNs essential in gene editing. Like CRISPR/Cas nucleases, Argonaute endonucleases (“Argonautes”) are involved in defense against foreign nucleic acids by using nucleic acid guides to specify a target sequence, which is then cleaved by the Argonaute protein component. Specifically, an Argonaute can bind and cleave a target nucleic acid by forming a complex with a designed or synthetic nucleic acid-targeting nucleic acid, where cleavage of the target nucleic acid can introduce double-stranded breaks in the target nucleic acid. Also like the Cas9 system, the Argonautes nucleic acid guides provide a facile method for programming endonuclease sequence specificity. However, short ssRNA molecules are used as guides by many eukaryotic Argonautes without any secondary structure recognition constraints, such as those present in the Cas9-short guide RNA (sgRNA) interaction. The abundance of ssRNA in most eukaryotic cells therefore makes specific targeting of RNA-guided eukaryotic Argonautes a potential challenge. In contrast, some prokaryotic Argonautes are guided by short 5′-phosphorylated ssDNA molecules (Swarts, D. C. et al. DNA-guided DNA interference by a prokaryotic Argonaute. Nature 507, 258-261, 2014; Swarts, D. C. et al. Argonaute of the archaeon Pyrococcus furiosus is a DNA-guided nuclease that targets cognate DNA. Nucleic Acids Res. 43, 5120-5129 2015), and therefore inherently have lower potential for misguiding by host cell-derived nucleic acids due to the scarcity of short ssDNA molecules present in eukaryotic cells. Thus, DNA-guided Argonaute endonucleases have potential for application in eukaryotic genome editing.

One such system was recently shown to be suitable for gene editing in human cells (Gao, F., Shen, X. Z., Jiang, F., Wu, Y., Han, C. (2016) DNA-guided genome editing using the Natronobacterium gregoryi Argonaute. Nat Biotech. advance online publication doi: 10.1038/nbt.3547). Use of the Natronobacterium gregoryi Argonaute (NgAgo) system in plants has not been previously demonstrated. Thus, this invention is based in part on the surprising discovery that NgAgo is active as an endonuclease at temperatures suitable for growth and culture of plants and plant cells and the further surprising discovery that the endonuclease can be used for gene editing in plant cells.

### SUMMARY OF THE INVENTION

As specified in the Background Section, there is a great need in the art to identify technologies for genome engineering, particularly in plants, and use this understanding to develop novel methods and compositions for such engineering. The present invention satisfies this and other needs. Embodiments of the present invention relate generally to methods and compositions for genome engineering and more specifically to use of the Argonaute system, including for example and not limitation the Argonaute protein system from Natronobacterium gregoryi to perform genome engineering in plants.

This invention is based in part on the discovery that nucleic acid-guided endonucleases of the Argonaute family can be used for plant genome engineering. Argonaute endonuclease systems share the advantage of CRISPR/Cas systems because they can be programmed for target specificity with a simple single-stranded nucleic acid. Thus, Argonaute endonuclease systems can be used without limitation to make targeted modifications in heritable material of eukaryotic cells including targeted insertions and deletions, targeted sequence replacements, targeted small- and large-scale genomic rearrangements including inversions or chromosome rearrangements, targeted edits of endogenous sequence, and targeted integration of foreign sequence. These modifications can be made independently or as simultaneous or sequential multiplex modifications within the cell. Thus, many valuable traits can be introduced into plants with an Argonaute endonuclease system.

The invention also provides a method for modifying genetic material present in a plant cell. The method can include delivering into the cell a nucleic acid-targeting nucleic acid that is targeted to a sequence of the cell's genetic material and an Argonaute endonuclease into a plant cell. The nucleic acid-targeting nucleic acid can then direct the Argonaute endonuclease to create breaks in the cell's genetic material at or near the target site specified by the nucleic acid-targeting nucleic acid. Repair of the breaks through the non-homologous end joining (NHEJ) or homologous recombination (HR) mediated pathways can result in targeted modifications in the genetic material of the plant cell. The nucleic acid-targeting nucleic acid and/or the Argonaute endonuclease can be delivered together or separately into plant cells via any suitable method including, for example and not limitation, by bacterial DNA-transfer such as Agrobacterium transformation, by microparticle bombardment, by polyethylene glycol (PEG) transformation, by electroporation, or by another suitable method, including mechanical introduction methods. Alternatively, an expression cassette for the Argonaute endonuclease can be stably integrated into the plant genome for heritable expression in the plant cell and its derivatives.

In addition to the advantages of a guide-DNA molecule, delivery of the NgAgo endonuclease is facilitated by its small size. The wildtype (WT) protein (GenBank Accession Number AFZ73749) is 887 amino acids, or roughly 2/3 the size of Streptococcus pyogenes Cas9. This simplifies cloning and vector assembly, can increase expression levels of the nuclease in cells, and reduces the challenge in expressing the protein from highly size-sensitive platforms such as viruses, including either DNA or RNA viruses.

The use of NgAgo for plant genome engineering is described herein. As demonstrated, and as a general process, transient test systems such as protoplasts can be used to analyze, validate, and optimize nuclease activity at episomal and endogenous or transgenic chromosomal targets. Modifications can also be made in regenerative or reproductive tissues, enabling production of gene edited plants and plant lines for basic research and agricultural applications.

Like other nucleic acid guided endonucleases, NgAgo SSNs usually require a minimum of two components for targeted mutagenesis in plant cells: a 5′-phosphorylated single-stranded guide-DNA and the NgAgo endonuclease protein. For targeted edits, insertions, or sequence replacements, a DNA template encoding the desired sequence changes can also be provided to the plant cell to introduce changes either via the NHEJ or HR repair pathways. Successful editing events are most commonly detected by phenotypic changes (such as by knockout or introduction of a gene that results in a visible phenotype), by PCR-based methods (such as by enrichment PCR, PCR-digest, or T7EI or Surveyor endonuclease assays), or by targeted Next Generation Sequencing (NGS; also known as deep sequencing).

One advantage of the NgAgo system over CRISPR/Cas is in the use of DNA as the guide nucleic acid instead of RNA. The lower cost of DNA synthesis, its higher inherent stability and reduced tendency to form secondary structures, and the many chemical modifications than can be added to DNA oligos provides a variety of advantages compared to use of a RNA or a guide RNA. Many modifications of synthesized DNA oligonucleotides are commercially available and can be useful for stabilizing the oligonucleotide in a host cell to prolong its availability for use by the Argonaute endonuclease in gene editing. Another advantage of the NgAgo system is that it is functional at temperatures suitable for growth and culture of plants and plant cells, such as for example and not limitation, about 20° C. to about 35° C., preferably about 23° C. to about 32° C., and most preferably about 25° C. to about 28° C.

In one aspect, the invention provides a method of modifying chromosomal or extrachromosomal genetic material in a eukaryotic cell, comprising:
a. introducing into the cell a nucleic acid-targeting nucleic acid that is directed against a target sequence within the cell chromosomal or extrachromosomal genetic material; and
b. introducing into the cell an Argonaute endonuclease that produces a single- or double-strand break at or near the target site of the nucleic acid-targeting nucleic acid.

In one embodiment of the methods of the invention, the nucleic acid-targeting nucleic acid is a 5′-phosphorylated, single-stranded DNA. In one embodiment of the methods of the invention, the nucleic acid-targeting nucleic acid has the length selected from the group consisting of 20, 21, 22, 23, 24, 25, 26, 27, 28, 29, and 30 nucleotides. The cell chromosomal or extrachromosomal genetic material includes, for example and not limitation, nuclear and organelle (e.g., mitochondrial) genetic material.

In one embodiment of the methods of the invention, the nucleic acid-targeting nucleic acid is comprised of conventional deoxyribonucleic acid nucleotides and standard phosphate backbone linkages. In one embodiment of the methods of the invention, the nucleic acid-targeting nucleic acid comprises unconventional and/or modified nucleotides and/or comprises unconventional and/or modified backbone chemistries. Non-limiting examples of modifications which can be used in nucleic acid-targeting nucleic acids in the methods of the invention include locked nucleic acid (LNA) bases, internucleotide phosphorothioate bonds in the backbone, 2′-O-Methyl RNA bases, unlocked nucleic acid (UNA) bases, inverted dT at the 3′ end, 5-Methyl dC bases, 5-hydroxybutynl-2′-deoxyuridine bases, 5-Nitroindole bases, deoxyInosine bases, 8-aza-7-deazaguanosine bases, Inverted Dideoxy-T at the 5′ end, Inverted dT at the 3′ end, Dideoxycytidine at the 3′ end, bases that increase specificity of homology-pairing with a target nucleic acid, bases that decrease specificity of homology-pairing with a target nucleic acid, bases that modulate the propensity for secondary structure formation by the nucleic acid-targeting nucleic acid, bases to prevent unwanted ligation of the guide-DNA into the genome, bases to prevent unwanted incorporation of the guide-DNA into the genome due to extension by DNA polymerases, and any combinations thereof.

In one embodiment of the methods of the invention, the Argonaute endonuclease is the Natronobacterium gregoryi Argonaute endonuclease (NgAgo) or a mutant or a derivative thereof. In one specific embodiment, the NgAgo is modified to express nickase activity or to have DNA targeting activity without any nickase or nuclease activity. In one specific embodiment, at least one additional protein domain with enzymatic activity is fused to the N- or C-terminus, or both, of the NgAgo endonuclease. Non-limiting examples of such additional protein domains include an exonuclease, a helicase, a domain involved in repair of DNA DSBs, a transcriptional (co-)activator, a transcriptional (co-)repressor, a methylase, a demethylase, and any combinations thereof.

In one embodiment of the methods of the invention, the amino acid sequence of Argonaute endonuclease has at least 70% similarity to SEQ ID NO: 5 (the sequence at NCBI Accession AFZ73749) or SEQ ID NO: 6.

In one embodiment of the methods of the invention, the Argonaute endonuclease is expressed or delivered as a heterologous polypeptide comprising translational fusion with one or more additional elements. Non-limiting examples of such additional elements localization signals, epitope tags, fluorescent reporters, mNeonGreen, GFP, enzymes involved in DNA break repair, and other functional domains.

In one embodiment of the methods of the invention, the Argonaute endonuclease is delivered as a DNA expression cassette configured for expression of the Argonaute endonuclease protein. In one specific embodiment, the DNA expression cassette is transiently delivered to the cell via an introduced nucleic acid. In another specific embodiment, the DNA expression cassette is stably incorporated into the genomic sequence of the cell or an ancestral cell, thereby providing heritable expression of the Argonaute endonuclease.

In one embodiment of the methods of the invention, the Argonaute endonuclease is delivered as an mRNA. In one embodiment of the methods of the invention, the Argonaute endonuclease is delivered as a protein. In one embodiment of the methods of the invention, the method comprises delivering a preassembled complex comprising the Argonaute endonuclease protein loaded with the nucleic acid-targeting nucleic acid prior to introduction into the cell.

In one embodiment of the methods of the invention, the eukaryotic cell is a plant cell. In one specific embodiment, the Argonaute endonuclease and/or the nucleic acid-targeting guide nucleic acid is delivered to the plant cell by a method selected from the group consisting of bacteria-mediated DNA transfer, microparticle bombardment into plant cells, polyethylene glycol (PEG) mediated transformation of plant cells, electroporation of plant cells, pollen-tube mediated introduction into zygotes, and delivery mediated by one or more cell-penetrating peptides (CPPs). In one specific embodiment, the Argonaute endonuclease and/or the nucleic acid-targeting guide nucleic acid is delivered to the plant cell by Agrobacterium-mediated transformation. In one specific embodiment, the plant cell is derived from a species selected from the group consisting of Hordeum vulgare, Hordeum bulbusom, Sorghum bicolor, Saccharum officinarium, Zea mays, Setaria italica, Oryza minuta, Oriza sativa, Oryza australiensis, Oryza alta, Triticum aestivum, Triticum durum, Secale cereale, Triticale, Malus domestica, Brachypodium distachyon, Hordeum marinum, Aegilops tauschii, Daucus glochidiatus, Beta vulgaris, Daucus pusillus, Daucus muricatus, Daucus carota, Eucalyptus grandis, Nicotiana sylvestris, Nicotiana tomentosiformis, Nicotiana tabacum, Nicotiana benthamiana, Solanum lycopersicum, Solanum tuberosum, Coffea canephora, Vitis vinifera, Erythrante guttata, Genlisea aurea, Cucumis sativus, Morus notabilis, Arabidopsis arenosa, Arabidopsis lyrata, Arabidopsis thaliana, Crucihimalaya himalaica, Crucihimalaya wallichii, Cardamine flexuosa, Lepidium virginicum, Capsella bursa pastoris, Olmarabidopsis pumila, Arabis hirsute, Brassica napus, Brassica oleracea, Brassica rapa, Raphanus sativus, Brassica juncacea, Brassica nigra, Eruca vesicaria subsp. sativa, Citrus sinensis, Jatropha curcas, Populus trichocarpa, Medicago truncatula, Cicer yamashitae, Cicer bijugum, Cicer arietinum, Cicer reticulatum, Cicer judaicum, Cajanus cajanifolius, Cajanus scarabaeoides, Phaseolus vulgaris, Glycine max, Gossypium sp., Astragalus sinicus, Lotus japonicas, Torenia fournieri, Allium cepa, Allium fistulosum, Allium sativum, Helianthus annuus, Helianthus tuberosus and Allium tuberosum, and any variety or subspecies belonging to one of the aforementioned plants. In one specific embodiment, the target sequence is selected from the group consisting of an acetolactate synthase (ALS) gene, an acetohydroxyacid synthase (AHAS) gene, an enolpyruvylshikimate phosphate synthase gene (EPSPS) gene, male fertility genes, male sterility genes (e.g., MS45, MS26, or MSCA1), female fertility genes, female sterility genes, male restorer genes, female restorer genes, genes associated with the traits of sterility, genes associated with the traits of fertility, genes associated with herbicide resistance, genes associated with herbicide tolerance, genes associated with fungal resistance, genes associated with viral resistance, genes associated with insect resistance, genes associated with drought tolerance, genes associated with chilling tolerance, genes associated with cold tolerance, genes associated with nitrogen use efficiency, genes associated with phosphorus use efficiency, genes associated with water use efficiency and genes associated with crop or biomass yield, and any mutants of such genes. In some embodiments, chromosomal or extrachromosomal genetic material of plant cells includes, for example and not limitation, nuclear genetic material, genetic material contained in a protoplast, and plastidic genetic material (e.g., chloroplast genetic material).

In one embodiment of the methods of the invention, the Argonaute endonuclease is modified so as to be active at a different temperature than its optimal temperature prior to modification. In one specific embodiment, the modified Argonaute endonuclease is active at temperatures suitable for growth and culture of plants and plant cells. In one specific embodiment, the modified Argonaute endonuclease is active at a temperature from about 20° C. to about 35° C. In one specific embodiment, the modified Argonaute endonuclease is active at a temperature from about 23° C. to about 32° C.

In one embodiment of the methods of the invention, the modification of chromosomal or extrachromosomal genetic material comprises enriching and excising target nucleic acids.

In conjunction with the above methods, the invention also provides plant cells modified by any of these methods and cells, whole plants, or progeny thereof derived from such modified cell.

In another aspect, the invention provides a kit comprising the Argonaute endonuclease as described in any of the foregoing methods, and at least one nucleic acid-targeting nucleic acid as described in any of the foregoing methods.

In a further aspect, the invention provides a composition comprising the Argonaute endonuclease as described in any of the foregoing methods, and at least one nucleic acid-targeting nucleic acid as described in any of the foregoing methods.

In another aspect, the invention provides a host cell comprising the Argonaute endonuclease as described in any of the foregoing methods, and at least one nucleic acid-targeting nucleic acid as described in any of the foregoing methods.

In yet another aspect, the invention provides a vector comprising a nucleic acid encoding the Argonaute endonuclease as described in any of the foregoing methods and at least one nucleic acid-targeting nucleic acid as described in any of the foregoing methods.

In a further aspect, the invention provides a method for treating a disease and/or condition and/or preventing insect infection/infestation in a plant comprising modifying chromosomal or extrachromosomal genetic material of said plant by use of any of the foregoing methods.

Non-limiting examples of the diseases and/or conditions treatable by the invented methods include Anthracnose Stalk Rot, Aspergillus Ear Rot, Common Corn Ear Rots, Corn Ear Rots (Uncommon), Common Rust of Corn, Diplodia Ear Rot, Diplodia Leaf Streak, Diplodia Stalk Rot, Downy Mildew, Eyespot, Fusarium Ear Rot, Fusarium Stalk Rot, Gibberella Ear Rot, Gibberella Stalk Rot, Goss's Wilt and Leaf Blight, Gray Leaf Spot, Head Smut, Northern Corn Leaf Blight, Physoderma Brown Spot, Pythium, Southern Leaf Blight, Southern Rust, and Stewart's Bacterial Wilt and Blight, and combinations thereof.

Non-limiting examples of the insects causing, directly or indirectly, diseases and/or conditions treatable by the invented methods include Armyworm, Asiatic Garden Beetle, Black Cutworm, Brown Marmorated Stink Bug, Brown Stink Bug, Common Stalk Borer, Corn Billbugs, Corn Earworm, Corn Leaf Aphid, Corn Rootworm, Corn Rootworm Silk Feeding, European Corn Borer, Fall Armyworm, Grape Colaspis, Hop Vine Borer, Japanese Beetle, Scouting for Fall Armyworm, Seedcorn Beetle, Seedcorn Maggot, Southern Corn Leaf Beetle, Southwestern Corn Borer, Spider Mite, Sugarcane Beetle, Western Bean Cutworm, White Grub, and Wireworms, and combinations thereof. The invented methods are also suitable for preventing infections and/or infestations of a plant by any such insect(s).

In another aspect, the invention provides a method for affecting at least one trait in a plant selected from the group consisting of sterility, fertility, herbicide resistance, herbicide tolerance, fungal resistance, viral resistance, insect resistance, drought tolerance, chilling tolerance, or cold tolerance, nitrogen use efficiency, phosphorus use efficiency, water use efficiency and crop or biomass yield, said method comprising modifying chromosomal or extrachromosomal genetic material of said plant by use of any of the foregoing methods.

These and other objects, features and advantages of the present invention will become more apparent upon reading the following specification in conjunction with the accompanying description and claims.

### DETAILED DESCRIPTION OF THE INVENTION

To facilitate an understanding of the principles and features of the various embodiments of the invention, various illustrative embodiments are explained below. Although exemplary embodiments of the invention are explained in detail, it is to be understood that other embodiments are contemplated. Accordingly, it is not intended that the invention is limited in its scope to the details of construction and arrangement of components set forth in the following description or examples. The invention is capable of other embodiments and of being practiced or carried out in various ways. Also, in describing the exemplary embodiments, specific terminology will be resorted to for the sake of clarity.

It must also be noted that, as used in the specification and the appended claims, the singular forms “a,” “an” and “the” include plural references unless the context clearly dictates otherwise. For example, reference to a component is intended also to include composition of a plurality of components. References to a composition containing “a” constituent is intended to include other constituents in addition to the one named. In other words, the terms “a,” “an,” and “the” do not denote a limitation of quantity, but rather denote the presence of “at least one” of the referenced item.

Also, in describing the exemplary embodiments, terminology will be resorted to for the sake of clarity. It is intended that each term contemplates its broadest meaning as understood by those skilled in the art and includes all technical equivalents which operate in a similar manner to accomplish a similar purpose.

Ranges may be expressed herein as from “about” or “approximately” or “substantially” one particular value and/or to “about” or “approximately” or “substantially” another particular value. When such a range is expressed, other exemplary embodiments include from the one particular value and/or to the other particular value. Further, the term “about” means within an acceptable error range for the particular value as determined by one of ordinary skill in the art, which will depend in part on how the value is measured or determined, i.e., the limitations of the measurement system. For example, “about” can mean within an acceptable standard deviation, per the practice in the art. Alternatively, “about” can mean a range of up to ±20%, preferably up to ±10%, more preferably up to ±5%, and more preferably still up to ±1% of a given value. Alternatively, particularly with respect to biological systems or processes, the term can mean within an order of magnitude, preferably within 2-fold, of a value. Where particular values are described in the application and claims, unless otherwise stated, the term “about” is implicit and in this context means within an acceptable error range for the particular value.

Similarly, as used herein, “substantially free” of something, or “substantially pure”, and like characterizations, can include both being “at least substantially free” of something, or “at least substantially pure”, and being “completely free” of something, or “completely pure”.

By “comprising” or “containing” or “including” is meant that at least the named compound, element, particle, or method step is present in the composition or article or method, but does not exclude the presence of other compounds, materials, particles, method steps, even if the other such compounds, material, particles, method steps have the same function as what is named.

Throughout this description, various components may be identified having specific values or parameters, however, these items are provided as exemplary embodiments. Indeed, the exemplary embodiments do not limit the various aspects and concepts of the present invention as many comparable parameters, sizes, ranges, and/or values may be implemented. The terms “first,” “second,” and the like, “primary,” “secondary,” and the like, do not denote any order, quantity, or importance, but rather are used to distinguish one element from another.

It is noted that terms like “specifically,” “preferably,” “typically,” “generally,” and “often” are not utilized herein to limit the scope of the claimed invention or to imply that certain features are critical, essential, or even important to the structure or function of the claimed invention. Rather, these terms are merely intended to highlight alternative or additional features that may or may not be utilized in a particular embodiment of the present invention. It is also noted that terms like “substantially” and “about” are utilized herein to represent the inherent degree of uncertainty that may be attributed to any quantitative comparison, value, measurement, or other representation.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “50 mm” is intended to mean “about 50 mm.”

It is also to be understood that the mention of one or more method steps does not preclude the presence of additional method steps or intervening method steps between those steps expressly identified. Similarly, it is also to be understood that the mention of one or more components in a composition does not preclude the presence of additional components than those expressly identified.

The materials described hereinafter as making up the various elements of the present invention are intended to be illustrative and not restrictive. Many suitable materials that would perform the same or a similar function as the materials described herein are intended to be embraced within the scope of the invention. Such other materials not described herein can include, but are not limited to, materials that are developed after the time of the development of the invention, for example.

In accordance with the present invention there may be employed conventional molecular biology, microbiology, and recombinant DNA techniques within the skill of the art. Such techniques are explained fully in the literature. See, e.g., Sambrook, Fritsch & Maniatis, Molecular Cloning: A Laboratory Manual, Second Edition (1989) Cold Spring Harbor Laboratory Press, Cold Spring Harbor, N.Y. (herein “Sambrook et al., 1989”); DNA Cloning: A Practical Approach, Volumes I and II (D. N. Glover ed. 1985); Oligonucleotide Synthesis (M. J. Gait ed. 1984); Nucleic Acid Hybridization (B. D. Hames & S. J. Higgins eds. (1985); Transcription and Translation (B. D. Hames & S. J. Higgins, eds. (1984); Animal Cell Culture (R. I. Freshney, ed. (1986); Immobilized Cells and Enzymes (IRL Press, (1986); B. Perbal, A Practical Guide To Molecular Cloning (1984); F. M. Ausubel et al. (eds.), Current Protocols in Molecular Biology, John Wiley & Sons, Inc. (1994); among others.

### Definitions

As used herein, “nucleic acid” means a polynucleotide and includes a single or a double-stranded polymer of deoxyribonucleotide or ribonucleotide bases. Nucleic acids may also include fragments and modified nucleotides. Thus, the terms “polynucleotide”, “nucleic acid sequence”, “nucleotide sequence” and “nucleic acid fragment” are used interchangeably to denote a polymer of RNA and/or DNA that is single- or double-stranded, optionally containing synthetic, non-natural, or altered nucleotide bases. Nucleotides (usually found in their 5′-monophosphate form) are referred to by their single letter designation as follows: “A” for adenosine or deoxyadenosine (for RNA or DNA, respectively), “C” for cytosine or deoxycytosine, “G” for guanosine or deoxyguanosine, “U” for uridine, “T” for deoxythymidine, “R” for purines (A or G), “Y” for pyrimidines (C or T), “K” for G or T, “H” for A or C or T, “I” for inosine, and “N” for any nucleotide. A nucleic acid can comprise nucleotides. A nucleic acid can be exogenous or endogenous to a cell. A nucleic acid can exist in a cell-free environment. A nucleic acid can be a gene or fragment thereof. A nucleic acid can be DNA. A nucleic acid can be RNA. A nucleic acid can comprise one or more analogs (e.g., altered backbone, sugar, or nucleobase). Some non-limiting examples of analogs include: 5-bromouracil, peptide nucleic acid, xeno nucleic acid, morpholinos, locked nucleic acids, glycol nucleic acids, threose nucleic acids, dideoxynucleotides, cordycepin, 7-deaza-GTP, florophores (e.g., rhodamine or flurescein linked to the sugar), thiol containing nucleotides, biotin linked nucleotides, fluorescent base analogs, CpG islands, methyl-7-guanosine, methylated nucleotides, inosine, thiouridine, pseudourdine, dihydrouridine, queuosine, and wyosine.

As used herein, the terms “Argonaute” or “Argonaute endonuclease” can be used interchangeably. An Argonaute can refer to any modified (e.g., shortened, mutated, lengthened) polypeptide sequence or homologue of the Argonaute, including variant, modified, fusion (as defined herein), and/or enzymatically inactive forms of the Argonaute. An Argonaute can be codon optimized. An Argonaute can be a codon-optimized homologue of an Argonaute. An Argonaute can be enzymatically inactive, partially active, constitutively active, fully active, inducibly active, active at different temperatures, and/or more active (e.g., more than the wild type homologue of the protein or polypeptide). In some instances, the Argonaute (e.g., variant, mutated, and/or enzymatically inactive Argonaute) can target a target nucleic acid. The Argonaute (e.g., variant, mutated, and/or enzymatically inactive) can target double-stranded or single-stranded DNA or RNA. The Argonaute can associate with a short targeting or guide nucleic acid that provides specificity for a target nucleic acid to be cleaved by the protein's endonuclease activity. The Argonaute can be provided separately or in a complex wherein it is pre-associated with the targeting or guide nucleic acid. In some instances, the Argonaute can be a fusion as described herein.

As used herein, the terms “Natronobacterium gregoryi Argonaute” or “NgAgo” are used interchangeably to refer to a DNA-guided endonuclease isolated from N. gregoryi that is suitable for genome editing. NgAgo binds 5′ phosphorylated single-stranded guide DNA of at least 10 to about 30 nucleotides in length, preferably at least 20 to about 30 nucleotides, and most preferably about 24 nucleotides, and efficiently creates site-specific DNA double-strand breaks when loaded with the guide-DNA. The NgAgo-guide-DNA system does not require a protospacer-adjacent motif (PAM), as does Cas9, and has a low tolerance to guide-target nucleic acid mismatches and high efficiency in editing (G+C)-rich genomic targets. The NgAgo is active at temperatures that are suitable for genome engineering in plants. An exemplary amino acid sequence of NgAgo is provided in GenBank Accession No. AFZ73749. The NgAgo is functional at a temperature range that is also suitable for growth and culture of plants and plant cells, such as for example and not limitation, about 20° C. to about 35° C., preferably about 23° C. to about 32° C., and most preferably about 25° C. to about 28° C. The NgAgo may be used in place of Argonaute in any of the embodiments described herein.

As used herein, “nucleic acid-targeting nucleic acid” or “nucleic acid-targeting guide nucleic acid” or “guide-DNA” or “guide-RNA” are used interchangeably and can refer to a nucleic acid that can bind an Argonaute protein of the disclosure and hybridize with a target nucleic acid. A nucleic acid-targeting nucleic acid can be RNA or DNA, including, without limitation, single-stranded RNA, double-stranded RNA, single-stranded DNA, and double-stranded DNA. The nucleic acid-targeting nucleic acid can bind to a target nucleic acid site-specifically. A portion of the nucleic acid-targeting nucleic acid can be complementary to a portion of a target nucleic acid. A nucleic acid-targeting nucleic acid can comprise a segment that can be referred to as a “nucleic acid-targeting segment.” A nucleic acid-targeting nucleic acid can comprise a segment that can be referred to as a “protein-binding segment.” The nucleic acid-targeting segment and the protein-binding segment can be the same segment of the nucleic acid-targeting nucleic acid. The nucleic acid-targeting nucleic acid may contain modified nucleotides, a modified backbone, or both. The nucleic acid-targeting nucleic acid may comprise a peptide nucleic acid (PNA).

As used herein, “donor polynucleotide” can refer to a nucleic acid that can be integrated into a site during genome engineering, target nucleic acid engineering, or during any other method of the disclosure.

As used herein, “fusion” can refer to a protein and/or nucleic acid comprising one or more non-native sequences (e.g., moieties). A fusion can be at the N-terminal or C-terminal end of the modified protein, or both. A fusion can be a transcriptional and/or translational fusion. A fusion can comprise one or more of the same non-native sequences. A fusion can comprise one or more of different non-native sequences. A fusion can be a chimera. A fusion can comprise a nucleic acid affinity tag. A fusion can comprise a barcode. A fusion can comprise a peptide affinity tag. A fusion can provide for subcellular localization of the Argonaute (e.g., a nuclear localization signal (NLS) for targeting to the nucleus, a mitochondrial localization signal for targeting to the mitochondria, a chloroplast localization signal for targeting to a chloroplast, an endoplasmic reticulum (ER) retention signal, and the like). A fusion can provide a non-native sequence (e.g., affinity tag) that can be used to track or purify. A fusion can be a small molecule such as biotin or a dye such as alexa fluor dyes, Cyanine3 dye, Cyanine5 dye. The fusion can provide for increased or decreased stability. In some embodiments, a fusion can comprise a detectable label, including a moiety that can provide a detectable signal. Suitable detectable labels and/or moieties that can provide a detectable signal can include, but are not limited to, an enzyme, a radioisotope, a member of a specific binding pair; a fluorophore; a fluorescent reporter or fluorescent protein; a quantum dot; and the like. A fusion can comprise a member of a FRET pair, or a fluorophore/quantum dot donor/acceptor pair. A fusion can comprise an enzyme. Suitable enzymes can include, but are not limited to, horse radish peroxidase, luciferase, beta-galactosidase, and the like. A fusion can comprise a fluorescent protein. Suitable fluorescent proteins can include, but are not limited to, a green fluorescent protein (GFP), (e.g., a GFP from Aequoria victoria, fluorescent proteins from Anguilla japonica, or a mutant or derivative thereof), a red fluorescent protein, a yellow fluorescent protein, a yellow-green fluorescent protein (e.g., mNeonGreen derived from a tetrameric fluorescent protein from the cephalochordate Branchiostoma lanceolatum) any of a variety of fluorescent and colored proteins. A fusion can comprise a nanoparticle. Suitable nanoparticles can include fluorescent or luminescent nanoparticles, and magnetic nanoparticles. Any optical or magnetic property or characteristic of the nanoparticle(s) can be detected.

A fusion can comprise a helicase, a nuclease (e.g., FokI), an endonuclease, an exonuclease (e.g., a 5′ exonuclease and/or 3′ exonuclease), a ligase, a nickase, a nuclease-helicase (e.g., Cas3), a DNA methyltransferase (e.g., Dam), or DNA demethylase, a histone methyltransferase, a histone demethylase, an acetylase (including for example and not limitation, a histone acetylase), a deacetylase (including for example and not limitation, a histone deacetylase), a phosphatase, a kinase, a transcription (co-) activator, a transcription (co-) factor, an RNA polymerase subunit, a transcription repressor, a DNA binding protein, a DNA structuring protein, a long noncoding RNA, a DNA repair protein (e.g., a protein involved in repair of either single and/or double-stranded breaks, e.g., proteins involved in base excision repair, nucleotide excision repair, mismatch repair, NHEJ, HR, microhomology-mediated end joining (MMEJ), and/or alternative non-homologous end-joining (ANHEJ), such as for example and not limitation, HR regulators and HR complex assembly signals), a marker protein, a reporter protein, a fluorescent protein, a ligand binding protein (e.g., mCherry or a heavy metal binding protein), a signal peptide (e.g., Tat-signal sequence), a targeting protein or peptide, a subcellular localization sequence (e.g., nuclear localization sequence, a chloroplast localization sequence), and/or an antibody epitope, or any combination thereof.

As used herein, “genome engineering” can refer to a process of modifying a target nucleic acid. Genome engineering can refer to the integration of non-native nucleic acid into native nucleic acid. Genome engineering can refer to the targeting of an Argonaute and a nucleic acid-targeting nucleic acid to a target nucleic acid, without an integration or a deletion of the target nucleic acid. Genome engineering can refer to the cleavage of a target nucleic acid, and the rejoining of the target nucleic acid without an integration of an exogenous sequence in the target nucleic acid, or a deletion in the target nucleic acid. The native nucleic acid can comprise a gene. The non-native nucleic acid can comprise a donor polynucleotide. In the methods of the disclosure, Argonautes, or complexes thereof, can introduce double-stranded breaks in a nucleic acid, (e.g. genomic DNA). The double-stranded break can stimulate a cell's endogenous DNA-repair pathways (e.g., homologous recombination (HR) and/or non-homologous end joining (NHEJ), or A-NHEJ (alternative non-homologous end-joining)). Mutations, deletions, alterations, and integrations of foreign, exogenous, and/or alternative nucleic acid can be introduced into the site of the double-stranded DNA break.

As used herein, the term “isolated” can refer to a nucleic acid or polypeptide that, by the hand of a human, exists apart from its native environment and is therefore not a product of nature. Isolated can mean substantially pure. An isolated nucleic acid or polypeptide can exist in a purified form and/or can exist in a non-native environment such as, for example, in a transgenic cell.

As used herein, “non-native” can refer to a nucleic acid or polypeptide sequence that is not found in a native nucleic acid or protein. Non-native can refer to affinity tags. Non-native can refer to fusions. Non-native can refer to a naturally occurring nucleic acid or polypeptide sequence that comprises mutations, insertions and/or deletions. A non-native sequence may exhibit and/or encode for an activity (e.g., enzymatic activity, methyltransferase activity, acetyltransferase activity, kinase activity, ubiquitinating activity, etc.) that can also be exhibited by the nucleic acid and/or polypeptide sequence to which the non-native sequence is fused. A non-native nucleic acid or polypeptide sequence may be linked to a naturally-occurring nucleic acid or polypeptide sequence (or a variant thereof) by genetic engineering to generate a chimeric nucleic acid and/or polypeptide sequence encoding a chimeric nucleic acid and/or polypeptide. A non-native sequence can refer to a 3′ hybridizing extension sequence.

As used herein, “nucleotide” can generally refer to a base-sugar-phosphate combination. A nucleotide can comprise a synthetic nucleotide. A nucleotide can comprise a synthetic nucleotide analog. Nucleotides can be monomeric units of a nucleic acid sequence (e.g. deoxyribonucleic acid (DNA) and ribonucleic acid (RNA)). The term nucleotide can include ribonucleoside triphosphates adenosine triphosphate (ATP), uridine triphosphate (UTP), cytosine triphosphate (CTP), guanosine triphosphate (GTP) and deoxyribonucleoside triphosphates such as dATP, dCTP, dITP, dUTP, dGTP, dTTP, or derivatives thereof. Such derivatives can include, for example and not limitation, [αS]dATP, 7-deaza-dGTP and 7-deaza-dATP, and nucleotide derivatives that confer nuclease resistance on the nucleic acid molecule containing them. The term nucleotide as used herein can refer to dideoxyribonucleoside triphosphates (ddNTPs) and their derivatives. Illustrative examples of dideoxyribonucleoside triphosphates can include, but are not limited to, ddATP, ddCTP, ddGTP, ddITP, and ddTTP. A nucleotide may be unlabeled or detectably labeled by well-known techniques. Labeling can also be carried out with quantum dots. Detectable labels can include, for example, radioactive isotopes, fluorescent labels, chemiluminescent labels, bioluminescent labels and enzyme labels. Fluorescent labels of nucleotides may include but are not limited to fluorescein, 5-carboxyfluorescein (FAM), 2′7′-dimethoxy-4′5-dichloro-6-carboxyfluorescein (JOE), rhodamine, 6-carboxyrhodamine (R6G), N,N,N′,N′-tetramethyl-6-carboxyrhodamine (TAMRA), 6-carboxy-X-rhodamine (ROX), 4-(4′dimethylaminophenylazo) benzoic acid (DABCYL), Cascade Blue, Oregon Green, Tex. Red, Cyanine and 5-(2′-aminoethyl)aminonaphthalene-1-sulfonic acid (EDANS).

As used herein, “recombinant” can refer to sequence that originates from a source foreign to the particular host (e.g., cell) or, if from the same source, is modified from its original form. A recombinant nucleic acid in a cell can include a nucleic acid that is endogenous to the particular cell but has been modified through, for example, the use of site-directed mutagenesis. The term can include non-naturally occurring multiple copies of a naturally occurring DNA sequence. Thus, the term can refer to a nucleic acid that is foreign or heterologous to the cell, or homologous to the cell but in a position or form within the cell in which the nucleic acid is not ordinarily found. Similarly, when used in the context of a polypeptide or amino acid sequence, an exogenous polypeptide or amino acid sequence can be a polypeptide or amino acid sequence that originates from a source foreign to the particular cell or, if from the same source, is modified from its original form.

As used herein, the term “specific” can refer to interaction of two molecules where one of the molecules through, for example chemical or physical means, specifically binds to the second molecule. Exemplary specific binding interactions can refer to antigen-antibody binding, avidin-biotin binding, carbohydrates and lectins, complementary nucleic acid sequences (e.g., hybridizing), complementary peptide sequences including those formed by recombinant methods, effector and receptor molecules, enzyme cofactors and enzymes, enzyme inhibitors and enzymes, and the like. “Non-specific” can refer to an interaction between two molecules that is not specific.

As used herein, “target nucleic acid” or “target site” can generally refer to a target nucleic acid to be targeted in the methods of the disclosure. A target nucleic acid can refer to a nuclear chromosomal/genomic sequence or an extrachromosomal sequence, (e.g., an episomal sequence, a minicircle sequence, a mitochondrial sequence, a chloroplast sequence, a protoplast sequence, a plastid sequence, etc.). A target nucleic acid can be DNA. A target nucleic acid can be single-stranded DNA. A target nucleic acid can be double-stranded DNA. A target nucleic acid can be single-stranded or double-stranded RNA. A target nucleic acid can herein be used interchangeably with “target nucleotide sequence” and/or “target polynucleotide”.

As used herein, “sequence identity” or “identity” in the context of nucleic acid or polypeptide sequences refers to the nucleic acid bases or amino acid residues in two sequences that are the same when aligned for maximum correspondence over a specified comparison window.

As used herein, the term “percentage of sequence identity” refers to the value determined by comparing two optimally aligned sequences over a comparison window, wherein the portion of the polynucleotide or polypeptide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the results by 100 to yield the percentage of sequence identity. Useful examples of percent sequence identities include, but are not limited to, 50%, 55%, 60%, 65%, 70%, 75%, 80%, 85%, 90% or 95%, or any integer percentage from 50% to 100%.

As used herein, the term “plant” refers to whole plants, plant organs, plant tissues, seeds, plant cells, seeds and progeny of the same. Plant cells include, without limitation, cells from seeds, suspension cultures, embryos, zygotes, meristematic regions, callus tissue, leaves, roots, shoots, gametophytes, protoplasts, plastids, sporophytes, pollen and microspores. Plant parts include differentiated and undifferentiated tissues including, but not limited to roots, stems, shoots, leaves, pollen, seeds, flowers, parts consumable by humans and/or other mammals (e.g., rice grains, corn cobs, tubers), tumor tissue and various forms of cells and culture (e.g., single cells, protoplasts, plastids, embryos, zygotes, and callus tissue). The plant tissue may be in plant or in a plant organ, tissue or cell culture. The term “plant organ” refers to plant tissue or a group of tissues that constitute a morphologically and functionally distinct part of a plant. The term “genome” refers to the entire complement of genetic material (genes and non-coding sequences) that is present in each cell of an organism, or virus or organelle; and/or a complete set of chromosomes inherited as a (haploid) unit from one parent. “Progeny” comprises any subsequent generation of a plant.

As used herein, the term “transgenic plant” includes, for example, a plant which comprises within its genome a heterologous polynucleotide introduced by a transformation step. The heterologous polynucleotide can be stably integrated within the genome such that the polynucleotide is passed on to successive generations. The heterologous polynucleotide may be integrated into the genome alone or as part of a recombinant DNA construct. A transgenic plant can also comprise more than one heterologous polynucleotide within its genome. Each heterologous polynucleotide may confer a different trait to the transgenic plant. A heterologous polynucleotide can include a sequence that originates from a foreign species, or, if from the same species, can be substantially modified from its native form. Transgenic can include any cell, cell line, callus, tissue, plant part or plant, the genotype of which has been altered by the presence of heterologous nucleic acid including those transgenics initially so altered as well as those created by sexual crosses or asexual propagation from the initial transgenic. The alterations of the genome (chromosomal or extra-chromosomal) by conventional plant breeding methods, by the genome editing procedure described herein that does not result in an insertion of a foreign polynucleotide, or by naturally occurring events such as random cross-fertilization, non-recombinant viral infection, non-recombinant bacterial transformation, non-recombinant transposition, or spontaneous mutation are not intended to be regarded as transgenic.

In certain embodiments of the disclosure, a fertile plant is a plant that produces viable male and female gametes and is self-fertile. Such a self-fertile plant can produce a progeny plant without the contribution from any other plant of a gamete and the genetic material contained therein. Other embodiments of the disclosure can involve the use of a plant that is not self-fertile because the plant does not produce male gametes, or female gametes, or both, that are viable or otherwise capable of fertilization. As used herein, a “male sterile plant” is a plant that does not produce male gametes that are viable or otherwise capable of fertilization. As used herein, a “female sterile plant” is a plant that does not produce female gametes that are viable or otherwise capable of fertilization. It is recognized that male-sterile and female-sterile plants can be female-fertile and male-fertile, respectively. It is further recognized that a male fertile (but female sterile) plant can produce viable progeny when crossed with a female fertile plant and that a female fertile (but male sterile) plant can produce viable progeny when crossed with a male fertile plant.

As used herein, the terms “plasmid”, “vector” and “cassette” refer to an extra-chromosomal element often carrying genes that are not part of the central metabolism of the cell, and usually in the form of double-stranded DNA. Such elements may be autonomously replicating sequences, genome integrating sequences, phage, or nucleotide sequences, in linear or circular form, of a single- or double-stranded DNA or RNA, derived from any source, in which a number of nucleotide sequences have been joined or recombined into a unique construction which is capable of introducing a polynucleotide of interest into a cell. “Transformation cassette” refers to a specific vector containing a gene and having elements in addition to the gene that facilitates transformation of a particular host cell. “Expression cassette” refers to a specific vector containing a gene and having elements in addition to the gene that allow for expression of that gene in a host.

The terms “recombinant DNA molecule”, “recombinant construct”, “expression construct”, “construct”, “construct”, and “recombinant DNA construct” are used interchangeably herein. A recombinant construct comprises an artificial combination of nucleic acid fragments, e.g., regulatory and coding sequences that are not all found together in nature. For example, a construct may comprise regulatory sequences and coding sequences that are derived from different sources, or regulatory sequences and coding sequences derived from the same source, but arranged in a manner different than that found in nature. Such a construct may be used by itself or may be used in conjunction with a vector. If a vector is used, then the choice of vector is dependent upon the method that will be used to transform host cells as is well known to those skilled in the art. For example, a plasmid vector can be used. The skilled artisan is well aware of the genetic elements that must be present on the vector in order to successfully transform, select and propagate host cells. The skilled artisan will also recognize that different independent transformation events may result in different levels and patterns of expression (Jones et al., (1985) EMBO J 4:241 1-2418; De Almeida et al., (1989) Mol Gen Genetics 218:78-86), and thus that multiple events are typically screened in order to obtain lines displaying the desired expression level and pattern. Such screening may be accomplished standard molecular biological, biochemical, and other assays including Southern analysis of DNA, Northern analysis of mRNA expression, PCR, real time quantitative PCR (qPCR), reverse transcription PCR (RT-PCR), immunoblotting analysis of protein expression, enzyme or activity assays, and/or phenotypic analysis.

As used herein, the term “expression” refers to the production of a functional end-product (e.g., an mRNA, guide RNA, or a protein) in either precursor or mature form.

As used herein, the term “introduced” means providing a nucleic acid (e.g., expression construct) or protein into a cell. Introduced includes reference to the incorporation of a nucleic acid into a eukaryotic or prokaryotic cell where the nucleic acid may be incorporated into the genome of the cell, and includes reference to the transient provision of a nucleic acid or protein to the cell. Introduced includes reference to stable or transient transformation methods, as well as sexually crossing. Thus, “introduced” in the context of inserting a nucleic acid fragment (e.g., a recombinant DNA construct/expression construct) into a cell, means “transfection” or “transformation” or “transduction” and includes reference to the incorporation of a nucleic acid fragment into a eukaryotic or prokaryotic cell where the nucleic acid fragment may be incorporated into the genome of the cell (e.g., nuclear chromosome, plasmid, plastid, chloroplast, or mitochondrial DNA), converted into an autonomous replicon, or transiently expressed (e.g., transfected mRNA).

As used herein, the term “mature” protein refers to a post-translationally processed polypeptide (i.e., one from which any pre- or propeptides present in the primary translation product have been removed). “Precursor” protein refers to the primary product of translation of mRNA (i.e., with pre- and propeptides still present). Pre- and propeptides may be but are not limited to intracellular localization signals.

As used herein, the term “stable transformation” refers to the transfer of a nucleic acid fragment into a genome of a host organism, including both nuclear and organellar genomes, resulting in genetically stable inheritance. In contrast, “transient transformation” refers to the transfer of a nucleic acid fragment into the nucleus, or other DNA-containing organelle, of a host organism resulting in gene expression without integration or stable inheritance. Host organisms containing the transformed nucleic acid fragments are referred to as “transgenic” organisms. The commercial development of genetically improved germplasm has also advanced to the stage of introducing multiple traits into crop plants, often referred to as a gene stacking approach. In this approach, multiple genes conferring different characteristics of interest can be introduced into a plant. Gene stacking can be accomplished by many means including but not limited to cotransformation, retransformation, and crossing lines with different genes of interest.

As used herein, the terms “crossed” or “cross” or “crossing” means the fusion of gametes via pollination to produce progeny (i.e., cells, seeds, or plants). The term encompasses both sexual crosses (the pollination of one plant by another) and selfing (self-pollination, i.e., when the pollen and ovule (or microspores and megaspores) are from the same plant or genetically identical plants).

As used herein, the term “introgression” refers to the transmission of a desired allele of a genetic locus from one genetic background to another. For example, introgression of a desired allele at a specified locus can be transmitted to at least one progeny plant via a sexual cross between two parent plants, where at least one of the parent plants has the desired allele within its genome. Alternatively, for example, transmission of an allele can occur by recombination between two donor genomes, e.g., in a fused protoplast, where at least one of the donor protoplasts has the desired allele in its genome. The desired allele can be, e.g., a transgene, a modified (mutated or edited) native allele, or a selected allele of a marker or QTL.

As used herein, the term “hybridized” means hybridizing under conventional conditions, as described in Sambrook et al. (1989), preferably under stringent conditions. Stringent hybridization conditions are for example and not limitation: hybridizing in 4×SSC at 65° C. and subsequent multiple washing in 0.1×SSC at 65° C. for a total of approximately one hour. Less stringent hybridization conditions are for example and not limitation: hybridizing in 4×SSC at 37° C. and subsequent multiple washing in 1×SSC at room temperature. “Stringent hybridization conditions” can also mean for example and not limitation: hybridizing at 68° C. in 0.25 M sodiumphosphate, pH 7.2, 7% SDS, 1 mM EDTA and 1% BSA for 16 hours and subsequent two times washing with 2×SSC and 0.1% SDS at 68° C.

### Argonaute Endonucleases of the Invention

Argonaute may introduce double-stranded breaks or single-stranded breaks in the target nucleic acid, (e.g. genomic DNA). The double-stranded break can stimulate a cell's endogenous DNA-repair pathways (e.g., HR, NHEJ, A-NHEJ, or MMEJ). NHEJ can repair cleaved target nucleic acid without the need for a homologous template. This can result in deletions of the target nucleic acid. Homologous recombination (HR) can occur with a homologous template. The homologous template can comprise sequences that are homologous to sequences flanking the target nucleic acid cleavage site. After a target nucleic acid is cleaved by an Argonaute, the site of cleavage can be destroyed (e.g., the site may not be accessible for another round of cleavage with the original nucleic acid-targeting nucleic acid and Argonaute).

Argonaute proteins which can function as endonucleases can comprise three key functional domains: a PIWI endonuclease domain, a PAZ domain, and a MID domain. The PIWI domain may resemble a nuclease. The nuclease may be an RNase H or a DNA-guided ribonuclease. The PIWI domain may share a divalent cation-binding motif for catalysis exhibited by other nucleases that can cleave RNA and DNA. The divalent cation-binding motif may contain four negatively charged, evolutionary conserved amino acids. The four negatively charged evolutionary conserved amino acids may be aspartate-glutamate-aspartate-aspartate (DEDD). The four negatively charged evolutionary conserved amino acids may form a catalytic tetrad that binds two Mg2+ ions and cleaves a target nucleic acid into products bearing a 3′ hydroxyl and 5′ phosphate group. The PIWI domain may further comprise one or more amino acids selected from a basic residue. The PIWI domain may further comprise one or more amino acids selected from histidine, arginine, lysine and a combination thereof. The histidine, arginine and/or lysine may play an important role in catalysis and/or cleavage. Cleavage of the target nucleic acid by Argonaute can occur at a single phosphodiester bond.

In some instances, one or more magnesium and/or manganese cations can facilitate target nucleic acid cleavage, wherein a first cation can nucleophilically attack and activate a water molecule and a second cation can stabilize the transition state and leaving group.

The MID domain can bind the 5′ phosphate and first nucleotide of the designed nucleic acid-targeting nucleic acid. The PAZ domain can use its oligonucleotide-binding fold to secure the 3′ end of the designed nucleic acid-targeting nucleic acid.

The Argonaute protein may comprise one or more domains. The Argonaute protein may comprise a domain selected from a PAZ domain, a MID domain, and a PIWI domain or any combination thereof. The Argonaute protein may comprise a domain architecture of N-PAZ-MID-PIWI-C. The PAZ domain may comprise an oligonucleotide-binding fold to secure a 3′ end of a nucleic acid-targeting nucleic acid. Release of the 3′-end of the nucleic acid-targeting nucleic acid from the PAZ domain may facilitate the transitioning of the Argonaute ternary complex into a cleavage active conformation. The MID domain may bind a 5′ phosphate and a first nucleotide of the nucleic acid-targeting nucleic acid. The target nucleic acid can remain bound to the Argonaute through many rounds of cleavage by means of anchorage of the 5′ phosphate in the MID domain.

An Argonaute can comprise a nucleic acid-binding domain. The nucleic acid-binding domain can comprise a region that contacts a nucleic acid. A nucleic acid-binding domain can comprise a nucleic acid. A nucleic acid-binding domain can comprise a proteinaceous material. A nucleic acid-binding domain can comprise nucleic acid and a proteinaceous material. A nucleic acid-binding domain can comprise DNA. A nucleic acid-binding domain can comprise single-stranded DNA. Examples of nucleic acid-binding domains can include, but are not limited to, a helix-turn-helix domain, a zinc finger domain, a leucine zipper (bZIP) domain, a winged helix domain, a winged helix turn helix domain, a helix-loop-helix domain, a HMG-box domain, a Wor3 domain, an immunoglobulin domain, a B3 domain, and a TALE domain. A nucleic acid-binding domain can be a domain of an Argonaute protein. An Argonaute protein can be a eukaryotic Argonaute or a prokaryotic Argonaute. An Argonaute protein can bind RNA or DNA, or both RNA and DNA. An Argonaute protein can cleave RNA, or DNA, or both RNA and DNA. In some instances, an Argonaute protein binds a DNA and cleaves the DNA. In some instances, the Argonaute protein binds a double-stranded DNA and cleaves a double-stranded DNA. In some instances, two or more nucleic acid-binding domains can be linked together. Linking a plurality of nucleic acid-binding domains together can provide increased polynucleotide targeting specificity. Two or more nucleic acid-binding domains can be linked via one or more linkers. The linker can be a flexible linker. Linkers can comprise 1, 2, 3, 4, 5, 6, 7, 8, 9, 10, 11, 12, 13, 14, 15, 16, 17, 18, 19, 20, 21, 22, 23, 24, 25, 30, 35, 40 or more amino acids in length. The linker domain may comprise glycine and/or serine, and in some embodiments may consist of or may consist essentially of glycine and/or serine. Linkers can be a nucleic acid linker which can comprise nucleotides. A nucleic acid linker can link two DNA-binding domains together. A nucleic acid linker can be at most 5, 10, 15, 20, 25, 30, 35, 40, 45, or 50 or more nucleotides in length. A nucleic acid linker can be at least 5, 10, 15, 30, 35, 40, 45, or 50 or more nucleotides in length.

Nucleic acid-binding domains can bind to nucleic acid sequences. Nucleic acid binding domains can bind to nucleic acids through hybridization. Nucleic acid-binding domains can be engineered (e.g., engineered to hybridize to a sequence in a genome). A nucleic acid-binding domain can be engineered by molecular cloning techniques (e.g., directed evolution, site-specific mutation, and rational mutagenesis).

An Argonaute can comprise a nucleic acid-cleaving domain. The nucleic acid-cleaving domain can be a nucleic acid-cleaving domain from any nucleic acid-cleaving protein. The nucleic acid-cleaving domain can originate from a nuclease. Suitable nucleic acid-cleaving domains include the nucleic acid-cleaving domain of endonucleases (e.g., AP endonuclease, RecBCD enonuclease, T7 endonuclease, T4 endonuclease IV, Bal 31 endonuclease, EndonucleaseI (endo I), Micrococcal nuclease, Endonuclease II (endo VI, exo III)), exonucleases, restriction nucleases, endoribonucleases, exoribonucleases, RNases (e.g., RNAse I, II, or III). A nucleic acid-binding domain can be a domain of an Argonaute protein. An Argonaute protein can be a eukaryotic Argonaute or a prokaryotic Argonaute. An Argonaute protein can bind RNA or DNA, or both RNA and DNA. An Argonaute protein can cleave RNA, or DNA, or both RNA and DNA. In some instances, an Argonaute protein binds a DNA and cleaves the DNA. In some instances, the Argonaute protein binds a double-stranded DNA and cleaves a double-stranded DNA. In some instances, the nucleic acid-cleaving domain can originate from the FokI endonuclease. An Argonaute can comprise a plurality of nucleic acid-cleaving domains. Nucleic acid-cleaving domains can be linked together. Two or more nucleic acid-cleaving domains can be linked via a linker. In some embodiments, the linker can be a flexible linker as described herein. Linkers can comprise 1, 2, 3, 4, 5, 6, 7, 8, 9, 10, 11, 12, 13, 14, 15, 16, 17, 18, 19, 20, 21, 22, 23, 24, 25, 30, 35, 40 or more amino acids in length. In some embodiments, an Argonaute can comprise the plurality of nucleic acid-cleaving domains.

Argonautes can introduce double-stranded breaks or single-stranded breaks in nucleic acid, (e.g., genomic DNA). The double-stranded break can stimulate a cell's endogenous DNA-repair pathways (e.g. homologous recombination and non-homologous end joining (NHEJ) or alternative non-homologues end joining (A-NHEJ)). NHEJ can repair cleaved target nucleic acid without the need for a homologous template. This can result in deletions of the target nucleic acid. Homologous recombination (HR) can occur with a homologous template. The homologous template can comprise sequences that are homologous to sequences flanking the target nucleic acid cleavage site. After a target nucleic acid is cleaved by an Argonaute the site of cleavage can be destroyed (e.g., the site may not be accessible for another round of cleavage with the original nucleic acid-targeting nucleic acid and Argonaute).

In some cases, homologous recombination can insert an exogenous polynucleotide sequence into the target nucleic acid cleavage site. An exogenous polynucleotide sequence can be called a donor polynucleotide. In some instances of the methods of the disclosure the donor polynucleotide, a portion of the donor polynucleotide, a copy of the donor polynucleotide, or a portion of a copy of the donor polynucleotide can be inserted into the target nucleic acid cleavage site. A donor polynucleotide can be an exogenous polynucleotide sequence. A donor polynucleotide can be a sequence that does not naturally occur at the target nucleic acid cleavage site. A vector can comprise a donor polynucleotide. The modifications of the target DNA due to NHEJ and/or HR can lead to, for example, mutations, deletions, alterations, integrations, gene correction, gene replacement, gene tagging, transgene insertion, nucleotide deletion, gene disruption, and/or gene mutation. The process of integrating non-native nucleic acid into genomic DNA can be referred to as genome engineering.

In some cases, the Argonaute can comprise an amino acid sequence having at most 10%, at most 15%, at most 20%, at most 30%, at most 40%, at most 50%, at most 60%, at most 70%, at most 75%, at most 80%, at most 85%, at most 90%, at most 95%, at most 99%, or 100%, amino acid sequence identity to a wild type exemplary Argonaute (e.g., NgAgo).

In some cases, the Argonaute can comprise an amino acid sequence having at least 10%, at least 15%, 20%, at least 30%, at least 40%, at least 50%, at least 60%, at least 70%, at least 75%, at least 80%, at least 85%, at least 90%, at least 95%, at least 99%, or 100%, amino acid sequence identity to a wild type exemplary Argonaute (e.g., NgAgo).

In some cases, the Argonaute can comprise an amino acid sequence having at most 10%, at most 15%, at most 20%, at most 30%, at most 40%, at most 50%, at most 60%, at most 70%, at most 75%, at most 80%, at most 85%, at most 90%, at most 95%, at most 99%, or 100%, amino acid sequence identity to the nuclease domain of a wild type exemplary Argonaute (e.g., NgAgo).

An Argonaute can comprise at least 70, 75, 80, 85, 90, 95, 97, 99, or 100% identity to wild-type Argonaute (e.g., NgAgo) over 10 contiguous amino acids of the MID domain. An Argonaute can comprise at most 70, 75, 80, 85, 90, 95, 97, 99, or 100% identity to wild-type Argonaute (e.g., NgAgo) over 10 contiguous amino acids of the MID domain. An Argonaute can comprise at least 70, 75, 80, 85, 90, 95, 97, 99, or 100% identity to wild-type Argonaute (e.g., NgAgo) over 10 contiguous amino acids of the PAZ domain. An Argonaute can comprise at most 70, 75, 80, 85, 90, 95, 97, 99, or 100% identity to wild-type Argonaute (e.g., NgAgo) over 10 contiguous amino acids of the PAZ domain. An Argonaute can comprise at least 70, 75, 80, 85, 90, 95, 97, 99, or 100% identity to wild-type Argonaute (e e.g., NgAgo) over 10 contiguous amino acids of the PIWI domain. An Argonaute can comprise at most 70, 75, 80, 85, 90, 95, 97, 99, or 100% identity to wild-type Argonaute (e.g., NgAgo) over 10 contiguous amino acids of the PIWI domain.

The Argonaute proteins disclosed herein may comprise one or more modifications. The modification may comprise a post-translational modification. The modification of the target nucleic acid may occur at least 1, 2, 3, 4, 5, 6, 7, 8, 9, 10, 20, 30, 40, 50, 60, 70, 80, 90, 100 or more amino acids away from the either the carboxy terminus or amino terminus end of the Argonaute protein. The modification of the Argonaute protein may occur at most 1, 2, 3, 4, 5, 6, 7, 8, 9, 10, 20, 30, 40, 50, 60, 70, 80, 90, 100 or more amino acids away from the carboxy terminus or amino terminus end of the Argonaute protein. The modification may occur due to the modification of a nucleic acid encoding an Argonaute protein. Exemplary modifications can comprise methylation, demethylation, acetylation, deacetylation, ubiquitination, deubiquitination, deamination, alkylation, depurination, oxidation, pyrimidine dimer formation, transposition, recombination, chain elongation, ligation, glycosylation. Phosphorylation, dephosphorylation, adenylation, deadenylation, SUMOylation, deSUMOylation, ribosylation, deribosylation, myristoylation, remodelling, cleavage, oxidoreduction, hydrolation, and isomerization.

The Argonaute can comprise a modified form of a wild type exemplary Argonaute. The modified form of the wild type exemplary Argonaute can comprise an amino acid change (e.g., deletion, insertion, or substitution) that reduces the nucleic acid-cleaving activity of the Argonaute. Alternatively, the amino acid change can result in an increase in nucleic acid-cleaving activity of the Argonaute. Alternatively, the amino acid change can result in a change in the temperature at which the Argonaute is active.

The Argonaute protein may comprise one or more mutations. The Argonaute protein may comprise amino acid modifications (e.g., substitutions, deletions, additions, etc., and combinations thereof). The Argonaute protein may comprise one or more non-native sequences (e.g., a fusion, as defined herein). The amino acid modifications may comprise one or more non-native sequences (e.g., a fusion as defined herein, an affinity tag). The amino acid modifications may not substantially alter the activity of the endonuclease. The Argonaute comprising amino acid modifications and/or fusions may retain at least about 75%, at least about 80%, at least about 85%, at least about 90%, at least about 95%, at least about 97% or 100% activity of the wild-type Argonaute. Modifications (e.g., mutations) of the disclosure can be produced by site-directed mutation. Mutations can include substitutions, additions, and deletions, or any combination thereof. In some instances, the mutation converts the mutated amino acid to alanine. In some instances, the mutation converts the mutated amino acid to another amino acid (e.g., glycine, serine, threonine, cysteine, valine, leucine, isoleucine, methionine, proline, phenylalanine, tyrosine, tryptophan, aspartic acid, glutamic acid, asparagines, glutamine, histidine, lysine, or arginine). The mutation can convert the mutated amino acid to a non-natural amino acid (e.g., selenomethionine). The mutation can convert the mutated amino acid to amino acid mimics (e.g., phosphomimics). The mutation can be a conservative mutation. For example, the mutation can convert the mutated amino acid to amino acids that resemble the size, shape, charge, polarity, conformation, and/or rotamers of the mutated amino acids (e.g., cysteine/serine mutation, lysine/asparagine mutation, histidine/phenylalanine mutation).

In some instances, the Argonaute can target nucleic acid. The Argonaute can target DNA. In some instances, the Argonaute is modified to express nickase activity. In some instances, the Argonaute is modified to target nucleic acid but is enzymatically inactive (e.g., does not have endonuclease or nickase activity). In some instances, the Argonaute is modified to express one or more of the following activities, with or without endonuclease activity: nickase, exonuclease, DNA repair (e.g., DNA DSB repair), helicase, transcriptional (co-)activation, transcriptional (co-) repression, methylase, and/or demethylase.

In some instances, the Argonaute is active at temperatures suitable for growth and culture of plants and plant cells, such as for example and not limitation, about 20° C. to about 35° C., preferably about 23° C. to about 32° C., and most preferably about 25° C. to about 28° C.

The Argonaute can comprise one or more non-native sequences (e.g., a fusion as discussed herein). In some instances, the non-native sequence of the Argonaute comprises a moiety that can alter transcription. Transcription can be increased or decreased. Transcription can be altered by at least about 1-fold, 2-fold, 3-fold, 4-fold, 5-fold, 10-fold, 15-fold, or 20-fold or more. Transcription can be altered by at most about 1-fold, 2-fold, 3-fold, 4-fold, 5-fold, 10-fold, 15-fold, or 20-fold or more. The moiety can be a transcription factor. When an Argonaute is a fusion Argonaute comprising a non-native sequence that can alter transcription, the Argonaute may comprise reduced enzymatic activity as compared to a wild-type Argonaute.

By way of non-limiting example, Argonaute may bind a nucleic acid-targeting nucleic acid (e.g., single-stranded DNA, single-stranded RNA) that guides it to a target nucleic acid that is complementary to the nucleic acid-targeting nucleic acid, wherein the target nucleic acid comprises a dsDNA (e.g., such as a plasmid, genomic DNA, etc.), and thereby carries out site specific cleavage within the target nucleic acid.

In some embodiments of the invention, the methods and compositions comprise NgAgo, and said methods and compositions are used at temperatures suitable for growth and culture of plants and plant cells, such as for example and not limitation, about 20° C. to about 35° C., preferably about 23° C. to about 32° C., and most preferably about 25° C. to about 28° C.

In some embodiments of the invention, the Argonaute is provided separately from the nucleic acid-targeting nucleic acid. In other embodiments, the Argonaute is provided in a complex wherein the nucleic acid-targeting nucleic acid is pre-associated with the Argonaute.

In some embodiments of the invention, the Argonaute is provided as part of an expression cassette on a suitable vector, configured for expression of the Argonaute in a desired host cell (e.g., a plant cell or a plant protoplast). The vector may allow transient expression of the Argonaute. Alternatively, the vector may allow the expression cassette and/or Argonaute to be stably maintained in the host cell, such as for example and not limitation, by integration into the host cell genome, including stable integration into the genome. In some embodiments, the host cell is an ancestral cell, thereby providing heritable expression of the Argonaute. The Argonaute contained in the expression cassette may be a heterologous polypeptide as described below.

In other embodiments, the Argonaute is provided as a heterologous polypeptide, either alone or as a transcriptional or translational fusion (to either or both of the N-terminal and C-terminal domains of the Argonaute), as discussed herein, with one or more functional domains, such as for example and not limitation, a localization signal (e.g., nuclear localization signal, chloroplast localization signal), an epitope tag, an antibody, and/or a functional protein, such as for example and not limitation, a reporter protein (e.g., a fluorescent reporter protein such as mNeonGreen and GFP), proteins involved in DNA break repair (e.g., DNA DSBs), a nickase, a helicase, an exonuclease, a transcriptional (co-) activator, a transcriptional (co-) repressor, a methylase, and/or a demethylase.

In other embodiments, the Argonaute is provided as a protein. In still other embodiments, the Argonaute is provided as a nucleic acid, such as for example and not limitation, an mRNA.

In any of the above embodiments, the Argonaute may be optimized for expression in plants, including but not limited to plant-preferred promoters, plant tissue-specific promoters, and/or plant-preferred codon optimization, as discussed in more detail herein.

In any of the above embodiments, the Argonaute may be present as a fusion (e.g., transcriptional and/or translational fusion) with polynucleotides or polypeptides of interest that are associated with certain plant genes and/or traits. Such plant genes and/or traits include for example and not limitation, an acetolactate synthase (ALS) gene, an acetohydroxyacid synthase (AHAS) gene, an enolpyruvylshikimate phosphate synthase gene (EPSPS) gene, a male fertility gene (e.g., MS45, MS26 or MSCA1), a herbicide resistance gene, a male sterility gene, a female fertility gene, a female sterility gene, a male or female restorer gene, and genes associated with the traits of sterility, fertility, herbicide resistance, herbicide tolerance, biotic stress such as fungal resistance, viral resistance, or insect resistance, abiotic stress such as drought tolerance, chilling tolerance, or cold tolerance, nitrogen use efficiency, phosphorus use efficiency, water use efficiency and crop or biomass yield (e.g., improved or decreased crop or biomass yield), and mutants of such genes. Such mutants include, for example and not limitation, amino acid substitutions, deletions, insertions, codon optimization, and regulatory sequence changes to alter the gene expression profiles.

### Nucleic Acid-Targeting Nucleic Acids (Nucleic Acid-Targeting Guide Nucleic Acids) of the Invention

Disclosed herein are nucleic acid-targeting nucleic acids (nucleic acid-targeting guide nucleic acids) that can direct the activities of an associated polypeptide (e.g., Argonaute protein) to a specific target sequence within a target nucleic acid. The nucleic acid-targeting nucleic acid can comprise nucleotides. The nucleic acid-targeting nucleic acid may be a single-stranded DNA (ssDNA). The nucleic acid-targeting nucleic acid may comprise double-stranded DNA. The nucleic acid-targeting nucleic acid may comprise single or double-stranded RNA.

A nucleic acid-targeting nucleic acid can comprise one or more modifications (e.g., a base modification, a backbone modification), to provide the nucleic acid with a new or enhanced feature (e.g., improved stability). The one or more modifications may, in addition to or independently of improving stability, change the binding specificity of the nucleic acid-targeting nucleic acid in a user-preferred way (e.g., greater or lesser specificity or tolerance or lack of tolerance for a specific mismatch). The one or more modifications, whether to improve stability or alter binding specificity or both, preserve the ability of the nucleic acid-targeting nucleic acid to interact with both Argonaute and the target nucleic acid. A nucleic acid-targeting nucleic acid can comprise a nucleic acid affinity tag. A nucleoside can be a base-sugar combination. The base portion of the nucleoside can be a heterocyclic base. The two most common classes of such heterocyclic bases are the purines and the pyrimidines. Nucleotides can be nucleosides that further include a phosphate group covalently linked to the sugar portion of the nucleoside. For those nucleosides that include a pentofuranosyl sugar, the phosphate group can be linked to the 2′, the 3′, or the 5′ hydroxyl moiety of the sugar. In forming nucleic acid-targeting nucleic acids, the phosphate groups can covalently link adjacent nucleosides to one another to form a linear polymeric compound. In turn, the respective ends of this linear polymeric compound can be further joined to form a circular compound; however, linear compounds are generally suitable. In addition, linear compounds may have internal nucleotide base complementarity and may therefore fold in a manner as to produce a fully or partially double-stranded compound. Within nucleic acid-targeting nucleic acids, the phosphate groups can commonly be referred to as forming the internucleoside backbone of the nucleic acid-targeting nucleic acid. The linkage or backbone of the nucleic acid-targeting nucleic acid can be a 3′ to 5′ phosphodiester linkage.

The nucleic acid-targeting nucleic acid can be a dsRNA or a ssRNA or a dsDNA or a ssDNA. In a preferred embodiment, the nucleic acid-targeting nucleic acid is a short ssDNA. In some embodiments, the ssDNA is 50 nucleotides or less in length, preferably 40 nucleotides or less in length, and most preferably 30 nucleotides or less in length. In a particularly preferred embodiment, the nucleic acid-targeting nucleic acid is a 5′-phosphorylated ssDNA of 20, 21, 22, 23, 24, 25, 26, 27, 28, 29, or 30 nucleotides in length.

Many modifications of synthesized DNA oligonucleotides are commercially available and can be useful for stabilizing the oligonucleotide in a host cell to prolong its availability for use by the Argonaute endonuclease in gene editing. Non-limiting examples of modifications that can be used to increase stability include a modified backbone and/or modified internucleoside linkages. Non-limiting examples of such modifications include locked nucleic acid (LNA) bases, internucleotide phosphorothioate bonds in the backbone, 2′-O-Methyl RNA bases, unlocked nucleic acid (UNA) bases, or inverted dT at the 3′ end. Other modifications can be made to increase or decrease the tolerance of the guide-DNA for mismatches with the target site, either to increase or decrease the specificity of the endonuclease complex as needed to achieve the desired gene goals. Non-limiting examples of modifications that can be used to affect targeting specificity are the addition of 5-Methyl dC, 5-hydroxybutynl-2′-deoxyuridine, 5-Nitroindole, or deoxyInosine. Still other modifications can be made to prevent unwanted integration of the guide-DNA into the host cell genome. Non-limiting examples are use of an Inverted Dideoxy-T at the 5′ end to prevent ligation into the genome or use of Inverted dT or Dideoxycytidine at the 3′ end to prevent extension due to DNA polymerases.

Modified backbones can include those that retain a phosphorus atom in the backbone and those that do not have a phosphorus atom in the backbone. Suitable modified nucleic acid-targeting nucleic acid backbones containing a phosphorus atom therein can include, for example, phosphorothioates, chiral phosphorothioates, phosphorodithioates, phosphotriesters, aminoalkylphosphotriesters, methyl and other alkyl phosphonates such as 3′-alkylene phosphonates, 5′-alkylene phosphonates, chiral phosphonates, phosphinates, phosphoramidates including 3′-amino phosphoramidate and aminoalkylphosphoramidates, phosphorodiamidates, thionophosphoramidates, thionoalkylphosphonates, thionoalkylphosphotriesters, selenophosphates, and boranophosphates having normal 3′-5′ linkages, 2′-5′ linked analogs, and those having inverted polarity wherein one or more internucleotide linkages is a 3′ to 3′, a 5′ to 5′ or a 2′ to 2′ linkage. Suitable nucleic acid-targeting nucleic acids having inverted polarity can comprise a single 3′ to 3′ linkage at the 3′-most internucleotide linkage (i.e. a single inverted nucleoside residue in which the nucleobase is missing or has a hydroxyl group in place thereof). Various salts (e.g., potassium chloride or sodium chloride), mixed salts, and free acid forms can also be included. A nucleic acid-targeting nucleic acid can comprise one or more phosphorothioate and/or heteroatom internucleoside linkages. A nucleic acid-targeting nucleic acid can comprise a morpholino backbone structure. For example, a nucleic acid can comprise a 6-membered morpholino ring in place of a ribose ring. In some of these embodiments, a phosphorodiamidate or other non-phosphodiester internucleoside linkage can replace a phosphodiester linkage. A nucleic acid-targeting nucleic acid can comprise polynucleotide backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These can include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; riboacetyl backbones; alkene containing backbones; sulfamate backbones; methyleneimino and methylenehydrazino backbones; sulfonate and sulfonamide backbones; amide backbones; and others having mixed N, O, S and CH₂ component parts.

A nucleic acid-targeting nucleic acid can comprise a nucleic acid mimetic. The term “mimetic” can be intended to include polynucleotides wherein only the furanose ring or both the furanose ring and the internucleotide linkage are replaced with non-furanose groups, replacement of only the furanose ring can also be referred as being a sugar surrogate. The heterocyclic base moiety or a modified heterocyclic base moiety can be maintained for hybridization with an appropriate target nucleic acid. One such nucleic acid can be a peptide nucleic acid (PNA). In a PNA, the sugar-backbone of a polynucleotide can be replaced with an amide containing backbone, in particular an aminoethylglycine backbone. The nucleotides can be retained and are bound directly or indirectly to aza nitrogen atoms of the amide portion of the backbone. The backbone in PNA compounds can comprise two or more linked aminoethylglycine units which gives PNA an amide containing backbone. The heterocyclic base moieties can be bound directly or indirectly to aza nitrogen atoms of the amide portion of the backbone.

A nucleic acid-targeting nucleic acid can comprise linked morpholino units (i.e. morpholino nucleic acid) having heterocyclic bases attached to the morpholino ring. Linking groups can link the morpholino monomeric units in a morpholino nucleic acid. Non-ionic morpholino-based oligomeric compounds can have less undesired interactions with cellular proteins. Morpholino-based polynucleotides can be nonionic mimics of nucleic acid-targeting nucleic acids. A variety of compounds within the morpholino class can be joined using different linking groups. A further class of polynucleotide mimetic can be referred to as cyclohexenyl nucleic acids (CeNA). The furanose ring normally present in a nucleic acid molecule can be replaced with a cyclohexenyl ring. CeNA DMT (dimethoxytrityl) protected phosphoramidite monomers can be prepared and used for oligomeric compound synthesis using phosphoramidite chemistry. The incorporation of CeNA monomers into a nucleic acid chain can increase the stability of a DNA/RNA hybrid. CeNA oligoadenylates can form complexes with nucleic acid complements with similar stability to the native complexes. A further modification can include LNAs in which the 2′-hydroxyl group is linked to the 4′ carbon atom of the sugar ring thereby forming a 2′-C,4′-C-oxymethylene linkage thereby forming a bicyclic sugar moiety. The linkage can be a methylene (—CH₂—), group bridging the 2′ oxygen atom and the 4′ carbon atom wherein n is 1 or 2. LNA and LNA analogs can display very high duplex thermal stabilities with complementary nucleic acid (Tm=+3 to +10° C.), stability towards 3′-exonucleolytic degradation and good solubility properties.

A nucleic acid-targeting nucleic acid can comprise one or more substituted sugar moieties. Suitable polynucleotides can comprise a sugar substituent group selected from: OH; F; O-, S-, or N-alkyl; O-, S-, or N-alkenyl; O-, S- or N-alkynyl; or O-alkyl-O-alkyl, wherein the alkyl, alkenyl and alkynyl may be substituted or unsubstituted C₁ to C₁₀ alkyl or C₂ to C₁₀ alkenyl and alkynyl. Particularly suitable are O((CH₂)ₙO)ₘCH₃, O(CH₂)ₙOCH₃, O(CH₂)ₙNH₂, O(CH₂)ₙCH₃, O(CH₂)ₙONH₂, and O(CH₂)ₙON((CH₂)ₙCH₃)₂, where n and m are from 1 to about 10. A sugar substituent group can be selected from: C1 to C10 lower alkyl, substituted lower alkyl, alkenyl, alkynyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of a nucleic acid-targeting nucleic acid, or a group for improving the pharmacodynamic properties of a nucleic acid-targeting nucleic acid, and other substituents having similar properties. A suitable modification can include 2′-methoxyethoxy (2′-O—CH₂CH₂OCH₃, also known as 2′-O-(2-methoxyethyl) or 2′-MOE i.e., an alkoxyalkoxy group). A further suitable modification can include 2′-dimethylaminooxyethoxy, (i.e., a O(CH₂)₂ON(CH₃)₂ group, also known as 2′-DMAOE), and 2′-dimethylaminoethoxyethoxy (also known as 2′-O-dimethyl-amino-ethoxy-ethyl or 2′-DMAEOE), i.e., 2′-O—CH₂—O—CH₂—N(CH₃)₂. Other suitable sugar substituent groups can include methoxy (—O—CH₃), aminopropoxy (—O CH₂CH₂CH₂NH₂), allyl (—CH₂—CH═C—), —O-allyl (—O— CH₂—CH═CH₂) and fluoro (F). 2′-sugar substituent groups may be in the arabino (up) position or ribo (down) position. A suitable 2′-arabino modification is 2′-F. Similar modifications may also be made at other positions on the oligomeric compound, particularly the 3′ position of the sugar on the 3′ terminal nucleoside or in 2′-5′ linked nucleotides and the 5′ position of 5′ terminal nucleotide. Oligomeric compounds may also have sugar mimetics such as cyclobutyl moieties in place of the pentofuranosyl sugar.

A nucleic acid-targeting nucleic acid may also include nucleobase (often referred to simply as “base”) modifications or substitutions. As used herein, “unmodified” or “natural” nucleobases can include the purine bases, (e.g. adenine (A) and guanine (G)), and the pyrimidine bases, (e.g. thymine (T), cytosine (C) and uracil (U)). Modified nucleobases can include other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl (—C═C—CH₃) uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-aminoadenine, 8-azaguanine and 8-azaadenine, 7-deazaguanine and 7-deazaadenine and 3-deazaguanine and 3-deazaadenine. Modified nucleobases can include tricyclic pyrimidines such as phenoxazine cytidine (1H-pyrimido(5,4-b)(1,4)benzoxazin-2(3H)-one), phenothiazine cytidine (1H-pyrimido(5,4-b)(1,4)benzothiazin-2(3H)-one), G-clamps such as a substituted phenoxazine cytidine (e.g. 9-(2-aminoethoxy)-H-pyrimido(5,4-(b) (1,4)benzoxazin-2(3H)-one), carbazole cytidine (2H-pyrimido(4,5-b)indol-2-one), pyridoindole cytidine (Hpyrido(3′,2′:4,5)pyrrolo(2,3-d)pyrimidin-2-one).

Heterocyclic base moieties can include those in which the purine or pyrimidine base is replaced with other heterocycles, for example 7-deaza-adenine, 7-deazaguanosine, 2-aminopyridine and 2-pyridone. Nucleobases can be useful for increasing the binding affinity of a polynucleotide compound. These can include 5-substituted pyrimidines, 6-azapyrimidines and N-2, N-6 and O-6 substituted purines, including 2-aminopropyladenine, 5-propynyluracil and 5-propynylcytosine. 5-methylcytosine substitutions can increase nucleic acid duplex stability by 0.6-1.2° C. and can be suitable base substitutions (e.g., when combined with 2′-O-methoxyethyl sugar modifications).

A modification of a nucleic acid-targeting nucleic acid can comprise chemically linking to the nucleic acid-targeting nucleic acid one or more moieties or conjugates that can enhance the activity, cellular distribution or cellular uptake of the nucleic acid-targeting nucleic acid. These moieties or conjugates can include conjugate groups covalently bound to functional groups such as primary or secondary hydroxyl groups. Conjugate groups can include, but are not limited to, intercalators, reporter molecules, polyamines, polyamides, polyethylene glycols, polyethers, groups that enhance the pharmacodynamic properties of oligomers, and groups that can enhance the pharmacokinetic properties of oligomers. Conjugate groups can include, but are not limited to, cholesterols, lipids, phospholipids, biotin, phenazine, folate, phenanthridine, anthraquinone, acridine, fluoresceins, rhodamines, coumarins, and dyes. Groups that enhance the pharmacodynamic properties include groups that improve uptake, enhance resistance to degradation, and/or strengthen sequence-specific hybridization with the target nucleic acid. Groups that can enhance the pharmacokinetic properties include groups that improve uptake, distribution, metabolism or excretion of a nucleic acid. Conjugate moieties can include but are not limited to lipid moieties such as a cholesterol moiety, cholic acid a thioether, (e.g., hexyl-S-tritylthiol), a thiocholesterol, an aliphatic chain (e.g., dodecandiol or undecyl residues), a phospholipid (e.g., di-hexadecyl-rac-glycerol or triethylammonium 1,2-di-O-hexadecyl-rac-glycero-3-H-phosphonate), a polyamine or a polyethylene glycol chain, or adamantane acetic acid, a palmityl moiety, or an octadecylamine or hexylamino-carbonyl-oxycholesterol moiety. A modification may also include a “Protein Transduction Domain” or PTD (i.e., a cell penetrating peptide (CPP)). The PTD can refer to a polypeptide, polynucleotide, carbohydrate, or organic or inorganic compound that facilitates traversing a lipid bilayer, micelle, cell membrane, organelle membrane, or vesicle membrane. A PTD can be attached to another molecule, which can range from a small polar molecule to a large macromolecule and/or a nanoparticle, and can facilitate the molecule traversing a membrane, for example going from extracellular space to intracellular space, or cytosol to within an organelle. A PTD can be covalently linked to the amino terminus of a polypeptide. A PTD can be covalently linked to the carboxyl terminus of a polypeptide. A PTD can be covalently linked to a nucleic acid. Exemplary PTDs can include, but are not limited to, a minimal peptide protein transduction domain; a polyarginine sequence comprising a number of arginines sufficient to direct entry into a cell (e.g., 3, 4, 5, 6, 7, 8, 9, 10, or 10-50 arginines), a VP22 domain, polylysine, and transportan, arginine homopolymer of from 3 arginine residues to 50 arginine residues. The PTD can be an activatable CPP (ACPP). ACPPs can comprise a polycationic CPP (e.g., Arg9 or “R9”) connected via a cleavable linker to a matching polyanion (e.g., Glu9 or “E9”), which can reduce the net charge to nearly zero and thereby inhibits adhesion and uptake into cells. Upon cleavage of the linker, the polyanion can be released, locally unmasking the polyarginine and its inherent adhesiveness, thus “activating” the ACPP to traverse the membrane.

Still other modifications of a nucleic-acid targeting nucleic acid can comprise a 5′ cap, a 3′ polyadenylated tail, a riboswitch sequence, a stability control sequence, a sequence that forms a dsRNA duplex, a modification or sequence that targets the nucleic-acid targeting nucleic acid to a subcellular location, a modification or sequence that provides for tracking, a modification or sequence that provides a binding site for proteins, a 5-methyl dC nucleotide, a 2,6-Diaminopurine nucleotide, a 2′-Fluoro A nucleotide, a 2′-Fluoro U nucleotide; a 2′-O-Methyl RNA nucleotide, a phosphorothioate bond, linkage to a cholesterol molecule, linkage to a polyethylene glycol molecule, linkage to a spacer molecule, a 5′ to 3′ covalent linkage, or any combination thereof.

The nucleic acid-targeting nucleic acid can be at least about 5, 6, 7, 8, 9, 10, 11, 12, 13, 14, 15, 16, 17, 18, 19, 20, 21, 22, 23, 24, 25, 26, 27, 28, 29, or 30 or more nucleotides in length. The nucleic acid-targeting nucleic acid can be at most about 5, 6, 7, 8, 9, 10, 11, 12, 13, 14, 15, 16, 17, 18, 19, 20, 21, 22, 23, 24, 25, 26, 27, 28, 29, or 30 or more nucleotides in length. In some instances, the nucleic acid-targeting nucleic acid is 20, 21, 22, 23, 24, 25, 26, 27, 28, 29, or 30 nucleotides in length. In some instances, the nucleic acid-targeting nucleic acid is phosphorylated at either the 5′ or 3′ end, or both ends.

The nucleic acid-targeting nucleic acid can comprise a 5′ deoxycytosine. The nucleic acid-targeting nucleic acid can comprise a deoxycytosine-deoxyadenosine at the 5′ end of the nucleic acid-targeting nucleic acid. In some embodiments, any nucleotide can be present at the 5′ end, and/or can contain a modified backbone or other modifications as discussed herein. The nucleic acid-targeting nucleic acid may comprise a 5′ phosphorylated end.

The nucleic acid-targeting nucleic acid can be fully complementary to the target nucleic acid (e.g., hybridizable). The nucleic acid-targeting nucleic acid can be partially complementary to the target nucleic acid. For example, the nucleic acid-targeting nucleic acid can be at least 30, 40, 50, 60, 70, 80, 90, 95, or 100% complementary to the target nucleic acid over the region of the nucleic acid-targeting nucleic acid. The nucleic acid-targeting nucleic acid can be at most 30, 40, 50, 60, 70, 80, 90, 95, or 100% complementary to the target nucleic acid over the region of the nucleic acid-targeting nucleic acid.

A stretch of nucleotides of the nucleic acid-targeting nucleic acid can be complementary to the target nucleic acid (e.g., hybridizable). A stretch of at least 2, 3, 4, 5, 6, 7, 8, 9, 10, 11, 12, 13, 14, 15, 16, 17, 18, 19, 20, 21, 22, 23, 24, 25, 26, 27, 28, 29, or 30 contiguous nucleotides can be complementary to target nucleic acid. A stretch of at most 2, 3, 4, 5, 6, 7, 8, 9, 10, 11, 12, 13, 14, 15, 16, 17, 18, 19, 20, 21, 22, 23, 24, 25, 26, 27, 28, 29, or 30 contiguous nucleotides can be complementary to target nucleic acid.

A portion of the nucleic acid-targeting nucleic acid which is fully complementary to the target nucleic acid may extend from at least nucleotide 2, to nucleotide 17 (as counted from the 5′ end of the nucleic acid-targeting nucleic acid). A portion of the nucleic acid-targeting nucleic acid which is fully complementary to the target nucleic acid may extend from at least nucleotide 3 to nucleotide 20, nucleotide 4 to nucleotide 18, nucleotide 5 to nucleotide 16, nucleotide 6 to nucleotide 14, nucleotide 7 to nucleotide 12, nucleotide 6 to nucleotide 16, nucleotide 6 to nucleotide 18, or nucleotide 6 to nucleotide 20.

The nucleic acid-targeting nucleic acid can hybridize to a target nucleic acid. The nucleic acid-targeting nucleic acid can hybridize with a mismatch between the nucleic acid-targeting nucleic acid and the target nucleic acid (e.g., a nucleotide in the nucleic acid-targeting nucleic acid may not hybridize with the target nucleic acid). A nucleic acid-targeting nucleic acid can comprise at least 1, 2, 3, 4, 5, 6, 7, 8, 9, or 10 or more mismatches when hybridized to a target nucleic acid. A nucleic acid-targeting nucleic acid can comprise at most 1, 2, 3, 4, 5, 6, 7, 8, 9, or 10 or more mismatches when hybridized to a target nucleic acid.

The nucleic acid-targeting nucleic acid may direct cleavage of the target nucleic acid at the bond between the 1st and 2nd, 2nd and 3rd, 3rd and 4th, 4th and 5th, 5th and 6th, 6th and 7th, 7th and 8th, 8th and 9th, 9th and 10th, 10th and 11th, 11th and 12th, 12th and 13th, 13th and 14th, 14th and 15th, 15th and 16th, 16th and 17th, 17th and 18th, 18th and 19th, 19th and 20th, 20th and 21st, 21st and 22nd, 22nd and 23th, 23rd and 24th, or 24th and 25th nucleotides relative to the 5′-end of the designed nucleic acid-targeting nucleic acid. The designed nucleic acid-targeting nucleic acid may direct cleavage of the target nucleic acid at the bond between the 10th and 11th nucleotides (t10 and t11) relative to the 5′-end of the designed nucleic acid-targeting nucleic acid. The precise design for optimum cleavage of the target nucleic acid cleavage site may be determined by preliminary tests with plasmid targets incorporating the cleavage site.

As discussed herein, the nucleic acid-targeting nucleic acid can be a ds RNA or a ssRNA or a dsDNA or a ssDNA. In a preferred embodiment, the nucleic acid-targeting nucleic acid is a short ssDNA. In some embodiments, the ssDNA is 50 nucleotides or less in length, preferably 40 nucleotides or less in length, most preferably 30 nucleotides or less in length. In a particularly preferred embodiment, the nucleic acid-targeting nucleic acid is a 5′-phosphorylated ssDNA of 20, 21, 22, 23, 24, 25, 26, 27, 28, 29, or 30 nucleotides in length.

### Target Nucleic Acids of the Invention

The target nucleic acid may comprise one or more sequences that are at least partially complementary to one or more designed nucleic acid-targeting nucleic acids. The target nucleic acid can be part or all of a gene, a 5′ end of a gene, a 3′ end of a gene, a regulatory element (e.g. promoter, enhancer), a pseudogene, non-coding DNA, a microsatellite, an intron, an exon, chromosomal DNA, mitrochondrial DNA, sense DNA, antisense DNA, nucleoid DNA, chloroplast DNA, or RNA among other nucleic acid entities. The target nucleic acid can be part or all of a plasmid DNA. The plasmid DNA or a portion thereof may be negatively supercoiled. The target nucleic acid can be in vitro or in vivo.

The target nucleic acid may comprise a sequence within a low GC content region. The target nucleic acid may be negatively supercoiled. Thus, by non-limiting example, the target nucleic acid may comprise a GC content of at least about 5, 10, 15, 20, 25, 30, 35, 40, 45, 50, 55, 60, or 65% or more. The target nucleic acid may comprise a GC content of at most about 5, 10, 15, 20, 25, 30, 35, 40, 45, 50, 55, 60, or 65% or more.

A region comprising a particular GC content may be the length of the target nucleic acid that hybridizes with the designed nucleic acid-targeting nucleic acid. The region comprising the GC content may be longer or shorter than the length of the region that hybridizes with the designed nucleic acid-targeting nucleic acid. The region comprising the GC content may be at least 30, 40, 50, 60, 70, 80, 90 or 100 or more nucleotides longer or shorter than the length of the region that hybridizes with the designed nucleic acid-targeting nucleic acid. The region comprising the GC content may be at most 30, 40, 50, 60, 70, 80, 90 or 100 or more nucleotides longer or shorter than the length of the region that hybridizes with the designed nucleic acid-targeting nucleic acid.

In some embodiments, the target nucleic acid is found within a plant genome. The plant can be a monocot or a dicot. Non-limiting examples of monocots include of maize, rice, sorghum, rye, barley, wheat, millet, oats, sugarcane, turfgrass, or switchgrass. Non-limiting examples of dicots include soybean, canola, alfalfa, sunflower, cotton, tobacco, peanut, potato, winter oil seed rape, spring oil seed rape, sugar beet, fodder beet, red beet, sunflower, tobacco, Arabidopsis, or safflower. In some embodiments, the target nucleic acid comprises an acetolactate synthase (ALS) gene (including mutants thereof), an acetohydroxyacid synthase (AHAS) gene (including mutants thereof), an Enolpyruvylshikimate Phosphate Synthase Gene (EPSPS) gene (including mutants of the EPSPS gene such as for example and not limitation T102I/P106A, T102I/P106S, T102I/P106C, G101A/A192T, and G101A/A144D), a male fertility (MS45, MS26 or MSCA1) gene (including mutants thereof), a male sterility gene, a sterility restorer gene, a herbicide resistance gene, a herbicide tolerance gene, a fungal resistance gene, a viral resistance gene, an insect resistance gene, a gene associated with increased or decreased plant yield (e.g. biomass or seeds), a gene associated with drought, chilling or cold resistance/tolerance, with nitrogen, phosphorus or water use efficiency, or another target site described in WO2015/026883. The target nucleic acid may include genes associated with one or more of the following traits: herbicide resistance, herbicide tolerance, biotic stress resistance, fungal resistance, viral resistance, insect resistance, increased or decreased plant yield (e.g. biomass or seeds), abiotic stress resistance, nitrogen use efficiency, phosphorus use efficiency, water use efficiency, and drought resistance. The target nucleic acid may include mutations such as for example and not limitation, amino acid substitutions, deletions, insertions, codon optimization, and regulatory sequence changes to alter the gene expression profiles. The target nucleic acid may further include any of the nucleic acids for use with the invention as described hereinbelow.

Nucleic Acids/Polypeptides for Use with the Invention

Any nucleic acid of interest can be provided, integrated into the host cell genome (e.g., a plant cell or protoplast) at the target nucleic acid or transiently maintained within the host cell, and expressed in the host cell by using the invented methods and compositions. Such nucleic acid may be non-native. The nucleic acid of interest may include mutations such as for example and not limitation, amino acid substitutions, deletions, insertions, regulatory sequence changes to alter the gene expression profiles, transcriptional and/or translational fusions as discussed herein, and/or codon optimization. One or more nucleic acids of interest may be used in the methods and compositions described herein. The one or more nucleic acids may be present as a fusion (e.g., transcriptional and/or translational fusion) with Argonaute.

Nucleic acids/polypeptides of interest include, but are not limited to, herbicide-resistance coding sequences, herbicide-tolerance coding sequences, insecticidal/insect resistance coding sequences, nematicidal coding sequences, antimicrobial coding sequences, antifungal/fungal resistance coding sequences, antiviral/viral resistance coding sequences (including both RNA and DNA viruses), abiotic and biotic stress tolerance coding sequences, or sequences modifying plant traits such as yield, grain quality, nutrient content, starch quality and quantity, nitrogen fixation and/or utilization, fatty acids, and oil content and/or composition. Other polynucleotides of interest include sterility and/or fertility genes, such as for example and not limitation, male sterility and male fertility genes. More specific polynucleotides of interest include, but are not limited to, genes that improve crop yield, genes that decrease crop yield, polynucleotides that improve desirability of crops, genes encoding proteins conferring resistance to abiotic stress, such as drought, nitrogen, temperature, salinity, toxic metals or trace elements, or those conferring resistance to toxins such as pesticides and herbicides, or to biotic stress, such as attacks by fungi, viruses, bacteria, insects, and nematodes, and development of diseases associated with these organisms, and genes conferring herbicide tolerance. General categories of genes of interest include, for example, those genes involved in information, such as zinc fingers, those involved in communication, such as kinases, and those involved in housekeeping, such as heat shock proteins. More specific categories of transgenes, for example, include genes encoding important traits for agronomics, insect resistance, disease resistance, herbicide resistance, fertility or sterility, grain characteristics, and commercial products. Genes of interest include, generally, those involved in oil, starch, carbohydrate, or nutrient metabolism as well as those affecting kernel size, sucrose loading, and the like that can be stacked or used in combination with other traits, such as but not limited to herbicide resistance, described herein. The polypeptide encoded by any of the foregoing polynucleotides may also be used in the methods and compositions herein, such as for example and not limitation, incorporation into a host cell (e.g., a plant cell or protoplast), in a fusion with Argonaute and/or in an expression cassette with Argonaute. One or more polypeptides may be present in said method or composition.

Agronomically important traits such as oil, saccharose, starch, and protein content can be genetically altered in addition to using traditional breeding methods. Modifications include increasing content of oleic acid, saturated and unsaturated oils, increasing levels of lysine and sulfur, providing essential amino acids, and also modification of starch. Hordothionin protein modifications are described in U.S. Pat. Nos. 5,703,049, 5,885,801, 5,885,802, and 5,990,389, herein incorporated by reference. Another example is lysine and/or sulfur rich seed protein encoded by the soybean 2S albumin described in U.S. Pat. No. 5,850,016, and the chymotrypsin inhibitor from barley, described in Williamson et al. (1987) Eur. J. Biochem. 165:99-106, the disclosures of which are herein incorporated by reference.

Commercial traits can also be encoded on a polynucleotide of interest that could increase for example, starch or saccharose for ethanol production, or provide expression of proteins. Another important commercial use of transformed plants is the production of polymers and bioplastics such as described in U.S. Pat. No. 5,602,321. Genes such as β-Ketothiolase, PHBase (polyhydroxybutyrate synthase), and acetoacetyl-CoA reductase (see Schubert et al. (1988) J. Bacteriol. 170:5837-5847) facilitate expression of polyhydroxyalkanoates (PHAs).

Derivatives of the coding sequences can be made by site-directed mutagenesis to increase the level of preselected amino acids in the encoded polypeptide. For example, the gene encoding the barley high lysine polypeptide (BHL) is derived from barley chymotrypsin inhibitor, U.S. application Ser. No. 08/740,682, filed Nov. 1, 1996, and WO 98/20133, the disclosures of which are herein incorporated by reference. Other proteins include methionine-rich plant proteins such as from sunflower seed (Lilley et al. (1989) Proceedings of the World Congress on Vegetable Protein Utilization in Human Foods and Animal Feedstuffs, ed. Applewhite (American Oil Chemists Society, Champaign, Ill.), pp. 497-502; herein incorporated by reference); corn (Pedersen et al. (1986) J. Biol. Chem. 261:6279; Kirihara et al. (1988) Gene 71:359; both of which are herein incorporated by reference); and rice (Musumura et al. (1989) Plant Mol. Biol. 12:123, herein incorporated by reference). Other agronomically important genes encode latex, Floury 2, growth factors, seed storage factors, and transcription factors.

Polynucleotides that improve crop yield include dwarfing genes, such as Rht1 and Rht2 (Peng et al. (1999) Nature 400:256-261), and those that increase plant growth, such as ammonium-inducible glutamate dehydrogenase. Polynucleotides that improve desirability of crops include, for example, those that allow plants to have reduced saturated fat content, those that boost the nutritional value of plants, and those that increase grain protein. Polynucleotides that improve salt tolerance are those that increase or allow plant growth in an environment of higher salinity than the native environment of the plant into which the salt-tolerant gene(s) has been introduced.

Polynucleotides/polypeptides that influence amino acid biosynthesis include, for example, anthranilate synthase (AS; EC 4.1 0.3.27) which catalyzes the first reaction branching from the aromatic amino acid pathway to the biosynthesis of tryptophan in plants, fungi, and bacteria. In plants, the chemical processes for the biosynthesis of tryptophan are compartmentalized in the chloroplast. See, for example, US Pub. 2008/0050506, herein incorporated by reference. Additional sequences of interest include Chorismate Pyruvate Lyase (CPL) which refers to a gene encoding an enzyme which catalyzes the conversion of chorismate to pyruvate and pHBA. The most well characterized CPL gene has been isolated from E. coli and bears the GenBank accession number M96268. See, U.S. Pat. No. 7,361,811, herein incorporated by reference.

Polynucleotide sequences of interest may encode proteins involved in providing disease or pest resistance. By “disease resistance” or “pest resistance” is intended that the plants avoid the harmful symptoms that are the outcome of the plant-pathogen interactions. Pest resistance genes may encode resistance to pests that have great yield drag such as rootworm, cutworm, European Corn Borer, and the like. Disease resistance and insect resistance genes such as lysozymes or cecropins for antibacterial protection, or proteins such as defensins, glucanases or chitinases for antifungal protection, or Bacillus thuringiensis endotoxins, protease inhibitors, collagenases, lectins, or glycosidases for controlling nematodes or insects are all examples of useful gene products. Genes encoding disease resistance traits include detoxification genes, such as against fumonisin (U.S. Pat. No. 5,792,931); avirulence (avr) and disease resistance (R) genes (Jones et al. (1994) Science 266:789; Martin et al. (1993) Science 262:1432; and Mindrinos et al. (1994) Cell 78:1089); and the like. Insect resistance genes may encode resistance to pests that have great yield drag such as rootworm, cutworm, European Corn Borer, and the like. Such genes include, for example, Bacillus thuringiensis toxic protein genes (U.S. Pat. Nos. 5,366,892; 5,747,450; 5,736,514; 5,723,756; 5,593,881; and Geiser et al. (1986) Gene 48:109); and the like.

An “herbicide resistance protein” or a protein resulting from expression of an “herbicide resistance-encoding nucleic acid molecule” includes proteins that confer upon a cell the ability to tolerate a higher concentration of an herbicide than cells that do not express the protein, or to tolerate a certain concentration of an herbicide for a longer period of time than cells that do not express the protein. Herbicide resistance traits may be introduced into plants by genes coding for resistance to herbicides that act to inhibit the action of acetolactate synthase (ALS), in particular the sulfonyl urea-type herbicides, genes coding for resistance to herbicides that act to inhibit the action of glutamine synthase, such as phosphinothricin or basta (e.g., the bar gene), glyphosate (e.g., the EPSP synthase gene and the GAT gene), HPPD inhibitors (e.g, the HPPD gene) or other such genes known in the art. See, for example, U.S. Pat. Nos. 7,626,077, 5,310,667, 5,866,775, 6,225,114, 6,248,876, 7,169,970, 6,867,293, and U.S. Provisional Application No. 61/401,456, each of which is herein incorporated by reference. The bar gene encodes resistance to the herbicide basta, the nptII gene encodes resistance to the antibiotics kanamycin and geneticin, and the ALS-gene mutants encode resistance to the herbicide chlorsulfuron.

Sterility genes can also be encoded in an expression cassette and provide an alternative to physical detasseling, particularly of maize. Examples of genes used in such ways include male fertility genes such as MS26 (see for example U.S. Pat. Nos. 7,098,388, 7,517,975, 7,612,251), MS45 (see for example U.S. Pat. Nos. 5,478,369, 6,265,640) or MSCA1 (see for example U.S. Pat. No. 7,919,676). Other genes include kinases and those encoding compounds toxic to either male or female gametophytic development.

Furthermore, it is recognized that the polynucleotide of interest may also comprise antisense sequences complementary to at least a portion of the messenger RNA (mRNA) for a targeted gene sequence of interest. Antisense nucleotides are constructed to hybridize with the corresponding mRNA.

Modifications of the antisense sequences may be made as long as the sequences hybridize to and interfere with expression of the corresponding mRNA. In this manner, antisense constructions having 70%, 80%, or 85% sequence identity to the corresponding antisense sequences may be used. Furthermore, portions of the antisense nucleotides may be used to disrupt the expression of the target gene. Generally, sequences of at least 50 nucleotides, 100 nucleotides, 200 nucleotides, or greater may be used.

In addition, the polynucleotide of interest may also be used in the sense orientation to suppress the expression of endogenous genes in plants. Methods for suppressing gene expression in plants using polynucleotides in the sense orientation are known in the art. The methods generally involve transforming plants with a DNA construct comprising a promoter that drives expression in a plant operably linked to at least a portion of a nucleotide sequence that corresponds to the transcript of the endogenous gene. Typically, such a nucleotide sequence has substantial sequence identity to the sequence of the transcript of the endogenous gene, generally greater than about 65% sequence identity, about 85% sequence identity, or greater than about 95% sequence identity. See, U.S. Pat. Nos. 5,283,184 and 5,034,323; herein incorporated by reference.

The polynucleotide of interest can also be a phenotypic marker. A phenotypic marker is screenable or a selectable marker that includes visual markers and selectable markers whether it is a positive or negative selectable marker. Any phenotypic marker can be used. Specifically, a selectable or screenable marker comprises a DNA segment that allows one to identify, or select for or against a molecule or a cell that contains it, often under particular conditions. These markers can encode an activity, such as, but not limited to, production of RNA, peptide, or protein, or can provide a binding site for RNA, peptides, proteins, inorganic and organic compounds or compositions and the like.

Examples of selectable markers include, but are not limited to, DNA segments that comprise restriction enzyme sites; DNA segments that encode products which provide resistance against otherwise toxic compounds including antibiotics, such as, spectinomycin, ampicillin, kanamycin, tetracycline, Basta, neomycin phosphotransferase II (NEO) and hygromycin phosphotransferase (HPT)); DNA segments that encode products which are otherwise lacking in the recipient cell (e.g., tRNA genes, auxotrophic markers); DNA segments that encode products which can be readily identified (e.g., phenotypic markers such as β-galactosidase, GUS; fluorescent proteins such as green fluorescent protein (GFP), cyan (CFP), yellow (YFP), red (RFP), yellow-green fluorescent protein (mNeonGreen) and cell surface proteins); the generation of new primer sites for PCR (e.g., the juxtaposition of two DNA sequence not previously juxtaposed), the inclusion of DNA sequences not acted upon or acted upon by a restriction endonuclease or other DNA modifying enzyme, chemical, etc.; and, the inclusion of a DNA sequences required for a specific modification (e.g., methylation) that allows its identification. Additional selectable markers include genes that confer resistance to herbicidal compounds, such as glufosinate ammonium, bromoxynil, imidazolinones, and 2,4-dichlorophenoxyacetate (2,4-D). See for example, Yarranton, (1992) Curr Opin Biotech 3:506-11; Christopherson et al., (1992) Proc. Natl. Acad. Sci. USA 89:6314-8; Yao et al., (1992) Cell 71:63-72; Reznikoff, (1992) Mol Microbiol 6:2419-22; Hu et al., (1987) Cell 48:555-66; Brown et al., (1987) Cell 49:603-12; Figge et al., (1988) Cell 52:713-22; Deuschle et al., (1989) Proc. Natl. Acad. Sci. USA 86:5400-4; Fuerst et al., (1989) Proc. Natl. Acad. Sci. USA 86:2549-53; Deuschle et al., (1990) Science 248:480-3; Gossen, (1993) Ph.D. Thesis, University of Heidelberg; Reines et al., (1993) Proc. Natl. Acad. Sci. USA 90:1917-21; Labow et al., (1990) Mol Cell Biol 10:3343-56; Zambretti et al., (1992) Proc. Natl. Acad. Sci. USA 89:3952-6; Bairn et al., (1991) Proc. Natl. Acad. Sci. USA 88:5072-6; Wyborski et al., (1991) Nucleic Acids Res 19:4647-53; Hillen and Wissman, (1989) Topics Mol Struc Biol 10:143-62; Degenkolb et al., (1991) Antimicrob Agents Chemother 35:1591-5; Kleinschnidt et al., (1988) Biochemistry 27:1094-104; Bonin, (1993) Ph.D. Thesis, University of Heidelberg; Gossen et al., (1992) Proc. Natl. Acad. Sci. USA 89:5547-51; Oliva et al., (1992) Antimicrob Agents Chemother 36:913-9; Hlavka et al., (1985) Handbook of Experimental Pharmacology, Vol. 78 (Springer-Verlag, Berlin); Gill et al., (1988) Nature 334:721-4.

Exogenous products include plant enzymes and products as well as those from other sources including procaryotes and other eukaryotes. Such products include enzymes, cofactors, hormones, and the like. The level of proteins, particularly modified proteins having improved amino acid distribution to improve the nutrient value of the plant, can be increased. This is achieved by the expression of such proteins having enhanced amino acid content. The transgenes, recombinant DNA molecules, DNA sequences of interest, and polynucleotides of interest can be comprise one or more DNA sequences for gene silencing. Methods for gene silencing involving the expression of DNA sequences in plant are known in the art include, but are not limited to, cosuppression, antisense suppression, double-stranded RNA (dsRNA) interference, hairpin RNA (hpRNA) interference, intron-containing hairpin RNA (ihpRNA) interference, transcriptional gene silencing, and micro RNA (miRNA) interference.

In some embodiments, the nucleic acid must be optimized for expression in plants. As used herein, a “plant-optimized nucleotide sequence” is a nucleotide sequence that has been optimized for increased expression in plants, particularly for increased expression in plants or in one or more plants of interest. For example, a plant-optimized nucleotide sequence can be synthesized by modifying a nucleotide sequence encoding a protein such as, for example, double-strand-break-inducing agent (e.g., an endonuclease) as disclosed herein, using one or more plant-preferred codons for improved expression. See, for example, Campbell and Gowri (1990) Plant Physiol. 92:1-11 for a discussion of host-preferred codon usage.

Methods are available in the art for synthesizing plant-preferred genes. See, for example, U.S. Pat. Nos. 5,380,831, and 5,436,391, and Murray et al. (1989) Nucleic Acids Res. 17:477-498, herein incorporated by reference. Additional sequence modifications are known to enhance gene expression in a plant host. These include, for example, elimination of: one or more sequences encoding spurious polyadenylation signals, one or more exon-intron splice site signals, one or more transposon-like repeats, and other such well-characterized sequences that may be deleterious to gene expression. The G-C content of the sequence may be adjusted to levels average for a given plant host, as calculated by reference to known genes expressed in the host plant cell. When possible, the sequence is modified to avoid one or more predicted hairpin secondary mRNA structures. Thus, “a plant-optimized nucleotide sequence” of the present disclosure comprises one or more of such sequence modifications.

Transformation Methods for Use with the Invention

A variety of methods are known for the introduction of nucleotide sequences and polypeptides into an organism, including, for example, transformation, sexual crossing, and the introduction of the polypeptide, DNA, or mRNA into the cell.

In some embodiments, the invention comprises breeding of plants comprising one or more transgenic traits. Most commonly, transgenic traits are randomly inserted throughout the plant genome as a consequence of bacterial transformation systems, such as for example and not limitation, those based on Agrobacterium, biolistics, or other commonly used procedures. More recently, gene targeting protocols have been developed that enable directed transgene insertion. One important technology, site-specific integration (SSI) enables the targeting of a transgene to the same chromosomal location as a previously inserted transgene. Custom-designed meganucleases and custom-designed zinc finger meganucleases allow researchers to design nucleases to target specific chromosomal locations, and these reagents allow the targeting of transgenes at the chromosomal site cleaved by these nucleases.

The currently used systems for precision genetic engineering of eukaryotic genomes, e.g., plant genomes, rely upon homing endonucleases, meganucleases, zinc finger nucleases, and transcription activator-like effector nucleases (TALENs), which require de novo protein engineering for every new target locus. The highly specific, DNA-directed DNA nuclease Argonaute endonuclease system described herein, is more easily customizable and therefore more useful when modification of many different target sequences is the goal.

Transformation methods in plants may include direct and indirect methods of transformation and are applicable for dicotyledonous and mostly for monocots. Delivery into plant cells by any of the above methods may further include use of one or more cell-penetrating peptides (CPPs). Cells suitable for transformation include, for example and not limitation, plastids and protoplasts.

Suitable direct transformation methods include, for example and not limitation, PEG-induced DNA uptake, pollen tube mediated introduction directly into fertilized embryos/zygotes, liposome-mediated transformation, biolistic methods, by means of particle bombardment, electroporation or microinjection. Indirect methods include, for example and not limitation, bacteria-mediated transformation, (e.g., the Agrobacterium-mediated transformation technology) or viral infection using viral vectors.

Methods for contacting, providing, and/or introducing a composition into various organisms are known and include but are not limited to, stable transformation methods, transient transformation methods, virus-mediated methods, and sexual breeding. Stable transformation indicates that the introduced polynucleotide integrates into the genome of the organism and is capable of being inherited by progeny thereof. Transient transformation indicates that the introduced composition is only temporarily expressed or present in the organism. Protocols for introducing polynucleotides and polypeptides into plants may vary depending on the type of plant or plant cell targeted for transformation, such as monocot or dicot. Suitable methods of introducing polynucleotides and polypeptides into plant cells and subsequent insertion into the plant genome include (in addition to those listed herein) polyethylene glycol-mediated transformation, microparticle bombardment, pollen-tube mediated introduction into fertilized embryos/zygotes, microinjection (Crossway et al., (1986) Biotechniques 4:320-34 and U.S. Pat. No. 6,300,543), meristem transformation (U.S. Pat. No. 5,736,369), electroporation (Riggs et al., (1986) Proc. Natl. Acad. Sci. USA 83:5602-6), Agrobacterium-mediated transformation (U.S. Pat. Nos. 5,563,055 and 5,981,840), direct gene transfer (Paszkowski et al., (1984) EMBO J 3:2717-22), and ballistic particle acceleration (U.S. Pat. Nos. 4,945,050; 5,879,918; 5,886,244; 5,932,782; Tomes et al., (1995) “Direct DNA Transfer into Intact Plant Cells via Microprojectile Bombardment” in Plant Cell, Tissue, and Organ Culture: Fundamental Methods, ed. Gamborg & Phillips (Springer-Verlag, Berlin); McCabe et al., (1988) Biotechnology 6:923-6; Weissinger et al., (1988) Ann Rev Genet 22:421-77; Sanford et al., (1987) Particulate Science and Technology 5:27-37 (onion); Christou et al., (1988) Plant Physiol 87:67-74 (soybean); Finer and McMullen, (1991) In Vitro Cell Dev Biol 27P:175-82 (soybean); Singh et al., (1998) Theor Appl Genet 96:319-24 (soybean); Datta et al., (1990) Biotechnology 8:736-40 (rice); Klein et al., (1988) Proc. Natl. Acad. Sci. USA 85:4305-9 (maize); Klein et al., (1988) Biotechnology 6:559-63 (maize); U.S. Pat. Nos. 5,240,855; 5,322,783 and 5,324,646; Klein et al., (1988) Plant Physiol 91:440-4 (maize); Fromm et al., (1990) Biotechnology 8:833-9 (maize); Hooykaas-Van Slogteren et al., (1984) Nature 311:763-4; U.S. Pat. No. 5,736,369 (cereals); Bytebier et al., (1987) Proc. Natl. Acad. Sci. USA 84:5345-9 (Liliaceae); De Wet et al., (1985) in The Experimental Manipulation of Ovule Tissues, ed. Chapman et al., (Longman, N.Y.), pp. 197-209 (pollen); Kaeppler et al., (1990) Plant Cell Rep 9:415-8) and Kaeppler et al., (1992) Theor Appl Genet 84:560-6 (whisker-mediated transformation); D'Halluin et al., (1992) Plant Cell 4:1495-505 (electroporation); Li et al., (1993) Plant Cell Rep 12:250-5; Christou and Ford (1995) Annals Botany 75:407-13 (rice) and Osjoda et al., (1996) Nat Biotechnol 14:745-50 (maize via Agrobacterium tumefaciens).

Alternatively, polynucleotides may be introduced into plants by contacting plants with a virus or viral nucleic acids. Generally, such methods involve incorporating a polynucleotide within a viral DNA or RNA molecule. In some examples a polypeptide of interest may be initially synthesized as part of a viral polyprotein, which is later processed by proteolysis in vivo or in vitro to produce the desired recombinant protein. Methods for introducing polynucleotides into plants and expressing a protein encoded therein, involving viral DNA or RNA molecules, are known, see, for example, U.S. Pat. Nos. 5,889,191, 5,889,190, 5,866,785, 5,589,367 and 5,316,931. Transient transformation methods include, but are not limited to, the introduction of polypeptides, such as a double-strand break inducing agent, directly into the organism, the introduction of polynucleotides such as DNA and/or RNA polynucleotides, and the introduction of the RNA transcript, such as an mRNA encoding a double-strand break inducing agent, into the organism. Such methods include, for example, microinjection or particle bombardment. See, for example Crossway et al, (1986) Mol Gen Genet 202:179-85; Nomura et al, (1986) Plant Sci 44:53-8; Hepler et al., (1994) Proc. Natl. Acad. Sci. USA 91:2176-80; and Hush et al., (1994) J Cell Sci 107:775-84.

### Genetic Constructs of the Invention

The present disclosure further provides expression constructs, such as for example and not limitation an expression cassette, for expressing in a host (e.g., a plant, plant cell, or plant part) an Argonaute system that is capable of binding to and creating a double strand break in a target site. In one embodiment, the expression constructs of the disclosure comprise a promoter operably linked to a nucleotide sequence encoding an Argonaute gene and a promoter operably linked to a guide nucleic acid of the present disclosure. The promoter is capable of driving expression of an operably linked nucleotide sequence in a host (e.g., a plant) cell. In another embodiment, the Argonaute gene comprises one or more transcriptional and/or translational fusions as described herein. In some embodiments, the expression cassette allows transient expression of the Argonaute system, while in other embodiments, the expression cassette allows the Argonaute system to be stably maintained within the host cell, such as for example and not limitation, by integration into the host cell genome.

A promoter is a region of DNA involved in recognition and binding of RNA polymerase and other proteins to initiate transcription. Promoters are well known in the art to be highly specific and adapted for use in particular kingdoms, genera, species, and even particular tissues within the same organism. Promoters can be constitutively active or inducible; examples of each are well known in the art. For example, a plant promoter is a promoter capable of initiating transcription in a plant cell, for a review of plant promoters, see, Potenza et al, (2004) In Vitro Cell Dev Biol 40:1-22. Constitutive promoters include, for example, the core promoter of the Rsyn7 promoter and other constitutive promoters disclosed in WO99/43838 and U.S. Pat. No. 6,072,050; the core CaMV 35S promoter (Odell et al., (1985) Nature 313:810-2); rice actin (McElroy et al., (1990) Plant Cell 2:163-71); ubiquitin (Christensen et al., (1989) Plant Mol Biol 12:619-32; Christensen et al., (1992) Plant Mol Biol 18:675-89); pEMU (Last et al., (1991) Theor Appl Genet 81:581-8); MAS (Velten et al., (1984) EMBO J 3:2723-30); ALS promoter (U.S. Pat. No. 5,659,026), and the like. Other constitutive promoters are described in, for example, U.S. Pat. Nos. 5,608,149; 5,608,144; 5,604,121; 5,569,597; 5,466,785; 5,399,680; 5,268,463; 5,608,142 and 6,177,611.

In some embodiments, an inducible promoter may be used. Pathogen-inducible promoters induced following infection by a pathogen include, but are not limited to those regulating expression of PR proteins, SAR proteins, beta-1,3-glucanase, chitinase, etc.

Chemical-regulated promoters can be used to modulate the expression of a gene in a plant through the application of an exogenous chemical regulator. The promoter may be a chemical-inducible promoter, where application of the chemical induces gene expression, or a chemical-repressible promoter, where application of the chemical represses gene expression. Chemical-inducible promoters include, but are not limited to, the maize ln2-2 promoter, activated by benzene sulfonamide herbicide safeners (De Veylder et al., (1997) Plant Cell Physiol 38:568-77), the maize GST promoter (GST-ll-27, WO93/01294), activated by hydrophobic electrophilic compounds used as pre-emergent herbicides, and the tobacco PR-1 a promoter (Ono et al., (2004) Biosci Biotechnol Biochem 68:803-7) activated by salicylic acid. Other chemical-regulated promoters include steroid-responsive promoters (see, for example, the glucocorticoid-inducible promoter (Schena et al., (1991) Proc. Natl. Acad. Sci. USA 88:10421-5; McNellis et al., (1998) Plant J 14:247-257); tetracycline-inducible and tetracycline-repressible promoters (Gatz et al., (1991) Mol Gen Genet 227:229-37; U.S. Pat. Nos. 5,814,618 and 5,789,156).

Tissue-preferred promoters can be utilized to target enhanced expression within a particular plant tissue. Tissue-preferred promoters include, for example, Kawamata et al., (1997) Plant Cell Physiol 38:792-803; Hansen et al., (1997) Mol Gen Genet 254:337-43; Russell et al., (1997) Transgenic Res 6:157-68; Rinehart et al., (1996) Plant Physiol 1 12:1331-41; Van Camp et al., (1996) Plant Physiol 112:525-35; Canevascini et al., (1996) Plant Physiol 112:513-524; Lam, (1994) Results Probl Cell Differ 20:181-96; and Guevara-Garcia et al., (1993) Plant J 4:495-505. Leaf-preferred promoters include, for example, Yamamoto et al., (1997) Plant J 12:255-65; Kwon et al., (1994) Plant Physiol 105:357-67; Yamamoto et al., (1994) Plant Cell Physiol 35:773-8; Gotor et al., (1993) Plant J 3:509-18; Orozco et al., (1993) Plant Mol Biol 23:1 129-38; Matsuoka et al., (1993) Proc. Natl. Acad. Sci. USA 90:9586-90; Simpson et al., (1958) EMBO J 4:2723-9; Timko et al., (1988) Nature 318:57-8. Root-preferred promoters include, for example, Hire et al., (1992) Plant Mol Biol 20:207-18 (soybean root-specific glutamine synthase gene); Miao et al., (1991) Plant Cell 3:11-22 (cytosolic glutamine synthase (GS)); Keller and Baumgartner, (1991) Plant Cell 3:1051-61 (root-specific control element in the GRP 1 0.8 gene of French bean); Sanger et al., (1990) Plant Mol Biol 14:433-43 (root-specific promoter of A. tumefaciens mannopine synthase (MAS)); Bogusz et al., (1990) Plant Cell 2:633-41 (root-specific promoters isolated from Parasponia andersonii and Trema tomentosa); Leach and Aoyagi, (1991) Plant Sci 79:69-76 (A. rhizogenes rolC and rolD root-inducing genes); Teeri et al., (1989) EMBO J 8:343-50 (Agrobacterium wound-induced TR1′ and TR2′ genes); VfENOD-GRP3 gene promoter (Kuster et al., (1995) Plant Mol Biol 29:759-72); and rolB promoter (Capana et al., (1994) Plant Mol Biol 25:681-91; phaseolin gene (Murai et al., (1983) Science 23:476-82; Sengopta-Gopalen et al., (1988) Proc. Natl. Acad. Sci. USA 82:3320-4). See also, U.S. Pat. Nos. 5,837,876; 5,750,386; 5,633,363; 5,459,252; 5,401,836; 5,110,732 and 5,023,179.

Seed-preferred promoters include both seed-specific promoters active during seed development, as well as seed-germinating promoters active during seed germination. See, Thompson et al., (1989) BioEssays 10:108. Seed-preferred promoters include, but are not limited to, Cim1 (cytokinin-induced message); cZ19B1 (maize 19 kDa zein); and milps (myo-inositol-1-phosphate synthase); (WO00/11177; and U.S. Pat. No. 6,225,529). For dicots, seed-preferred promoters include, but are not limited to, bean β-phaseolin, napin, β-conglycinin, soybean lectin, cruciferin, and the like. For monocots, seed-preferred promoters include, but are not limited to, maize 15 kDa zein, 22 kDa zein, 27 kDa gamma zein, waxy, shrunken 1, shrunken 2, globulin 1, oleosin, and nud. See also, WO00/12733, where seed-preferred promoters from END1 and END2 genes are disclosed.

A phenotypic marker is a screenable or selectable marker that includes visual markers and selectable markers whether it is a positive or negative selectable marker. Any phenotypic marker can be used. Specifically, a selectable or screenable marker comprises a DNA segment that allows one to identify, or select for or against a molecule or a cell that contains it, often under particular conditions. These markers can encode an activity, such as, but not limited to, production of RNA, peptide, or protein, or can provide a binding site for RNA, peptides, proteins, inorganic and organic compounds or compositions and the like.

Examples of selectable markers include, but are not limited to, DNA segments that comprise restriction enzyme sites; DNA segments that encode products which provide resistance against otherwise toxic compounds including antibiotics, such as, spectinomycin, ampicillin, kanamycin, tetracycline, Basta, neomycin phosphotransferase II (NEO) and hygromycin phosphotransferase (HPT)); DNA segments that encode products which are otherwise lacking in the recipient cell (e.g., tRNA genes, auxotrophic markers); DNA segments that encode products which can be readily identified (e.g., phenotypic markers such as β-galactosidase, GUS; fluorescent proteins such as green fluorescent protein (GFP), cyan (CFP), yellow (YFP), yellow-green (mNeonGreen), red (RFP), and cell surface proteins); the generation of new primer sites for PCR (e.g., the juxtaposition of two DNA sequence not previously juxtaposed), the inclusion of DNA sequences not acted upon or acted upon by a restriction endonuclease or other DNA modifying enzyme, chemical, etc.; and, the inclusion of a DNA sequences required for a specific modification (e.g., methylation) that allows its identification.

Additional selectable markers include genes that confer resistance to herbicidal compounds, such as glufosinate ammonium, bromoxynil, imidazolinones, and 2,4-dichlorophenoxyacetate (2,4-D). See for example, Yarranton, (1992) Curr Opin Biotech 3:506-1 1; Christopherson et al., (1992) Proc. Natl. Acad. Sci. USA 89:6314-8; Yao et al., (1992) Cell 71:63-72; Reznikoff, (1992) Mol Microbiol 6:2419-22; Hu et al., (1987) Cell 48:555-66; Brown et al., (1987) Cell 49:603-12; Figge et al., (1988) Cell 52:713-22; Deuschle et al., (1989) Proc. Natl. Acad. Sci. USA 86:5400-4; Fuerst et al., (1989) Proc. Natl. Acad. Sci. USA 86:2549-53; Deuschle et al., (1990) Science 248:480-3; Gossen, (1993) Ph.D. Thesis, University of Heidelberg; Reines et al., (1993) Proc. Natl. Acad. Sci. USA 90:1917-21; Labow et al., (1990) Mol Cell Biol 10:3343-56; Zambretti et al., (1992) Proc. Natl. Acad. Sci. USA 89:3952-6; Bairn et al., (1991) Proc. Natl. Acad. Sci. USA 88:5072-6; Wyborski et al., (1991) Nucleic Acids Res 19:4647-53; Hillen and Wissman, (1989) Topics Mol Struc Biol 10:143-62; Degenkolb et al., (1991) Antimicrob Agents Chemother 35:1591-5; Kleinschnidt et al., (1988) Biochemistry 27:1094-104; Bonin, (1993) Ph.D. Thesis, University of Heidelberg; Gossen et al., (1992) Proc. Natl. Acad. Sci. USA 89:5547-51; Oliva et al., (1992) Antimicrob Agents Chemother 36:913-9; Hlavka et al, (1985) Handbook of Experimental Pharmacology, Vol. 78 (Springer-Verlag, Berlin); Gill et al, (1988) Nature 334:721-4.

### Transgenic Plants, Plant Parts, Cells and Seeds of the Invention

In a preferred embodiment of the invention, transgenic plants including transgenic parts of the transgenic plant, in particular transgenic seeds and transgenic cells are provided. The transgenic parts of the transgenic plant can further include those parts which can be harvested, such as for example and not limitation, the beets for sugar beet, rice grains for rice, and corn cobs for maize.

For production of transgenic seeds carrying the integrated nucleic acid construct, the transgenic plant may be selfed. Alternatively, the transgenic plant can be crossed with a similar transgenic plant or with a transgenic plant which carries one or more nucleic acids that are different from the invented genetic constructs, or with a non-transgenic plant of known plant breeding methods to produce transgenic seeds. These seeds can be used to provide progeny generations of transgenic plants of the invention, comprising the integrated nucleic acid from the invented genetic constructs.

Suitable methods of transforming plant cells are known in plant biotechnology and are described herein. Each of these methods can be used to preferentially introduce a selected nucleic acid into a vector into a plant cell to obtain a transgenic plant of the present invention. Transformation methods may include direct and indirect methods of transformation and are applicable for dicotyledonous and mostly for monocots.

Transformed plant cells, including protoplasts and plastids, are selected for one or more markers which have been transformed with the nucleic acid of the invention into the plant and include genes that mediate preferably antibiotic resistance, such as the neomycin phosphotransferase II-mediated gene NPTII, which encodes kanamycin resistance.

Subsequently, the transformed cells are regenerated into whole plants. Following DNA transfer and regeneration, the plants can be checked for example the quantitative PCR for the presence of the nucleic acid of the invention.

The cells having the introduced sequence may be grown or regenerated into plants using conventional conditions, see for example, McCormick et al, (1986) Plant Cell Rep 5:81-4. These plants may then be grown, and either pollinated with the same transformed strain or with a different transformed or untransformed strain, and the resulting progeny having the desired characteristic and/or comprising the introduced polynucleotide or polypeptide identified. Two or more generations may be grown to ensure that the polynucleotide is stably maintained and inherited, and seeds harvested.

Any plant can be used, including monocot and dicot plants. Examples of monocot plants that can be used include, but are not limited to, corn (Zea mays), rice (Oryza sativa), rye (Secale cereale), sorghum (Sorghum bicolor, Sorghum vulgare), millet (e.g., pearl millet (Pennisetum glaucum), proso millet (Panicum miliaceum), foxtail millet (Setaria italica), finger millet (Eleusine coracana)), wheat (Triticum aestivum), sugarcane (Saccharum spp.), oats (Avena), barley (Hordeum), switchgrass (Panicum virgatum), pineapple (Ananas comosus), banana (Musa spp.), palm, ornamentals, turfgrasses, and other grasses. Examples of dicot plants that can be used include, but are not limited to, soybean (Glycine max), canola (Brassica napus and B. campestris), alfalfa (Medicago sativa), tobacco (Nicotiana tabacum), Arabidopsis (Arabidopsis thaliana), sunflower (Helianthus annuus), sugar beet (Beta vulgaris), cotton (Gossypium arboreum), and peanut (Arachis hypogaea), tomato (Solanum lycopersicum), potato (Solanum tuberosum) etc.

Additional non-limiting exemplary plants for use with the invented methods and compositions include Hordeum vulgare, Hordeum bulbusom, Sorghum bicolor, Saccharum officinarium, Zea mays, Setaria italica, Oryza minuta, Oriza sativa, Oryza australiensis, Oryza alta, Triticum aestivum, Triticum durum, Secale cereale, Triticale, Malus domestica, Brachypodium distachyon, Hordeum marinum, Aegilops tauschii, Daucus glochidiatus, Beta vulgaris, Daucus pusillus, Daucus muricatus, Daucus carota, Eucalyptus grandis, Nicotiana sylvestris, Nicotiana tomentosiformis, Nicotiana tabacum, Nicotiana benthamiana, Solanum lycopersicum, Solanum tuberosum, Coffea canephora, Vitis vinifera, Erythrante guttata, Genlisea aurea, Cucumis sativus, Morus notabilis, Arabidopsis arenosa, Arabidopsis lyrata, Arabidopsis thaliana, Crucihimalaya himalaica, Crucihimalaya wallichii, Cardamine flexuosa, Lepidium virginicum, Capsella bursa pastoris, Olmarabidopsis pumila, Arabis hirsute, Brassica napus, Brassica oleracea, Brassica rapa, Raphanus sativus, Brassica juncacea, Brassica nigra, Eruca vesicaria subsp. sativa, Citrus sinensis, Jatropha curcas, Populus trichocarpa, Medicago truncatula, Cicer yamashitae, Cicer bijugum, Cicer arietinum, Cicer reticulatum, Cicer judaicum, Cajanus cajanifolius, Cajanus scarabaeoides, Phaseolus vulgaris, Glycine max, Gossypium sp., Astragalus sinicus, Lotus japonicas, Torenia fournieri, Allium cepa, Allium fistulosum, Allium sativum, Helianthus annuus, Helianthus tuberosus and Allium tuberosum, or any variety or subspecies belonging to one of the aforementioned plants.

Treatment Methods for Use with the Invention

The invented method provides a method for treating diseases and/or conditions (such as for example and not limitation, diseases caused by insect(s)). The invented method further provides a method for preventing insect infection and/or infestation in a plant (e.g., insect resistance).

Non-limiting examples of the diseases and/or conditions treatable by the invented methods include Anthracnose Stalk Rot, Aspergillus Ear Rot, Common Corn Ear Rots, Corn Ear Rots (Uncommon), Common Rust of Corn, Diplodia Ear Rot, Diplodia Leaf Streak, Diplodia Stalk Rot, Downy Mildew, Eyespot, Fusarium Ear Rot, Fusarium Stalk Rot, Gibberella Ear Rot, Gibberella Stalk Rot, Goss's Wilt and Leaf Blight, Gray Leaf Spot, Head Smut, Northern Corn Leaf Blight, Physoderma Brown Spot, Pythium, Southern Leaf Blight, Southern Rust, and Stewart's Bacterial Wilt and Blight, and combinations thereof.

Non-limiting examples of the insects causing, directly or indirectly, diseases and/or conditions treatable by the invented methods include Armyworm, Asiatic Garden Beetle, Black Cutworm, Brown Marmorated Stink Bug, Brown Stink Bug, Common Stalk Borer, Corn Billbugs, Corn Earworm, Corn Leaf Aphid, Corn Rootworm, Corn Rootworm Silk Feeding, European Corn Borer, Fall Armyworm, Grape Colaspis, Hop Vine Borer, Japanese Beetle, Scouting for Fall Armyworm, Seedcorn Beetle, Seedcorn Maggot, Southern Corn Leaf Beetle, Southwestern Corn Borer, Spider Mite, Sugarcane Beetle, Western Bean Cutworm, White Grub, and Wireworms, and combinations thereof. The invented methods are also suitable for preventing infections and/or infestations of a plant by any such insect(s).

Additional methods and compositions for use with the present invention are found in US2015/089681.

### EXAMPLES

The present invention is also described and demonstrated by way of the following examples. However, the use of these and other examples anywhere in the specification is illustrative only and in no way limits the scope and meaning of the invention or of any exemplified term. Likewise, the invention is not limited to any particular preferred embodiments described here. Indeed, many modifications and variations of the invention may be apparent to those skilled in the art upon reading this specification, and such variations can be made without departing from the invention in spirit or in scope. The invention is therefore to be limited only by the terms of the appended claims along with the full scope of equivalents to which those claims are entitled.

### Example 1: Cassettes for Plant-Optimized Expression of NgAgo and for Measuring Endonuclease Activity

To test activity of the NgAgo endonuclease in plant cells, the WT NgAgo protein sequence (GenBank Accession Number AFZ73749) is amended with an N-terminal MASS sequence for optimal translation initiation in plants followed immediately by an SV40 NLS sequence and a C-terminal Nucleopasmin NLS sequence followed immediately by an HA tag for antibody detection (2NLS-NgAgo; SEQ ID NO: 1). To demonstrate the activity of the NgAgo endonuclease in plant cells, this optimized protein is reverse-translated with codon usage for high expression in plants and then is placed in a strong constitutive expression cassette. A similar cassette is designed for expression of a 2NLS-NgAgo endonuclease with a C-terminal translational fusion to the green fluorescent reporter mNeonGreen (2NLS-NgAgo-mNeonGreen; SEQ ID NO: 2). These expression cassettes (SEQ ID NO: 3 & SEQ ID NO: 4) are cloned into a minimal plasmid vector backbone.

A third plasmid is generated as a vector for co-delivery of episomal targets for testing the endonuclease activity. It contains a strong constitutive expression cassette for a tdTomato fluorescent reporter, followed by a cloning site for the endonuclease target followed by a mNeonGreen coding sequence that would be out of frame relative to the tdTomato reporter. Endonuclease cleavage of the target site results in NHEJ repair, and some frequency of those repair events will generate frameshifts that cause expression of the mNeonGreen protein. Relative cleavage efficiency under different conditions, or of different nucleases, or of different guide-DNAs is measured by comparing the populations of cells expressing tdTomato and mNeonGreen relative to the populations of cells expressing tdTomato alone. This type of test construct is commonly referred to as a “traffic light reporter” (TLR) by those skilled in the art.

### Example 2: Proper Subcellular Localization of Expressed 2NLS-NgAgo and Cutting of an Episomal Target

To demonstrate robust expression and proper subcellular localization of the 2NLS-NgAgo plant-optimized gene, a plasmid containing the 2NLS-NgAgo-mNeonGreen expression cassette is transformed into protoplasts isolated from young leaves of corn and Nicotiana benthamiana plants and monitored for subcellular accumulation. A strong nuclear signal of the mNeonGreen reporter indicates robust expression and proper subcellular localization of the endonuclease protein.

To demonstrate activity of NgAgo in monocot and dicot plant cells and at various plant-optimized temperatures, protoplasts are isolated from young leaves of corn and Nicotiana benthamiana plants and transformed with vectors containing the 2NLS-NgAgo expression cassette and the TLR with the endonuclease target. In addition, 5′-phosphorylated, single-stranded DNA of various lengths is cotransformed to serve as guide-DNA for the appropriate target sequences. After transformation, cells are incubated for at least 24 hours at various temperatures between 18° C. and 37° C. Relative nuclease activity is assessed by flow cytometry to compare the population of cells expressing tdTomato and mNeonGreen relative to the population of cells expressing tdTomato alone.

### Example 3: Targeted Mutations of Chromosomal Sites by NgAgo in Protoplasts

To demonstrate the utility of NgAgo for inducing targeted mutations at chromosomal targets, protoplasts are isolated from young leaves of corn plants and transformed with vectors containing the 2NLS-NgAgo or 2NLS-NgAgo-mNeonGreen expression cassettes. In addition, 5′-phosphorylated, single-stranded DNA is cotransformed to serve as guide-DNA for the appropriate target sequences in the corn genome. Targeted mutations are identified by PCR-based assays, by targeted Next Generation Sequencing (NGS; also known as deep sequencing) of the PCR-amplified target, or by loss of signal from an integrated tdTomato fluorescent reporter.

To demonstrate the utility of NgAgo for inducing multiplex editing events at chromosomal targets, the same experiment is repeated with cotransformation of two 5′-phosphorylated, single-stranded guide-DNA molecules. Targeted mutations are identified by PCR-based assays, by targeted NGS of the PCR-amplified target, or by loss of signal from an integrated tdTomato fluorescent reporter.

### Example 4: Targeted Mutagenesis of Chromosomal Sites by NgAgo in Regenerative Tissues Followed by Plant Regeneration and Inheritance of Mutations

To demonstrate the use of NgAgo for generation of heritable gene editing events, a vector containing an herbicide selection marker and a vector containing the 2NLS-NgAgo expression cassette are bombarded into corn callus tissue, together with 5′-phosphorylated, single-stranded DNA to serve as guide-DNA against a chromosomal target. Plantlets are regenerated from the bombarded tissue and screened by phenotypic, PCR-based, and sequencing assays for mutations at the chromosomal target. Plants harboring targeted mutations are selfed and the progeny screened for inheritance of the mutations.

### Example 5: Use of NgAgo for Gene Editing in Protoplasts

To demonstrate the utility of NgAgo for gene editing at chromosomal targets in plant cells, protoplasts are isolated from young leaves of corn plants and transformed with vectors containing the 2NLS-NgAgo expression cassette, a 5′-phosphorylated, single-stranded DNA to serve as guide-DNA for the appropriate chromosomal target sequence, and a DNA repair template for proper repair of the chromosomal target. Gene editing is assessed by flow cytometry to identify the number of cells expressing a fluorescent reporter signal derived from targeted repair by the template. Proper repair is confirmed by PCR amplification and sequencing.

### Example 6: Use of Guide-DNA Containing Modified Bases for Targeted Mutagenesis in Protoplasts with NgAgo

To demonstrate the use of NgAgo in combination with guide-DNAs containing modified bases, protoplasts are isolated from young leaves of corn plants and transformed with vectors containing the 2NLS-NgAgo expression cassette and with or without the TLR with the endonuclease target. In addition, 5′-phosphorylated, single-stranded DNA containing modified bases is cotransformed to serve as guide-DNA for the appropriate target sequences. Relative nuclease activity using guide-DNAs with and without various modifications is assessed by flow cytometry to compare the population of cells expressing tdTomato and mNeonGreen relative to the population of cells expressing tdTomato alone. Nuclease activity at chromosomal targets is assessed by PCR-based assays, by targeted NGS of the PCR-amplified target, or by loss of signal from an integrated tdTomato fluorescent reporter

### SEQUENCE LISTING

>SEQ ID NO: 1 (2NLS-NgAgo: WT NgAgo amended with N- and C-terminal sequences for optimal translation, nuclear localization, and antibody detection)

MASSPKKKRKVMTVIDLDSTTTADELTSGHTYDISVTLTGVYDNTDEQHP
RMSLAFEQDNGERRYITLWKNTTPKDVETYDYATGSTYIFTNIDYEVKDG
YENLTATYQTTVENATAQEVGTTDEDETFAGGEPLDHEILDDALNETPDD
AETESDSGHVMTSFASRDQLPEWTLHTYTLTATDGAKTDTEYARRTLAYT
VRQELYTDHDAAPVATDGLMLLTPEPLGETPLDLDCGVRVEADETRTLDY
TTAKDRLLARELVEEGLKRSLWDDYLVRGIDEVLSKEPVLTCDEFDLHER
YDLSVEVGHSGRAYLHINFRHREVPKLTLADIDDDNIYPGLRVKTTYRPR
RGHIVWGLRDECATDSLNTLGNQSVVAYHRNNQTPINTDLLDAIEAADRR
VVETRRQGHGDDAVSFPQELLAVEPNTHQIKQFASDGEHQQARSKTRLSA
SRCSEKAQAFAERLDPVRLNGSTVEFSSEFFTGNNEQQLRLLYENGESVL
TERDGARGAHPDETESKGIVNPPESFEVAVVLPEQQADTCKAQWDTMADL
LNQAGAPPTRSETVQYDAFSSPESISLNVAGAIDPSEVDAAFVVLPPDQE
GFADLASPTETYDELKKALANMGIYSQMAYFDRERDAKIFYTRNVALGLL
AAAGGVAFTTEHAMPGDADMFIGIDVSRSYPEDGASGQINIAATATAVYK
DGTILGHSSTRPQLGEKLQSTDVRDIMKNAILGYQQVTGESPTHIVIHRD
GFMNEDLDPATEFLNEQGVEYDIVEIRKQPQTRLLAVSDVQYDTPVKSIA
AINQNEPRATVATFGAPEYLATRDGGGLPRPIQIERVAGETDIETLTRQV
YLLSQSHIQVHNSTARLPITTAYADQASTHATKGYLVQTGAFESNVGFLK
RPAATKKAGQAKKKKYPYDVPDYA*

>SEQ ID NO: 2 (2NLS-NgAgo-mNeonGreen: WT NgAgo amended with N- and C-terminal sequences for optimal translation, nuclear localization, and antibody detection, and fluorescent reporter fusion)

MASSPKKKRKVMTVIDLDSTTTADELTSGHTYDISVTLTGVYDNTDEQHP
RMSLAFEQDNGERRYITLWKNTTPKDVFTYDYATGSTYIFTNIDYEVKDG
YENLTATYQTTVENATAQEVGTTDEDETFAGGEPLDHHLDDALNETPDDA
ETESDSGHVMTSFASRDQLPEWTLHTYTLTATDGAKTDTEYARRTLAYTV
RQELYTDHDAAPVATDGLMLLTPEPLGETPLDLDCGVRVEADETRTLDYT
TAKDRLLARELVEEGLKRSLWDDYLVRGIDEVLSKEPVLTCDEFDLHERY
DLSVEVGHSGRAYLHINFRHRFVPKLTLADIDDDNIYPGLRVKTTYRPRR
GHIVWGLRDECATDSLNTLGNQSVVAYHRNNQTPINTDLLDAIEAADRRV
VETRRQGHGDDAVSFPQELLAVEPNTHQIKQFASDGFHQQARSKTRLSAS
RCSEKAQAFAERLDPVRLNGSTVEFSSEFFTGNNEQQLRLLYENGESVLT
FRDGARGAHPDETFSKGIVNPPESFEVAVVLPEQQADTCKAQWDTMADLL
NQAGAPPTRSETVQYDAFSSPESISLNVAGAIDPSEVDAAFVVLPPDQEG
FADLASPTETYDELKKALANMGIYSQMAYFDRFRDAKIFYTRNVALGLLA
AAGGVAFTTEHAMPGDADMFIGIDVSRSYPEDGASGQINIAATATAVYKD
GTILGHSSTRPQLGEKLQSTDVRDIMKNAILGYQQVTGESPTHIVIHRDG
FMNEDLDPATEFLNEQGVEYDIVEIRKQPQTRLLAVSDVQYDTPVKSIAA
INQNEPRATVATFGAPEYLATRDGGGLPRPIQIERVAGETDIETLTRQVY
LLSQSHIQVHNSTARLPITTAYADQASTHATKGYLVQTGAFESNVGFLKR
PAATKKAGQAKKKKYPYDVPDYAMVSKGEEDNMASLPATHELHIFGSING
VDFDMVGQGTGNPNDGYEELNLKSTKGDLQFSPWILVPHIGYGFHQYLPY
PDGMSPFQAAMVDGSGYQVHRTMQFEDGASLTVNYRYTYEGSHIKGEAQV
KGTGFPADGPVMTNSLTAADWCRSKKTYPNDKTIISTFKWSYTTGNGKRY
RSTARTTYTFAKPMAANYLKNQPMYVFRKTELKHSKTELNFKEWQKAFTD
VMGMDELYK*

>SEQ ID NO: 3 (strong constitutive expression cassette for 2NLS-NgAgo) Proprietary strong constitutive promoter configuration driving expression of this coding DNA sequence:

ATGGCgTCCTCCCCAAAGAAGAAGCGTAAGGTCATGACTGTTATCGACCT
TGATTCTACTACAACCGCTGACGAACTTACTTCCGGACACACCTACGACA
TTTCGGTTACTCTTACCGGCGTTTACGACAATACTGATGAGCAACACCCC
AGGATGTCCCTTGCATTCGAACAAGACAACGGCGAGAGAAGGTACATCAC
TCTGTGGAAAAACACTACACCTAAGGACGTGTTCACCTACGATTACGCAA
CCGGGAGTACATACATCTTTACAAACATCGACTACGAGGTAAAGGACGGG
TACGAAAACCTAACAGCTACTTACCAGACCACTGTCGAGAATGCTACAGC
CCAAGAGGTGGGCACCACCGACGAGGATGAAACATTCGCCGGAGGTGAAC
CTCTGGACCATCACCTTGATGATGCTTTAAACGAAACCCCTGACGATGCA
GAGACTGAGTCCGACTCCGGACACGTGATGACTTCCTTTGCATCTAGGGA
TCAGCTACCTGAGTGGACTCTTCACACCTACACCCTGACAGCTACTGACG
GAGCCAAAACCGATACTGAGTACGCCAGGCGTACCCTTGCTTACACAGTC
AGACAAGAACTATACACTGACCATGATGCCGCTCCAGTCGCTACCGATGG
ACTGATGCTTCTTACACCTGAACCACTGGGCGAAACACCACTTGACCTTG
ATTGCGGCGTGAGGGTGGAAGCCGACGAAACTCGCACACTGGACTACACC
ACCGCTAAAGATCGGTTACTCGCCAGAGAGCTTGTAGAAGAGGGACTTAA
ACGTAGTTTATGGGACGATTACCTTGTTAGAGGTATCGACGAGGTCCTCA
GTAAGGAACCTGTCCTTACCTGCGACGAGTTTGATCTTCATGAGAGGTAC
GACCTTTCTGTGGAAGTCGGACATTCGGGGAGGGCATACCTTCATATTAA
CTTCCGTCATCGTTTTGTACCTAAACTAACACTGGCTGACATCGACGATG
ACAACATTTACCCAGGACTTCGTGTCAAAACAACCTACCGGCCCCGTCGT
GGTCACATTGTCTGGGGACTTCGGGACGAGTGCGCAACAGACTCTCTTAA
TACCCTCGGAAACCAAAGTGTTGTGGCTTACCATAGGAACAACCAAACAC
CAATTAACACTGACCTTCTCGACGCTATCGAAGCCGCTGATCGCCGGGTT
GTGGAGACACGTAGACAAGGTCATGGGGACGACGCTGTGTCCTTCCCACA
AGAGCTTCTGGCTGTTGAACCCAACACCCATCAGATCAAGCAATTCGCTT
CCGATGGCTTCCATCAACAAGCCAGGTCTAAGACACGTCTTTCGGCTTCT
CGGTGCTCCGAGAAAGCCCAAGCATTTGCTGAACGTCTTGACCCTGTCCG
TCTTAACGGCTCTACTGTCGAGTTTAGTTCCGAGTTCTTCACCGGAAACA
ATGAACAGCAACTGAGACTTCTCTACGAAAATGGGGAATCGGTCCTTACA
TTTCGTGATGGAGCCAGGGGAGCCCATCCAGATGAGACATTCTCGAAAGG
CATTGTAAATCCACCCGAATCCTTTGAAGTCGCTGTCGTCCTTCCTGAAC
AACAGGCTGATACCTGCAAGGCTCAGTGGGACACCATGGCTGATCTACTC
AACCAAGCAGGCGCTCCTCCTACAAGGAGTGAAACAGTCCAGTACGATGC
CTTCTCCAGTCCCGAGAGTATTAGTCTTAACGTTGCTGGAGCCATTGACC
CATCCGAGGTGGATGCCGCTTTCGTGGTACTTCCACCAGACCAAGAAGGA
TTCGCTGACCTGGCTTCCCCAACAGAGACATACGACGAACTGAAAAAGGC
TCTTGCTAACATGGGAATCTACAGTCAAATGGCTTACTTCGACCGTTTTC
GCGACGCTAAAATCTTCTACACCCGTAATGTCGCCCTTGGCCTGCTTGCA
GCCGCTGGAGGTGTCGCATTTACAACAGAACATGCTATGCCTGGAGATGC
TGACATGTTTATCGGGATCGACGTTTCCAGGTCTTACCCTGAAGATGGAG
CCAGCGGACAAATCAACATCGCAGCTACTGCAACCGCTGTCTACAAGGAC
GGAACCATCCTTGGACACAGTTCCACTCGTCCACAATTAGGAGAAAAACT
TCAATCCACCGATGTCAGGGATATTATGAAGAACGCCATCCTCGGATACC
AACAAGTGACCGGAGAATCTCCTACCCACATTGTGATTCATCGTGACGGC
TTCATGAACGAGGACTTAGATCCTGCCACAGAGTTTCTAAACGAACAAGG
CGTCGAGTACGATATCGTTGAAATTCGCAAGCAACCTCAAACCAGGCTAT
TAGCCGTAAGTGATGTTCAATACGACACACCTGTCAAGTCCATTGCTGCT
ATCAACCAAAACGAACCACGCGCTACCGTGGCCACCTTTGGCGCCCCTGA
GTACCTTGCTACACGCGATGGTGGCGGCTTACCTAGACCTATTCAAATCG
AGCGCGTCGCTGGAGAAACAGATATCGAAACTCTTACAAGGCAAGTGTAC
CTTCTTTCTCAGAGTCACATCCAGGTCCATAACTCCACCGCTCGGCTCCC
TATCACAACTGCCTACGCTGACCAGGCTTCGACCCATGCTACAAAAGGAT
ACTTAGTCCAAACCGGAGCCTTTGAATCCAACGTGGGGTTCCTGAAGCGC
CCTGCTGCCACCAAAAAGGCTGGACAAGCCAAAAAAAAGAAGTACCCATA
CGATGTACCAGATTACGCTTAATCTAGAGGTACCTGATCATGAGTAATTA
GCTCGAATTTCCCCGATCGTTCAAACATTTGGCAATAAAGTTTCTTAAGA
TTGAATCCTGTTGCCGGTCTTGCGATGATTATCATATAATTTCTGTTGAA
TTACGTTAAGCATGTAATAATTAACATGTAATGCATGACGTTATTTATGA
GATGGGTTTTTATGATTAGAGTCCCGCAATTATACATTTAATACGCGATA
GAAAACAAAATATAGCGCGCAAACTAGGATAAATTATCGCGCGCGGTGTC
ATCTATGTTACTAGATCGCTCGACGCGGCCGCCATGGCCTCTAGTGGATC
ACCTAGGGTCGATCGACAAGCTCGAGTTTCTCCATAATAATGTGTGAGTA
GTTCCCAGATAAGGGAATTAGGGTTCCTATAGGGTTTCGCTCATGTGTTG
AGCATATAAGAAACCCTTAGTATGTATTTGTATTTGTAAAATACTTCTAT
CAATAAAATTTCTAATTCCTAAAACCAAAATCCAGTACTAAAATCCAGAT
CCCCCGAATTA

>SEQ ID NO: 4 (strong constitutive expression cassette for 2NLS-NgAgo-mNeonGreen) Proprietary strong constitutive promoter configuration driving expression of this coding DNA sequence:

ATGGCgTCCTCCCCAAAGAAGAAGCGTAAGGTCATGACTGTTATCGACCT
TGATTCTACTACAACCGCTGACGAACTTACTTCCGGACACACCTACGACA
TTTCGGTTACTCTTACCGGCGTTTACGACAATACTGATGAGCAACACCCC
AGGATGTCCCTTGCATTCGAACAAGACAACGGCGAGAGAAGGTACATCAC
TCTGTGGAAAAACACTACACCTAAGGACGTGTTCACCTACGATTACGCAA
CCGGGAGTACATACATCTTTACAAACATCGACTACGAGGTAAAGGACGGG
TACGAAAACCTAACAGCTACTTACCAGACCACTGTCGAGAATGCTACAGC
CCAAGAGGTGGGCACCACCGACGAGGATGAAACATTCGCCGGAGGTGAAC
CTCTGGACCATCACCTTGATGATGCTTTAAACGAAACCCCTGACGATGCA
GAGACTGAGTCCGACTCCGGACACGTGATGACTTCCTTTGCATCTAGGGA
TCAGCTACCTGAGTGGACTCTTCACACCTACACCCTGACAGCTACTGACG
GAGCCAAAACCGATACTGAGTACGCCAGGCGTACCCTTGCTTACACAGTC
AGACAAGAACTATACACTGACCATGATGCCGCTCCAGTCGCTACCGATGG
ACTGATGCTTCTTACACCTGAACCACTGGGCGAAACACCACTTGACCTTG
ATTGCGGCGTGAGGGTGGAAGCCGACGAAACTCGCACACTGGACTACACC
ACCGCTAAAGATCGGTTACTCGCCAGAGAGCTTGTAGAAGAGGGACTTAA
ACGTAGTTTATGGGACGATTACCTTGTTAGAGGTATCGACGAGGTCCTCA
GTAAGGAACCTGTCCTTACCTGCGACGAGTTTGATCTTCATGAGAGGTAC
GACCTTTCTGTGGAAGTCGGACATTCGGGGAGGGCATACCTTCATATTAA
CTTCCGTCATCGTTTTGTACCTAAACTAACACTGGCTGACATCGACGATG
ACAACATTTACCCAGGACTTCGTGTCAAAACAACCTACCGGCCCCGTCGT
GGTCACATTGTCTGGGGACTTCGGGACGAGTGCGCAACAGACTCTCTTAA
TACCCTCGGAAACCAAAGTGTTGTGGCTTACCATAGGAACAACCAAACAC
CAATTAACACTGACCTTCTCGACGCTATCGAAGCCGCTGATCGCCGGGTT
GTGGAGACACGTAGACAAGGTCATGGGGACGACGCTGTGTCCTTCCCACA
AGAGCTTCTGGCTGTTGAACCCAACACCCATCAGATCAAGCAATTCGCTT
CCGATGGCTTCCATCAACAAGCCAGGTCTAAGACACGTCTTTCGGCTTCT
CGGTGCTCCGAGAAAGCCCAAGCATTTGCTGAACGTCTTGACCCTGTCCG
TCTTAACGGCTCTACTGTCGAGTTTAGTTCCGAGTTCTTCACCGGAAACA
ATGAACAGCAACTGAGACTTCTCTACGAAAATGGGGAATCGGTCCTTACA
TTTCGTGATGGAGCCAGGGGAGCCCATCCAGATGAGACATTCTCGAAAGG
CATTGTAAATCCACCCGAATCCTTTGAAGTCGCTGTCGTCCTTCCTGAAC
AACAGGCTGATACCTGCAAGGCTCAGTGGGACACCATGGCTGATCTACTC
AACCAAGCAGGCGCTCCTCCTACAAGGAGTGAAACAGTCCAGTACGATGC
CTTCTCCAGTCCCGAGAGTATTAGTCTTAACGTTGCTGGAGCCATTGACC
CATCCGAGGTGGATGCCGCTTTCGTGGTACTTCCACCAGACCAAGAAGGA
TTCGCTGACCTGGCTTCCCCAACAGAGACATACGACGAACTGAAAAAGGC
TCTTGCTAACATGGGAATCTACAGTCAAATGGCTTACTTCGACCGTTTTC
GCGACGCTAAAATCTTCTACACCCGTAATGTCGCCCTTGGCCTGCTTGCA
GCCGCTGGAGGTGTCGCATTTACAACAGAACATGCTATGCCTGGAGATGC
TGACATGTTTATCGGGATCGACGTTTCCAGGTCTTACCCTGAAGATGGAG
CCAGCGGACAAATCAACATCGCAGCTACTGCAACCGCTGTCTACAAGGAC
GGAACCATCCTTGGACACAGTTCCACTCGTCCACAATTAGGAGAAAAACT
TCAATCCACCGATGTCAGGGATATTATGAAGAACGCCATCCTCGGATACC
AACAAGTGACCGGAGAATCTCCTACCCACATTGTGATTCATCGTGACGGC
TTCATGAACGAGGACTTAGATCCTGCCACAGAGTTTCTAAACGAACAAGG
CGTCGAGTACGATATCGTTGAAATTCGCAAGCAACCTCAAACCAGGCTAT
TAGCCGTAAGTGATGTTCAATACGACACACCTGTCAAGTCCATTGCTGCT
ATCAACCAAAACGAACCACGCGCTACCGTGGCCACCTTTGGCGCCCCTGA
GTACCTTGCTACACGCGATGGTGGCGGCTTACCTAGACCTATTCAAATCG
AGCGCGTCGCTGGAGAAACAGATATCGAAACTCTTACAAGGCAAGTGTAC
CTTCTTTCTCAGAGTCACATCCAGGTCCATAACTCCACCGCTCGGCTCCC
TATCACAACTGCCTACGCTGACCAGGCTTCGACCCATGCTACAAAAGGAT
ACTTAGTCCAAACCGGAGCCTTTGAATCCAACGTGGGGTTCCTGAAGCGC
CCTGCTGCCACCAAAAAGGCTGGACAAGCCAAAAAAAAGAAGTACCCATA
CGATGTACCAGATTACGCTATGGTGAGTAAAGGAGAAGAAGATAACATGG
CTTCGCTTCCAGCCACACATGAGCTTCACATCTTCGGTTCCATCAACGGC
GTTGACTTCGATATGGTCGGACAAGGCACTGGGAACCCTAATGACGGATA
CGAAGAGCTGAACCTCAAGAGCACCAAAGGTGATCTTCAGTTTTCTCCAT
GGATTCTGGTGCCACACATTGGCTACGGATTCCATCAATACCTTCCATAC
CCTGACGGAATGAGTCCATTCCAAGCAGCCATGGTTGATGGCTCCGGATA
CCAAGTCCACAGGACAATGCAGTTTGAGGACGGTGCTTCGCTCACCGTCA
ACTACCGTTACACTTACGAAGGGAGCCACATCAAAGGAGAAGCCCAAGTG
AAGGGGACAGGCTTTCCTGCTGATGGACCTGTCATGACCAACTCCTTAAC
TGCCGCTGATTGGTGCCGGTCCAAGAAAACCTACCCTAACGACAAGACCA
TCATTAGTACCTTCAAATGGTCTTACACCACAGGCAATGGCAAGAGATAT
CGCTCTACAGCCAGGACTACCTACACATTCGCTAAACCAATGGCCGCTAA
CTACCTTAAGAACCAACCCATGTACGTGTTCCGTAAGACTGAGTTGAAAC
ATTCCAAGACCGAACTTAACTTCAAGGAGTGGCAGAAGGCATTTACCGAC
GTAATGGGCATGGATGAACTATACAAATAATCTAGAGGTACCTGATCATG
AGTAATTAGCTCGAATTTCCCCGATCGTTCAAACATTTGGCAATAAAGTT
TCTTAAGATTGAATCCTGTTGCCGGTCTTGCGATGATTATCATATAATTT
CTGTTGAATTACGTTAAGCATGTAATAATTAACATGTAATGCATGACGTT
ATTTATGAGATGGGTTTTTATGATTAGAGTCCCGCAATTATACATTTAAT
ACGCGATAGAAAACAAAATATAGCGCGCAAACTAGGATAAATTATCGCGC
GCGGTGTCATCTATGTTACTAGATCGCTCGACGCGGCCGCCATGGCCTCT
AGTGGATCACCTAGGGTCGATCGACAAGCTCGAGTTTCTCCATAATAATG
TGTGAGTAGTTCCCAGATAAGGGAATTAGGGTTCCTATAGGGTTTCGCTC
ATGTGTTGAGCATATAAGAAACCCTTAGTATGTATTTGTATTTGTAAAAT
ACTTCTATCAATAAAATTTCTAATTCCTAAAACCAAAATCCAGTACTAAA
ATCCAGATCCCCCGAATTA

### >SEQ ID NO: 5

### GenBank: AFZ73749.1

>gi|429136738|gb|AFZ73749.1| uncharacterized protein containing piwi/argonaute domain [Natronobacterium gregoryi SP2]

MTVIDLDSTTTADELTSGHTYDISVTLTGVYDNTDEQHPRMSLAFEQDNG
ERRYITLWKNTTPKDVFTYDYATGSTYIFTNIDYEVKDGYENLTATYQTT
VENATAQEVGTTDEDETFAGGEPLDHHLDDALNETPDDAETESDSGHVMT
SFASRDQLPEWTLHTYTLTATDGAKTDTEYARRTLAYTVRQELYTDHDAA
PVATDGLMLLTPEPLGETPLDLDCGVRVEADETRTLDYTTAKDRLLAREL
VEEGLKRSLWDDYLVRGIDEVLSKEPVLTCDEFDLHERYDLSVEVGHSGR
AYLHINFRHRFVPKLTLADIDDDNIYPGLRVKTTYRPRRGHIVWGLRDEC
ATDSLNTLGNQSVVAYHRNNQTPINTDLLDAIEAADRRVVETRRQGHGDD
AVSFPQELLAVEPNTHQIKQFASDGFHQQARSKTRLSASRCSEKAQAFAE
RLDPVRLNGSTVEFSSEFFTGNNEQQLRLLYENGESVLTFRDGARGAHPD
ETFSKGIVNPPESFEVAVVLPEQQADTCKAQWDTMADLLNQAGAPPTRSE
TVQYDAFSSPESISLNVAGAIDPSEVDAAFVVLPPDQEGFADLASPTETY
DELKKALANMGIYSQMAYFDRFRDAKIFYTRNVALGLLAAAGGVAFTTEH
AMPGDADMFIGIDVSRSYPEDGASGQINIAATATAVYKDGTILGHSSTRP
QLGEKLQSTDVRDIMKNAILGYQQVTGESPTHIVIHRDGFMNEDLDPATE
FLNEQGVEYDIVEIRKQPQTRLLAVSDVQYDTPVKSIAAINQNEPRATVA
TFGAPEYLATRDGGGLPRPIQIERVAGETDIETLTRQVYLLSQSHIQVHN
STARLPITTAYADQASTHATKGYLVQTGAFESNVGFL

### >SEQ ID NO: 6

tr|L0AJX6|L0AJX6_NATGS Stem cell self-renewal protein Piwi domain protein OS═Natronobacterium gregoryi (strain ATCC 43098/CCM 3738/NCIMB 2189/SP2) GN=Natgr_2597 PE=4 SV=1

MTVIDLDSTTTADELTSGHTYDISVTLTGVYDNTDEQHPRMSLAFEQDNG
ERRYITLWKNTTPKDVFTYDYATGSTYIFTNIDYEVKDGYENLTATYQTT
VENATAQEVGTTDEDETFAGGEPLDHHLDDALNETPDDAETESDSGHVMT
SFASRDQLPEWTLHTYTLTATDGAKTDTEYARRTLAYTVRQELYTDHDAA
PVATDGLMLLTPEPLGETPLDLDCGVRVEADETRTLDYTTAKDRLLAREL
VEEGLKRSLWDDYLVRGIDEVLSKEPVLTCDEFDLHERYDLSVEVGHSGR
AYLHINFRHRFVPKLTLADIDDDNIYPGLRVKTTYRPRRGHIVWGLRDEC
ATDSLNTLGNQSVVAYHRNNQTPINTDLLDAIEAADRRVVETRRQGHGDD
AVSFPQELLAVEPNTHQIKQFASDGFHQQARSKTRLSASRCSEKAQAFAE
RLDPVRLNGSTVEFSSEFFTGNNEQQLRLLYENGESVLTFRDGARGAHPD
ETFSKGIVNPPESFEVAVVLPEQQADTCKAQWDTMADLLNQAGAPPTRSE
TVQYDAFSSPESISLNVAGAIDPSEVDAAFVVLPPDQEGFADLASPTETY
DELKKALANMGIYSQMAYFDRFRDAKIFYTRNVALGLLAAAGGVAFTTEH
AMPGDADMFIGIDVSRSYPEDGASGQINIAATATAVYKDGTILGHSSTRP
QLGEKLQSTDVRDIMKNAILGYQQVTGESPTHIVIHRDGFMNEDLDPATE
FLNEQGVEYDIVEIRKQPQTRLLAVSDVQYDTPVKSIAAINQNEPRATVA
TFGAPEYLATRDGGGLPRPIQIERVAGETDIETLTRQVYLLSQSHIQVHN
STARLPITTAYADQASTHATKGYLVQTGAFESNVGFL

### >SEQ ID NO: 7

>ENA|AFZ73749|AFZ73749.1 Natronobacterium gregoryi SP2 uncharacterized protein containing piwi/argonaute domain

ATGACAGTGATTGACCTCGATTCGACCACCACCGCAGACGAACTGACATC
GGGACACACGTACGACATCTCAGTCACGCTCACCGGTGTCTACGATAACA
CCGACGAGCAGCATCCTCGCATGTCTCTCGCATTCGAGCAGGACAACGGC
GAGCGGCGTTACATTACCCTGTGGAAGAACACGACACCCAAGGATGTCTT
TACATACGACTACGCCACGGGCTCGACGTACATCTTCACTAACATCGACT
ACGAAGTGAAGGACGGCTACGAGAATCTGACTGCAACATACCAGACGACC
GTCGAGAACGCTACCGCTCAGGAAGTCGGGACGACTGACGAGGACGAAAC
GTTCGCGGGCGGCGAGCCGCTCGACCATCACTTGGACGACGCGCTCAATG
AGACGCCAGACGACGCGGAGACAGAGAGCGACTCAGGCCATGTGATGACC
TCGTTCGCCTCCCGCGACCAACTCCCTGAGTGGACGCTGCATACGTATAC
GCTAACAGCCACAGACGGCGCAAAGACGGACACGGAGTACGCGCGACGAA
CCCTCGCATACACGGTACGGCAGGAACTCTATACCGACCATGATGCGGCT
CCGGTTGCAACTGACGGGCTAATGCTTCTCACGCCAGAGCCGCTCGGCGA
GACCCCGCTTGACCTCGATTGCGGTGTCCGGGTCGAGGCGGACGAGACTC
GGACACTCGATTACACCACGGCCAAAGACCGGTTACTCGCCCGCGAACTC
GTCGAAGAGGGGCTCAAACGCTCCCTCTGGGATGACTACCTCGTTCGCGG
CATCGATGAAGTCCTCTCAAAGGAGCCTGTGCTGACTTGCGATGAGTTCG
ACCTACATGAGCGGTATGACCTCTCTGTCGAAGTCGGTCACAGTGGGCGG
GCGTACCTTCACATCAACTTCCGCCACCGGTTCGTACCGAAGCTGACGCT
CGCAGACATCGATGATGACAACATCTATCCTGGGCTCCGGGTGAAGACGA
CGTATCGCCCCCGGCGAGGACATATCGTCTGGGGTCTGCGGGACGAGTGC
GCCACCGACTCGCTCAACACGCTGGGAAACCAGTCCGTCGTTGCATACCA
CCGCAACAATCAGACACCTATTAACACTGACCTCCTCGACGCTATCGAGG
CCGCTGACCGGCGAGTCGTCGAAACCCGACGTCAAGGGCACGGCGATGAT
GCTGTCTCATTCCCCCAAGAACTGCTTGCGGTCGAACCGAATACGCACCA
AATTAAGCAGTTCGCCTCCGACGGATTCCACCAACAGGCCCGCTCAAAGA
CGCGTCTCTCGGCCTCCCGCTGCAGCGAGAAAGCGCAAGCGTTCGCCGAG
CGGCTTGACCCGGTGCGTCTCAATGGGTCCACGGTAGAGTTCTCCTCGGA
GTTTTTCACCGGGAACAACGAGCAGCAACTGCGCCTCCTCTACGAGAACG
GTGAGTCGGTTCTGACGTTCCGCGACGGGGCGCGTGGTGCGCACCCCGAC
GAGACATTCTCGAAAGGTATCGTCAATCCACCAGAGTCGTTCGAGGTGGC
CGTAGTACTGCCCGAGCAGCAGGCAGATACCTGCAAAGCGCAGTGGGACA
CGATGGCTGACCTCCTCAACCAAGCTGGCGCGCCACCGACACGGAGCGAG
ACCGTCCAATATGATGCGTTCTCCTCGCCAGAGAGCATCAGCCTCAATGT
GGCTGGAGCCATCGACCCTAGCGAGGTAGACGCGGCATTCGTCGTACTGC
CGCCGGACCAAGAAGGATTCGCAGACCTCGCCAGTCCGACAGAGACGTAC
GACGAGCTGAAGAAGGCGCTTGCCAACATGGGCATTTACAGCCAGATGGC
GTACTTCGACCGGTTCCGCGACGCGAAAATATTCTATACTCGTAACGTGG
CACTCGGGCTGCTGGCAGCCGCTGGCGGCGTCGCATTCACAACCGAACAT
GCGATGCCTGGGGACGCAGATATGTTCATTGGGATTGATGTCTCTCGGAG
CTACCCCGAGGACGGTGCCAGCGGCCAGATAAACATTGCCGCGACGGCGA
CCGCCGTCTACAAGGATGGAACTATCCTCGGCCACTCGTCCACCCGACCG
CAGCTCGGGGAGAAACTACAGTCGACGGATGTTCGTGACATTATGAAGAA
TGCCATCCTCGGCTACCAGCAGGTGACCGGTGAGTCGCCGACCCATATCG
TCATCCACCGTGATGGCTTCATGAACGAAGACCTCGACCCCGCCACGGAA
TTCCTCAACGAACAAGGCGTCGAGTACGACATCGTCGAAATCCGCAAGCA
GCCCCAGACACGCCTGCTGGCAGTCTCCGATGTGCAGTACGATACGCCTG
TGAAGAGCATCGCCGCTATCAACCAGAACGAGCCACGGGCAACGGTCGCC
ACCTTCGGCGCACCCGAATACTTAGCGACACGCGATGGAGGCGGCCTTCC
CCGCCCAATCCAAATTGAACGAGTCGCCGGCGAAACCGACATCGAGACGC
TCACTCGCCAAGTCTATCTGCTCTCCCAGTCGCATATCCAGGTCCATAAC
TCGACTGCGCGCCTACCCATCACCACCGCATACGCCGACCAGGCAAGTAC
TCACGCGACCAAGGGTTACCTCGTCCAGACCGGAGCGTTCGAGTCTAATG
TCGGATTCCTCTAA

The present invention is not to be limited in scope by the specific embodiments described herein. Indeed, various modifications of the invention in addition to those described herein will become apparent to those skilled in the art from the foregoing description. Such modifications are intended to fall within the scope of the appended claims.

All patents, applications, publications, test methods, literature, and other materials cited herein are hereby incorporated by reference in their entirety as if physically present in this specification.

1. A method of modifying chromosomal or extrachromosomal genetic material in a eukaryotic cell, comprising:
a. introducing into the cell a nucleic acid-targeting nucleic acid that is directed against a target sequence within the cell chromosomal or extrachromosomal genetic material; and
b. introducing into the cell an Argonaute endonuclease that produces a single- or double-strand break at or near the target site of the nucleic acid-targeting nucleic acid.

2. The method of claim 1, wherein the nucleic acid-targeting nucleic acid is a 5′-phosphorylated, single-stranded DNA.

3. The method of claim 1, wherein the nucleic acid-targeting nucleic acid has the length selected from the group consisting of 20, 21, 22, 23, 24, 25, 26, 27, 28, 29, and 30 nucleotides.

4. The method of claim 1, wherein the nucleic acid-targeting nucleic acid is comprised of conventional deoxyribonucleic acid nucleotides and standard phosphate backbone linkages.

5. (canceled)

6. (canceled)

7. The method of claim 1, wherein the Argonaute endonuclease is the Natronobacterium gregoryi Argonaute endonuclease (NgAgo) or a mutant or a derivative thereof.

8. The method of claim 7, wherein the NgAgo is modified to express nickase activity or to have DNA targeting activity without any nickase or nuclease activity.

9. The method of claim 7, wherein at least one additional protein domain with enzymatic activity is fused to the N- or C-terminus, or both, of the NgAgo endonuclease.

10. (canceled)

11. (canceled)

12. (canceled)

13. (canceled)

14. (canceled)

15. (canceled)

16. (canceled)

17. (canceled)

18. (canceled)

19. The method of claim 1, wherein the eukaryotic cell is a plant cell.

20. The method of claim 19, wherein the Argonaute endonuclease and/or the nucleic acid-targeting guide nucleic acid is delivered to the plant cell by a method selected from the group consisting of bacteria-mediated DNA transfer, microparticle bombardment into plant cells, polyethylene glycol (PEG) mediated transformation of plant cells, electroporation of plant cells, pollen-tube mediated introduction into zygotes, and delivery mediated by one or more cell-penetrating peptides (CPPs).

21. The method of claim 19, wherein the Argonaute endonuclease and/or the nucleic acid-targeting guide nucleic acid is delivered to the plant cell by Agrobacterium-mediated transformation.

22. The method of claim 19, wherein the plant cell is derived from a species selected from the group consisting of Hordeum vulgare, Hordeum bulbusom, Sorghum bicolor, Saccharum officinarium, Zea mays, Setaria italica, Oryza minuta, Oriza sativa, Oryza australiensis, Oryza alta, Triticum aestivum, Triticum durum, Secale cereale, Triticale, Malus domestica, Brachypodium distachyon, Hordeum marinum, Aegilops tauschii, Daucus glochidiatus, Beta vulgaris, Daucus pusillus, Daucus muricatus, Daucus carota, Eucalyptus grandis, Nicotiana sylvestris, Nicotiana tomentosiformis, Nicotiana tabacum, Nicotiana benthamiana, Solanum lycopersicum, Solanum tuberosum, Coffea canephora, Vitis vinifera, Erythrante guttata, Genlisea aurea, Cucumis sativus, Morus notabilis, Arabidopsis arenosa, Arabidopsis lyrata, Arabidopsis thaliana, Crucihimalaya himalaica, Crucihimalaya wallichii, Cardamine flexuosa, Lepidium virginicum, Capsella bursa pastoris, Olmarabidopsis pumila, Arabis hirsute, Brassica napus, Brassica oleracea, Brassica rapa, Raphanus sativus, Brassica juncacea, Brassica nigra, Eruca vesicaria subsp. sativa, Citrus sinensis, Jatropha curcas, Populus trichocarpa, Medicago truncatula, Cicer yamashitae, Cicer bijugum, Cicer arietinum, Cicer reticulatum, Cicer judaicum, Cajanus cajanifolius, Cajanus scarabaeoides, Phaseolus vulgaris, Glycine max, Gossypium sp., Astragalus sinicus, Lotus japonicas, Torenia fournieri, Allium cepa, Allium fistulosum, Allium sativum, Helianthus annuus, Helianthus tuberosus and Allium tuberosum, and any variety or subspecies belonging to one of the aforementioned plants.

23. The method of claim 19, wherein the target sequence is selected from the group consisting of an acetolactate synthase (ALS) gene, an acetohydroxyacid synthase (AHAS) gene, an enolpyruvylshikimate phosphate synthase gene (EPSPS) gene, male fertility genes, male sterility genes, female fertility genes, female sterility genes, male restorer genes, female restorer genes, genes associated with the traits of sterility, genes associated with the traits of fertility, genes associated with herbicide resistance, genes associated with herbicide tolerance, genes associated with fungal resistance, genes associated with viral resistance, genes associated with insect resistance, genes associated with drought tolerance, genes associated with chilling tolerance, genes associated with cold tolerance, genes associated with nitrogen use efficiency, genes associated with phosphorus use efficiency, genes associated with water use efficiency and genes associated with crop or biomass yield, and any mutants of such genes.

24. (canceled)

25. The method of claim 1, wherein the Argonaute endonuclease is modified so as to be active at a different temperature than its optimal temperature prior to modification.

26. The method of claim 25, wherein the modified Argonaute endonuclease is active at temperatures suitable for growth and culture of plants and plant cells.

27. The method of claim 25, wherein the modified Argonaute endonuclease is active at a temperature from about 20° C. to about 35° C.

28. (canceled)

29. (canceled)

30. (canceled)

31. (canceled)

32. (canceled)

33. (canceled)

34. (canceled)

35. (canceled)

36. A method for treating a disease or condition and/or preventing insect infection/infestation in a plant comprising modifying chromosomal or extrachromosomal genetic material of said plant by use of the method of claim 1.

37. A method for affecting at least one trait in a plant selected from the group consisting of sterility, fertility, herbicide resistance, herbicide tolerance, fungal resistance, viral resistance, insect resistance, drought tolerance, chilling tolerance, or cold tolerance, nitrogen use efficiency, phosphorus use efficiency, water use efficiency and crop or biomass yield, said method comprising modifying chromosomal or extrachromosomal genetic material of said plant by use of the method of claim 1.

38. The method of claim 36, wherein the disease or condition is selected from the group consisting of Anthracnose Stalk Rot, Aspergillus Ear Rot, Common Corn Ear Rots, Corn Ear Rots (Uncommon), Common Rust of Corn, Diplodia Ear Rot, Diplodia Leaf Streak, Diplodia Stalk Rot, Downy Mildew, Eyespot, Fusarium Ear Rot, Fusarium Stalk Rot, Gibberella Ear Rot, Gibberella Stalk Rot, Goss's Wilt and Leaf Blight, Gray Leaf Spot, Head Smut, Northern Corn Leaf Blight, Physoderma Brown Spot, Pythium, Southern Leaf Blight, Southern Rust, and Stewart's Bacterial Wilt and Blight, and combinations thereof.

39. The method of claim 36, wherein the disease or condition is directly or indirectly caused by, and/or the insect infection/infestation results from, at least one insect selected from the group consisting of Armyworm, Asiatic Garden Beetle, Black Cutworm, Brown Marmorated Stink Bug, Brown Stink Bug, Common Stalk Borer, Corn Billbugs, Corn Earworm, Corn Leaf Aphid, Corn Rootworm, Corn Rootworm Silk Feeding, European Corn Borer, Fall Armyworm, Grape Colaspis, Hop Vine Borer, Japanese Beetle, Scouting for Fall Armyworm, Seedcorn Beetle, Seedcorn Maggot, Southern Corn Leaf Beetle, Southwestern Corn Borer, Spider Mite, Sugarcane Beetle, Western Bean Cutworm, White Grub, and Wireworms, and combinations thereof.
